SEQUENCE LISTING (106 sequences)

SEQ ID NO: 1 - protein, 426 aa, Homo sapiens
MFRTKRSALVRRLWRSRAPGGEDEEEGAGGGGGGGELRGEGATDSRAHGAGGGGPGRAGCCLGKAVRGAKGHHHPHPPAAGAGAAGGAEADLKALTHSVLKKLKERQLELLLQAVESRGGTRTACLLLPGRLDCRLGPGAPAGAQPAQPPSSYSLPLLLCKVFRWPDLRHSSEVKRLCCCESYGKINPELVCCNPHHLSRLCELESPPPPYSRYPMDFLKPTADCPDAVPSSAETGGTNYLAPGGLSDSQLLLEPGDRSHWCVVAYWEEKTRVGRLYCVQEPSLDIFYDLPQGNGFCLGQLNSDNKSQLVQKVRSKIGCGIQLTREVDGVWVYNRSSYPIFIKSATLDNPDSRTLLVHKVFPGFSIKAFDYEKAYSLQRPNDHEFMQQPWTGFTVQISFVKGWGQCYTRQFISSCPCWLEVIFNSR

SEQ ID NO: 2 - DNA, 1281 bp, Homo sapiens
atgttcaggaccaaacgatctgcgctcgtccggcgtctctggaggagccgtgcgcccggcggcgaggacgaggaggagggcgcagggggaggtggaggaggaggcgagctgcggggagaaggggcgacggacagccgagcgcatggggccggtggcggcggcccgggcagggctggatgctgcctgggcaaggcggtgcgaggtgccaaaggtcaccaccatccccacccgccagccgcgggcgccggcgcggccgggggcgccgaggcggatctgaaggcgctcacgcactcggtgctcaagaaactgaaggagcggcagctggagctgctgctccaggccgtggagtcccgcggcgggacgcgcaccgcgtgcctcctgctgcccggccgcctggactgcaggctgggcccgggggcgcccgccggcgcgcagcctgcgcagccgccctcgtcctactcgctccccctcctgctgtgcaaagtgttcaggtggccggatctcaggcattcctcggaagtcaagaggctgtgttgctgtgaatcttacgggaagatcaaccccgagctggtgtgctgcaacccccatcaccttagccgactctgcgaactagagtctcccccccctccttactccagatacccgatggattttctcaaaccaactgcagactgtccagatgctgtgccttcctccgctgaaacagggggaacgaattatctggcccctggggggctttcagattcccaacttcttctggagcctggggatcggtcacactggtgcgtggtggcatactgggaggagaagacgagagtggggaggctctactgtgtccaggagccctctctggatatcttctatgatctacctcaggggaatggcttttgcctcggacagctcaattcggacaacaagagtcagctggtgcagaaggtgcggagcaaaatcggctgcggcatccagctgacgcgggaggtggatggtgtgtgggtgtacaaccgcagcagttaccccatcttcatcaagtccgccacactggacaacccggactccaggacgctgttggtacacaaggtgttccccggtttctccatcaaggctttcgactacgagaaggcgtacagcctgcagcggcccaatgaccacgagtttatgcagcagccgtggacgggctttaccgtgcagatcagctttgtgaagggctggggtcagtgctacacccgccagttcatcagcagctgcccgtgctggctagaggtcatcttcaacagccggtag

SEQ ID NO: 3 - protein, 14 aa, Artificial Sequence
  Synthetic
GKPIPNPLLGLDST

SEQ ID NO: 4 - protein, 8 aa, Artificial Sequence
  Synthetic
KYKDDDDK

SEQ ID NO: 5 - protein, 9 aa, Artificial Sequence
  Synthetic
YPYDVPDYA

SEQ ID NO: 6 - protein, 10 aa, Human Immunodeficiency Virus
GRKKRRQRRR

SEQ ID NO: 7 - protein, 11 aa, Human Immunodeficiency Virus
YGRKKRRQRRR

SEQ ID NO: 8 - protein, 7 aa, Human Immunodeficiency Virus
GRKKRRQ

SEQ ID NO: 9 - protein, 23 aa, Artificial Sequence
  Synthetic
GALFLGWLGAAGSTMGAKKKRKV

SEQ ID NO: 10 - protein, 16 aa, Artificial Sequence
  Synthetic
RQIKIWFQNRRMKWKK

SEQ ID NO: 11 - protein, 7 aa, Artificial Sequence
  Synthetic
RRMKWKK

SEQ ID NO: 12 - protein, 16 aa, Artificial Sequence
  Synthetic
RRWRRWWRRWWRRWRR

SEQ ID NO: 13 - protein, 18 aa, Artificial Sequence
  Synthetic
RGGRLSYSRRRFSTSTGR

SEQ ID NO: 14 - protein, 9 aa, Artificial Sequence
  Synthetic
RKKRRQRRR

SEQ ID NO: 15 - protein, 11 aa, Artificial Sequence
  Synthetic
YARAAARQARA

SEQ ID NO: 16 - protein, 8 aa, Artificial Sequence
  Synthetic
RRRRRRRR

SEQ ID NO: 17 - protein, 8 aa, Artificial Sequence
  Synthetic
KKKKKKKK

SEQ ID NO: 18 - protein, 27 aa, Artificial Sequence
  Synthetic
  misc_feature (25)..(25): Xaa can be any naturally occurring amino acid
GWTLNSAGYLLGKINLKALAALAKXIL

SEQ ID NO: 19 - protein, 18 aa, Artificial Sequence
  Synthetic
LLILLRRRIRKQANAHSK

SEQ ID NO: 20 - protein, 16 aa, Artificial Sequence
  Synthetic
SRRHHCRSKAKRSRHH

SEQ ID NO: 21 - protein, 11 aa, Artificial Sequence
  Synthetic
NRARRNRRRVR

SEQ ID NO: 22 - protein, 15 aa, Artificial Sequence
  Synthetic
RQLRIAGRRLRGRSR

SEQ ID NO: 23 - protein, 13 aa, Artificial Sequence
  Synthetic
KLIKGRTPIKFGK

SEQ ID NO: 24 - protein, 10 aa, Artificial Sequence
  Synthetic
RRIPNRRPRR

SEQ ID NO: 25 - protein, 18 aa, Artificial Sequence
  Synthetic
KLALKLALKALKAALKLA

SEQ ID NO: 26 - protein, 14 aa, Artificial Sequence
  Synthetic
KLAKLAKKLAKLAK

SEQ ID NO: 27 - protein, 27 aa, Artificial Sequence
  Synthetic
GALFLGFLGAAGSTNGAWSQPKKKRKV

SEQ ID NO: 28 - protein, 21 aa, Artificial Sequence
  Synthetic
KETWWETWWTEWSQPKKKRKV

SEQ ID NO: 29 - protein, 20 aa, Artificial Sequence
  Synthetic
LKKLLKKLLKKLLKKLLKKL

SEQ ID NO: 30 - protein, 34 aa, Artificial Sequence
  Synthetic
QAATATRGRSAASRPTERPRAPARSASRPRRPVE

SEQ ID NO: 31 - protein, 23 aa, Artificial Sequence
  Synthetic
MGLGLHLLVLAAALQGAKSKRKV

SEQ ID NO: 32 - protein, 26 aa, Artificial Sequence
  Synthetic
AAVALLPAVLLALLAPAAANYKKPKL

SEQ ID NO: 33 - protein, 28 aa, Artificial Sequence
  Synthetic
MANLGYWLLALFVTMWTDVGLCKKRPKP

SEQ ID NO: 34 - protein, 24 aa, Artificial Sequence
  Synthetic
LGTYTQDFNKFHTFPQTAIGVGAP

SEQ ID NO: 35 - protein, 26 aa, Artificial Sequence
  Synthetic
  misc_feature (24)..(24): Xaa can be any naturally occurring amino acid
DPKGDPKGVTVTVTVTVTGKGDPXPD

SEQ ID NO: 36 - protein, 14 aa, Artificial Sequence
  Synthetic
PPPPPPPPPPPPPP

SEQ ID NO: 37 - protein, 18 aa, Artificial Sequence
  Synthetic
VRLPPPVRLPPPVRLPPP

SEQ ID NO: 38 - protein, 10 aa, Artificial Sequence
  Synthetic
PRPLPPPRPG

SEQ ID NO: 39 - protein, 30 aa, Artificial Sequence
  Synthetic
SVRRRPRPPYLPRPRPPPFFPPRLPPRIPP

SEQ ID NO: 40 - protein, 21 aa, Artificial Sequence
  Synthetic
TRSSRAGLQFPVGRVHRLLRK

SEQ ID NO: 41 - protein, 23 aa, Artificial Sequence
  Synthetic
GIGKFLHSAKKFGKAFVGEIMNS

SEQ ID NO: 42 - protein, 37 aa, Artificial Sequence
  Synthetic
KWKLFKKIEKVGQNIRDGIIKAGPAVAVVGQATQIAK

SEQ ID NO: 43 - protein, 28 aa, Artificial Sequence
  Synthetic
ALWMTLLKKVLKAAAKAALNAVLVGANA

SEQ ID NO: 44 - protein, 26 aa, Artificial Sequence
  Synthetic
GIGAVLKVLTTGLPALISWIKRKRQQ

SEQ ID NO: 45 - protein, 14 aa, Artificial Sequence
  Synthetic
INLKALAALAKKIL

SEQ ID NO: 46 - protein, 33 aa, Artificial Sequence
  Synthetic
GFFALIPKIISSPLPKTLLSAVGSALGGSGGQE

SEQ ID NO: 47 - protein, 15 aa, Artificial Sequence
  Synthetic
LAKWALKQGFAKLKS

SEQ ID NO: 48 - protein, 27 aa, Artificial Sequence
  Synthetic
  misc_feature (23)..(23): Xaa can be any naturally occurring amino acid
SMAQDIISTIGDLVKWIIQTVNXFTKK

SEQ ID NO: 49 - protein, 41 aa, Artificial Sequence
  Synthetic
LLGDFFRKSKEKIGKEFKRIVQRIKQRIKDFLANLVPRTES

SEQ ID NO: 50 - protein, 18 aa, Artificial Sequence
  Synthetic
PAWRKAFRWAWRMLKKAA

SEQ ID NO: 51 - protein, 18 aa, Artificial Sequence
  Synthetic
KLKLKLKLKLKLKLKLKL

SEQ ID NO: 52 - DNA, 30 bp, Human Immunodeficiency Virus
ggccgtaaaaaacgccgtcaacgccgccgt

SEQ ID NO: 53 - DNA, 33 bp, Human Immunodeficiency Virus
tatggccgtaaaaaacgccgtcaacgccgccgt

SEQ ID NO: 54 - DNA, 21 bp, Human Immunodeficiency Virus
ggccgtaaaaaacgccgtcaa

SEQ ID NO: 55 - protein, 7 aa, Human Immunodeficiency Virus
GRKKRRQ

SEQ ID NO: 56 - protein, 6 aa, Artificial Sequence
  Synthetic
HHHHHH

SEQ ID NO: 57 - DNA, 744 bp, Artificial Sequence
  Synthetic
ggatccggccgtaaaaaacgccgtcaacgccgccgtgaactggagagcccgccgccgccgtattctcgttacccgatggatttcctgaaaccgactgcagattgcccggacgcagtcccgtcatcggctgagaccggcggcaccaactatctggcaccgggcggtctgagtgattcccagctgctgctggaaccgggcgaccgttcacattggtgtgtggttgcctattgggaagagaaaacgcgtgtcggtcgcctgtactgcgtacaggaaccgtcgctggatatcttttatgacctgccgcagggcaatggtttctgtctgggccaactgaactcagataataaatcgcagctggtgcaaaaagttcgctcaaaaattggctgcggtatccagctgacccgtgaagttgacggtgtctgggtatataaccgcagctcttacccgatttttatcaaaagtgccaccctggataatccggactcccgtacgctgctggtccacaaagtatttccgggcttctcaatcaaagcgttcgattacgagaaagcctactcgctgcagcgcccgaacgaccatgaattcatgcagcaaccgtggacgggttttactgtgcagatctctttcgttaaaggctggggtcaatgctacacccgtcagtttatctcgtcctgtccgtgctggctggaagtgattttcaatagccgctacccatacgacgtcccagactacgcttaggtcgac

SEQ ID NO: 58 - protein, 245 aa, Artificial Sequence
  Synthetic
GSGRKKRRQRRRELESPPPPYSRYPMDFLKPTADCPDAVPSSAETGGTNYLAPGGLSDSQLLLEPGDRSHWCVVAYWEEKTRVGRLYCVQEPSLDIFYDLPQGNGFCLGQLNSDNKSQLVQKVRSKIGCGIQLTREVDGVWVYNRSSYPIFIKSATLDNPDSRTLLVHKVFPGFSIKAFDYEKAYSLQRPNDHEFMQQPWTGFTVQISFVKGWGQCYTRQFISSCPCWLEVIFNSRYPYDVPDYA

SEQ ID NO: 59 - DNA, 744 bp, Artificial Sequence
  Synthetic
ggatccggccgtaaaaaacgccgtcaacgccgccgtgaactggagagcccgccgccgccgtattctcgttacccgctggatttcctgaaaccgactgcagattgcccggacgcagtcccgtcatcggctgagaccggcggcaccaactatctggcaccgggcggtctgagtgattcccagctgctgctggaaccgggcgaccgttcacattggtgtgtggttgcctattgggaagagaaaacgcgtgtcggtcgcctgtactgcgtacaggaaccgtcgctggatatcttttatgacctgccgcagggcaatggtttctgtctgggccaactgaactcagataataaatcgcagctggtgcaaaaagttcgctcaaaaattggctgcggtatccagctgacccgtgaagttgacggtgtctgggtatataaccgcagctcttacccgatttttatcaaaagtgccaccctggataatccggactcccgtacgctgctggtccacaaagtatttccgggcttctcaatcaaagcgttcgattacgagaaagcctactcgctgcagcgcccgaacgaccatgaattcatgcagcaaccgtggacgggttttactgtgcagatctctttcgttaaaggctggggtcaatgctacacccgtcagtttatctcgtcctgtccgtgctggctggaagtgattttcaatagccgctacccatacgacgtcccagactacgcttaggtcgac

SEQ ID NO: 60 - protein, 245 aa, Artificial Sequence
  Synthetic
GSGRKKRRQRRRELESPPPPYSRYPLDFLKPTADCPDAVPSSAETGGTNYLAPGGLSDSQLLLEPGDRSHWCVVAYWEEKTRVGRLYCVQEPSLDIFYDLPQGNGFCLGQLNSDNKSQLVQKVRSKIGCGIQLTREVDGVWVYNRSSYPIFIKSATLDNPDSRTLLVHKVFPGFSIKAFDYEKAYSLQRPNDHEFMQQPWTGFTVQISFVKGWGQCYTRQFISSCPCWLEVIFNSRYPYDVPDYA

SEQ ID NO: 61 - DNA, 744 bp, Artificial Sequence
  Synthetic
ggatccggccgtaaaaaacgccgtcaacgccgccgtgaactggagagcccgccgccgccgtattctcgttacccgatggatttcctgaaaccgactgcagattgcccggacgcagtcccgtcatcggctgagaccggcggcaccaactatctggcaccgggcggtctgagtgattcccagctgctgctggaaccgggcgaccgttcacattggtgtgtggttgcctattgggaagagaaaacgcgtgtcggtcgcctgtactgcgtacaggaaccgtcgctggatatcttttatgacctgccgcagggcaatggtttctgtctgggccaactgaactcagataataaatcgcagctggtgcaaaaagttcgctcaaaaattggctgcggtatccagctgacccgtgaagttgacggtgtctgggtatataaccgcagctcttacccgatttttatcaaaagtgccaccctggataatccggactcccgtacgctgctggtccacaaagtatttccgggcttctcaatcaaagcgttcgattacgagaaagcctactcgctgcagcgcccgaacgaccatgaattcatgcagcaaccgtggacgggttttactgtgcagatctctttcgttaaaggctggggtcaatgctacacccgtcagtttatctcgtcctgtccgtgctggctggaagtgattttcaatagccgctagccatacgacgtcccagactacgcttaggtcgac

SEQ ID NO: 62 - protein, 236 aa, Artificial Sequence
  Synthetic
GSGRKKRRQRRRELESPPPPYSRYPMDFLKPTADCPDAVPSSAETGGTNYLAPGGLSDSQLLLEPGDRSHWCVVAYWEEKTRVGRLYCVQEPSLDIFYDLPQGNGFCLGQLNSDNKSQLVQKVRSKIGCGIQLTREVDGVWVYNRSSYPIFIKSATLDNPDSRTLLVHKVFPGFSIKAFDYEKAYSLQRPNDHEFMQQPWTGFTVQISFVKGWGQCYTRQFISSCPCWLEVIFNSR

SEQ ID NO: 63 - DNA, 744 bp, Artificial Sequence
  Synthetic
ggatccggccgtaaaaaacgccgtcaacgccgccgtgaactggagagcccgccgccgccgtattctcgttacccgctggatttcctgaaaccgactgcagattgcccggacgcagtcccgtcatcggctgagaccggcggcaccaactatctggcaccgggcggtctgagtgattcccagctgctgctggaaccgggcgaccgttcacattggtgtgtggttgcctattgggaagagaaaacgcgtgtcggtcgcctgtactgcgtacaggaaccgtcgctggatatcttttatgacctgccgcagggcaatggtttctgtctgggccaactgaactcagataataaatcgcagctggtgcaaaaagttcgctcaaaaattggctgcggtatccagctgacccgtgaagttgacggtgtctgggtatataaccgcagctcttacccgatttttatcaaaagtgccaccctggataatccggactcccgtacgctgctggtccacaaagtatttccgggcttctcaatcaaagcgttcgattacgagaaagcctactcgctgcagcgcccgaacgaccatgaattcatgcagcaaccgtggacgggttttactgtgcagatctctttcgttaaaggctggggtcaatgctacacccgtcagtttatctcgtcctgtccgtgctggctggaagtgattttcaatagccgctagccatacgacgtcccagactacgcttaggtcgac

SEQ ID NO: 64 - protein, 236 aa, Artificial Sequence
  Synthetic
GSGRKKRRQRRRELESPPPPYSRYPLDFLKPTADCPDAVPSSAETGGTNYLAPGGLSDSQLLLEPGDRSHWCVVAYWEEKTRVGRLYCVQEPSLDIFYDLPQGNGFCLGQLNSDNKSQLVQKVRSKIGCGIQLTREVDGVWVYNRSSYPIFIKSATLDNPDSRTLLVHKVFPGFSIKAFDYEKAYSLQRPNDHEFMQQPWTGFTVQISFVKGWGQCYTRQFISSCPCWLEVIFNSR

SEQ ID NO: 65 - DNA, 750 bp, Artificial Sequence
  Synthetic
ggatccggccgcaagaaacgccgccagcgccgccgcgaactagagtctcccccccctccttactccagatacccgatggattttctcaaaccaactgcagactgtccagatgctgtgccttcctccgctgaaacagggggaacgaattatctggcccctggggggctttcagattcccaacttcttctggagcctggggatcggtcacactggtgcgtggtggcatactgggaggagaagacgagagtggggaggctctactgtgtccaggagccctctctggatatcttctatgatctacctcaggggaatggcttttgcctcggacagctcaattcggacaacaagagtcagctggtgcagaaggtgcggagcaaaatcggctgcggcatccagctgacgcgggaggtggatggtgtgtgggtgtacaaccgcagcagttaccccatcttcatcaagtccgccacactggacaacccggactccaggacgctgttggtacacaaggtgttccccggtttctccatcaaggctttcgactacgagaaggcgtacagcctgcagcggcccaatgaccacgagtttatgcagcagccgtggacgggctttaccgtgcagatcagctttgtgaagggctggggtcagtgctacacccgccagttcatcagcagctgcccgtgctggctagaggtcatcttcaacagccgggaattctacccatacgacgtcccagactacgcttaggtcgac

SEQ ID NO: 66 - protein, 245 aa, Artificial Sequence; identical to SEQ ID NO: 58
  Synthetic

SEQ ID NO: 67 - DNA, 750 bp, Artificial Sequence
  Synthetic
ggatccggccgcaagaaacgccgccagcgccgccgcgaactagagtctcccccccctccttactccagatacccgctggattttctcaaaccaactgcagactgtccagatgctgtgccttcctccgctgaaacagggggaacgaattatctggcccctggggggctttcagattcccaacttcttctggagcctggggatcggtcacactggtgcgtggtggcatactgggaggagaagacgagagtggggaggctctactgtgtccaggagccctctctggatatcttctatgatctacctcaggggaatggcttttgcctcggacagctcaattcggacaacaagagtcagctggtgcagaaggtgcggagcaaaatcggctgcggcatccagctgacgcgggaggtggatggtgtgtgggtgtacaaccgcagcagttaccccatcttcatcaagtccgccacactggacaacccggactccaggacgctgttggtacacaaggtgttccccggtttctccatcaaggctttcgactacgagaaggcgtacagcctgcagcggcccaatgaccacgagtttatgcagcagccgtggacgggctttaccgtgcagatcagctttgtgaagggctggggtcagtgctacacccgccagttcatcagcagctgcccgtgctggctagaggtcatcttcaacagccgggaattctacccatacgacgtcccagactacgcttaggtcgac

SEQ ID NO: 68 - protein, 245 aa, Artificial Sequence; identical to SEQ ID NO: 60
  Synthetic

SEQ ID NO: 69 - DNA, 750 bp, Artificial Sequence
  Synthetic
ggatccggccgcaagaaacgccgccagcgccgccgcgaactagagtctcccccccctccttactccagatacccgatggattttctcaaaccaactgcagactgtccagatgctgtgccttcctccgctgaaacagggggaacgaattatctggcccctggggggctttcagattcccaacttcttctggagcctggggatcggtcacactggtgcgtggtggcatactgggaggagaagacgagagtggggaggctctactgtgtccaggagccctctctggatatcttctatgatctacctcaggggaatggcttttgcctcggacagctcaattcggacaacaagagtcagctggtgcagaaggtgcggagcaaaatcggctgcggcatccagctgacgcgggaggtggatggtgtgtgggtgtacaaccgcagcagttaccccatcttcatcaagtccgccacactggacaacccggactccaggacgctgttggtacacaaggtgttccccggtttctccatcaaggctttcgactacgagaaggcgtacagcctgcagcggcccaatgaccacgagtttatgcagcagccgtggacgggctttaccgtgcagatcagctttgtgaagggctggggtcagtgctacacccgccagttcatcagcagctgcccgtgctggctagaggtcatcttcaacagccggtaattctacccatacgacgtcccagactacgcttaggtcgac

SEQ ID NO: 70 - protein, 236 aa, Artificial Sequence; identical to SEQ ID NO: 62
  Synthetic

SEQ ID NO: 71 - DNA, 750 bp, Artificial Sequence
  Synthetic
ggatccggccgcaagaaacgccgccagcgccgccgcgaactagagtctcccccccctccttactccagatacccgctggattttctcaaaccaactgcagactgtccagatgctgtgccttcctccgctgaaacagggggaacgaattatctggcccctggggggctttcagattcccaacttcttctggagcctggggatcggtcacactggtgcgtggtggcatactgggaggagaagacgagagtggggaggctctactgtgtccaggagccctctctggatatcttctatgatctacctcaggggaatggcttttgcctcggacagctcaattcggacaacaagagtcagctggtgcagaaggtgcggagcaaaatcggctgcggcatccagctgacgcgggaggtggatggtgtgtgggtgtacaaccgcagcagttaccccatcttcatcaagtccgccacactggacaacccggactccaggacgctgttggtacacaaggtgttccccggtttctccatcaaggctttcgactacgagaaggcgtacagcctgcagcggcccaatgaccacgagtttatgcagcagccgtggacgggctttaccgtgcagatcagctttgtgaagggctggggtcagtgctacacccgccagttcatcagcagctgcccgtgctggctagaggtcatcttcaacagccggtaattctacccatacgacgtcccagactacgcttaggtcgac

SEQ ID NO: 72 - protein, 236 aa, Artificial Sequence; identical to SEQ ID NO: 64
  Synthetic

SEQ ID NO: 73 - DNA, 891 bp, Artificial Sequence
  Synthetic
ttcccctctagaaataattttgtttaactttaagaaggaattcaggagcccttcaccatgcgtaaaaaacgccgtcaacgccgccgtgaactggagagcccgccgccgccgtattctcgttacccgatggatttcctgaaaccgactgcagattgcccggacgcagtcccgtcatcggctgagaccggcggcaccaactatctggcaccgggcggtctgagtgattcccagctgctgctggaaccgggcgaccgttcacattggtgtgtggttgcctattgggaagagaaaacgcgtgtcggtcgcctgtactgcgtacaggaaccgtcgctggatatcttttatgacctgccgcagggcaatggtttctgtctgggccaactgaactcagataataaatcgcagctggtgcaaaaagttcgctcaaaaattggctgcggtatccagctgacccgtgaagttgacggtgtctgggtatataaccgcagctcttacccgatttttatcaaaagtgccaccctggataatccggactcccgtacgctgctggtccacaaagtatttccgggcttctcaatcaaagcgttcgattacgagaaagcctactcgctgcagcgcccgaacgaccatgaattcatgcagcaaccgtggacgggttttactgtgcagatctctttcgttaaaggctggggtcaatgctacacccgtcagtttatctcgtcctgtccgtgctggctggaagtgattttcaatagccgcaagggcgagctcaattcgaagcttgaaggtaagcctatccctaaccctctcctcggtctcgattctacgcgtaccggtcatcatcaccatcaccattgagtttgatccggctgctaacaaagcccgaaagga

SEQ ID NO: 74 - protein, 266 aa, Artificial Sequence
  Synthetic
MRKKRRQRRRELESPPPPYSRYPMDFLKPTADCPDAVPSSAETGGTNYLAPGGLSDSQLLLEPGDRSHWCVVAYWEEKTRVGRLYCVQEPSLDIFYDLPQGNGFCLGQLNSDNKSQLVQKVRSKIGCGIQLTREVDGVWVYNRSSYPIFIKSATLDNPDSRTLLVHKVFPGFSIKAFDYEKAYSLQRPNDHEFMQQPWTGFTVQISFVKGWGQCYTRQFISSCPCWLEVIFNSRKGELNSKLEGKPIPNPLLGLDSTRTGHHHHHH

SEQ ID NO: 75 - DNA, 891 bp, Artificial Sequence
  Synthetic
ttcccctctagaaataattttgtttaactttaagaaggaattcaggagcccttcaccatgcgtaaaaaacgccgtcaacgccgccgtgaactggagagcccgccgccgccgtattctcgttacccgctggatttcctgaaaccgactgcagattgcccggacgcagtcccgtcatcggctgagaccggcggcaccaactatctggcaccgggcggtctgagtgattcccagctgctgctggaaccgggcgaccgttcacattggtgtgtggttgcctattgggaagagaaaacgcgtgtcggtcgcctgtactgcgtacaggaaccgtcgctggatatcttttatgacctgccgcagggcaatggtttctgtctgggccaactgaactcagataataaatcgcagctggtgcaaaaagttcgctcaaaaattggctgcggtatccagctgacccgtgaagttgacggtgtctgggtatataaccgcagctcttacccgatttttatcaaaagtgccaccctggataatccggactcccgtacgctgctggtccacaaagtatttccgggcttctcaatcaaagcgttcgattacgagaaagcctactcgctgcagcgcccgaacgaccatgaattcatgcagcaaccgtggacgggttttactgtgcagatctctttcgttaaaggctggggtcaatgctacacccgtcagtttatctcgtcctgtccgtgctggctggaagtgattttcaatagccgcaagggcgagctcaattcgaagcttgaaggtaagcctatccctaaccctctcctcggtctcgattctacgcgtaccggtcatcatcaccatcaccattgagtttgatccggctgctaacaaagcccgaaagga

SEQ ID NO: 76 - protein, 266 aa, Artificial Sequence
  Synthetic
MRKKRRQRRRELESPPPPYSRYPLDFLKPTADCPDAVPSSAETGGTNYLAPGGLSDSQLLLEPGDRSHWCVVAYWEEKTRVGRLYCVQEPSLDIFYDLPQGNGFCLGQLNSDNKSQLVQKVRSKIGCGIQLTREVDGVWVYNRSSYPIFIKSATLDNPDSRTLLVHKVFPGFSIKAFDYEKAYSLQRPNDHEFMQQPWTGFTVQISFVKGWGQCYTRQFISSCPCWLEVIFNSRKGELNSKLEGKPIPNPLLGLDSTRTGHHHHHH

SEQ ID NO: 77 - protein, 25 aa, Artificial Sequence
  Synthetic
GRKKRRQRRRELESPPPPYSRYPMD

SEQ ID NO: 78 - protein, 15 aa, Homo sapiens
ELESPPPPYSRYPMD

SEQ ID NO: 79 - DNA, 777 bp, Artificial Sequence
  Synthetic
ggatccaaagaaacctggtgggaaacctggtggaccgaatggagccagccgaaaaagaaacgtaaagtggaactggagagcccgccgccgccgtattctcgttacccgatggatttcctgaaaccgactgcagattgcccggacgcagtcccgtcatcggctgagaccggcggcaccaactatctggcaccgggcggtctgagtgattcccagctgctgctggaaccgggcgaccgttcacattggtgtgtggttgcctattgggaagagaaaacgcgtgtcggtcgcctgtactgcgtacaggaaccgtcgctggatatcttttatgacctgccgcagggcaatggtttctgtctgggccaactgaactcagataataaatcgcagctggtgcaaaaagttcgctcaaaaattggctgcggtatccagctgacccgtgaagttgacggtgtctgggtatataaccgcagctcttacccgatttttatcaaaagtgccaccctggataatccggactcccgtacgctgctggtccacaaagtatttccgggcttctcaatcaaagcgttcgattacgagaaagcctactcgctgcagcgcccgaacgaccatgaattcatgcagcaaccgtggacgggttttactgtgcagatctctttcgttaaaggctggggtcaatgctacacccgtcagtttatctcgtcctgtccgtgctggctggaagtgattttcaatagccgctacccatacgacgtcccagactacgcttaggtcgac

SEQ ID NO: 80 - protein, 256 aa, Artificial Sequence
  Synthetic
GSKETWWETWWTEWSQPKKKRKVELESPPPPYSRYPMDFLKPTADCPDAVPSSAETGGTNYLAPGGLSDSQLLLEPGDRSHWCVVAYWEEKTRVGRLYCVQEPSLDIFYDLPQGNGFCLGQLNSDNKSQLVQKVRSKIGCGIQLTREVDGVWVYNRSSYPIFIKSATLDNPDSRTLLVHKVFPGFSIKAFDYEKAYSLQRPNDHEFMQQPWTGFTVQISFVKGWGQCYTRQFISSCPCWLEVIFNSRYPYDVPDYA

SEQ ID NO: 81 - DNA, 777 bp, Artificial Sequence
  Synthetic
ggatccaaagaaacctggtgggaaacctggtggaccgaatggagccagccgaaaaagaaacgtaaagtggaactggagagcccgccgccgccgtattctcgttacccgatggatttcctgaaaccgactgcagattgcccggacgcagtcccgtcatcggctgagaccggcggcaccaactatctggcaccgggcggtctgagtgattcccagctgctgctggaaccgggcgaccgttcacattggtgtgtggttgcctattgggaagagaaaacgcgtgtcggtcgcctgtactgcgtacaggaaccgtcgctggatatcttttatgacctgccgcagggcaatggtttctgtctgggccaactgaactcagataataaatcgcagctggtgcaaaaagttcgctcaaaaattggctgcggtatccagctgacccgtgaagttgacggtgtctgggtatataaccgcagctcttacccgatttttatcaaaagtgccaccctggataatccggactcccgtacgctgctggtccacaaagtatttccgggcttctcaatcaaagcgttcgattacgagaaagcctactcgctgcagcgcccgaacgaccatgaattcatgcagcaaccgtggacgggttttactgtgcagatctctttcgttaaaggctggggtcaatgctacacccgtcagtttatctcgtcctgtccgtgctggctggaagtgattttcaatagccgctagccatacgacgtcccagactacgcttaggtcgac

SEQ ID NO: 82 - protein, 247 aa, Artificial Sequence
  Synthetic
GSKETWWETWWTEWSQPKKKRKVELESPPPPYSRYPMDFLKPTADCPDAVPSSAETGGTNYLAPGGLSDSQLLLEPGDRSHWCVVAYWEEKTRVGRLYCVQEPSLDIFYDLPQGNGFCLGQLNSDNKSQLVQKVRSKIGCGIQLTREVDGVWVYNRSSYPIFIKSATLDNPDSRTLLVHKVFPGFSIKAFDYEKAYSLQRPNDHEFMQQPWTGFTVQISFVKGWGQCYTRQFISSCPCWLEVIFNSR

SEQ ID NO: 83 - DNA, 1425 bp, Artificial Sequence
  Synthetic
ggatccaaagaaacctggtgggaaacctggtggaccgaatggagccagccgaaaaagaaacgtaaagtgggtttccgtacgaaacgctcggccctggtccgtcgcctgtggcgctcccgtgctccgggtggtgaagatgaagaagaaggtgctggcggcggtggcggtggcggtgaactgcgtggcgagggtgcaaccgatagtcgtgcacacggtgcaggcggtggcggtccgggtcgtgctggttgctgtctgggtaaagctgtgcgcggcgcgaaaggtcatcaccatccgcacccgccggcagcaggtgcaggtgcagctggcggtgcggaagccgatctgaaagccctgacccatagtgtcctgaaaaaactgaaagaacgtcagctggagctgctgctgcaagcagtagaatcccgtggcggtacccgtacggcttgtctgctgctgccgggtcgtctggattgccgtctgggtccgggtgcaccggctggtgcgcagccggcacaaccgccgagctcttacagcctgccgctgctgctgtgtaaagtgtttcgttggccggacctgcgccacagttccgaagttaaacgcctgtgctgttgcgagagctatggcaaaattaacccggaactggtttgttgcaatccgcaccatctgtctcgtctgtgtgaactggagagcccgccgccgccgtattctcgttacccgatggatttcctgaaaccgactgcagattgcccggacgcagtcccgtcatcggctgagaccggcggcaccaactatctggcaccgggcggtctgagtgattcccagctgctgctggaaccgggcgaccgttcacattggtgtgtggttgcctattgggaagagaaaacgcgtgtcggtcgcctgtactgcgtacaggaaccgtcgctggatatcttttatgacctgccgcagggcaatggtttctgtctgggccaactgaactcagataataaatcgcagctggtgcaaaaagttcgctcaaaaattggctgcggtatccagctgacccgtgaagttgacggtgtctgggtatataaccgcagctcttacccgatttttatcaaaagtgccaccctggataatccggactcccgtacgctgctggtccacaaagtatttccgggcttctcaatcaaagcgttcgattacgagaaagcctactcgctgcagcgcccgaacgaccatgaattcatgcagcaaccgtggacgggttttactgtgcagatctctttcgttaaaggctggggtcaatgctacacccgtcagtttatctcgtcctgtccgtgctggctggaagtgattttcaatagccgcaagggcgagctcaattcgaagcttgaaggtaagcctatccctaaccctctcctcggtctcgattctacgtgagtcgac

SEQ ID NO: 84 - protein, 472 aa, Artificial Sequence
  Synthetic
GSKETWWETWWTEWSQPKKKRKVGFRTKRSALVRRLWRSRAPGGEDEEEGAGGGGGGGELRGEGATDSRAHGAGGGGPGRAGCCLGKAVRGAKGHHHPHPPAAGAGAAGGAEADLKALTHSVLKKLKERQLELLLQAVESRGGTRTACLLLPGRLDCRLGPGAPAGAQPAQPPSSYSLPLLLCKVFRWPDLRHSSEVKRLCCCESYGKINPELVCCNPHHLSRLCELESPPPPYSRYPMDFLKPTADCPDAVPSSAETGGTNYLAPGGLSDSQLLLEPGDRSHWCVVAYWEEKTRVGRLYCVQEPSLDIFYDLPQGNGFCLGQLNSDNKSQLVQKVRSKIGCGIQLTREVDGVWVYNRSSYPIFIKSATLDNPDSRTLLVHKVFPGFSIKAFDYEKAYSLQRPNDHEFMQQPWTGFTVQISFVKGWGQCYTRQFISSCPCWLEVIFNSRKGELNSKLEGKPIPNPLLGLDST

SEQ ID NO: 85 - DNA, 1356 bp, Artificial Sequence
  Synthetic
ggatccaaagaaacctggtgggaaacctggtggaccgaatggagccagccgaaaaagaaacgtaaagtgggtttccgtacgaaacgctcggccctggtccgtcgcctgtggcgctcccgtgctccgggtggtgaagatgaagaagaaggtgctggcggcggtggcggtggcggtgaactgcgtggcgagggtgcaaccgatagtcgtgcacacggtgcaggcggtggcggtccgggtcgtgctggttgctgtctgggtaaagctgtgcgcggcgcgaaaggtcatcaccatccgcacccgccggcagcaggtgcaggtgcagctggcggtgcggaagccgatctgaaagccctgacccatagtgtcctgaaaaaactgaaagaacgtcagctggagctgctgctgcaagcagtagaatcccgtggcggtacccgtacggcttgtctgctgctgccgggtcgtctggattgccgtctgggtccgggtgcaccggctggtgcgcagccggcacaaccgccgagctcttacagcctgccgctgctgctgtgtaaagtgtttcgttggccggacctgcgccacagttccgaagttaaacgcctgtgctgttgcgagagctatggcaaaattaacccggaactggtttgttgcaatccgcaccatctgtctcgtctgtgtgaactggagagcccgccgccgccgtattctcgttacccgatggatttcctgaaaccgactgcagattgcccggacgcagtcccgtcatcggctgagaccggcggcaccaactatctggcaccgggcggtctgagtgattcccagctgctgctggaaccgggcgaccgttcacattggtgtgtggttgcctattgggaagagaaaacgcgtgtcggtcgcctgtactgcgtacaggaaccgtcgctggatatcttttatgacctgccgcagggcaatggtttctgtctgggccaactgaactcagataataaatcgcagctggtgcaaaaagttcgctcaaaaattggctgcggtatccagctgacccgtgaagttgacggtgtctgggtatataaccgcagctcttacccgatttttatcaaaagtgccaccctggataatccggactcccgtacgctgctggtccacaaagtatttccgggcttctcaatcaaagcgttcgattacgagaaagcctactcgctgcagcgcccgaacgaccatgaattcatgcagcaaccgtggacgggttttactgtgcagatctctttcgttaaaggctggggtcaatgctacacccgtcagtttatctcgtcctgtccgtgctggctggaagtgattttcaatagccgctaggtcgac

SEQ ID NO: 86 - protein, 449 aa, Artificial Sequence
  Synthetic
GSKETWWETWWTEWSQPKKKRKVGFRTKRSALVRRLWRSRAPGGEDEEEGAGGGGGGGELRGEGATDSRAHGAGGGGPGRAGCCLGKAVRGAKGHHHPHPPAAGAGAAGGAEADLKALTHSVLKKLKERQLELLLQAVESRGGTRTACLLLPGRLDCRLGPGAPAGAQPAQPPSSYSLPLLLCKVFRWPDLRHSSEVKRLCCCESYGKINPELVCCNPHHLSRLCELESPPPPYSRYPMDFLKPTADCPDAVPSSAETGGTNYLAPGGLSDSQLLLEPGDRSHWCVVAYWEEKTRVGRLYCVQEPSLDIFYDLPQGNGFCLGQLNSDNKSQLVQKVRSKIGCGIQLTREVDGVWVYNRSSYPIFIKSATLDNPDSRTLLVHKVFPGFSIKAFDYEKAYSLQRPNDHEFMQQPWTGFTVQISFVKGWGQCYTRQFISSCPCWLEVIFNSR

SEQ ID NO: 87 - DNA, 1323 bp, Artificial Sequence
  Synthetic
ggatccggccgtaaaaaacgccgtcaacgccgccgtggtttccgtacgaaacgctcggccctggtccgtcgcctgtggcgctcccgtgctccgggtggtgaagatgaagaagaaggtgctggcggcggtggcggtggcggtgaactgcgtggcgagggtgcaaccgatagtcgtgcacacggtgcaggcggtggcggtccgggtcgtgctggttgctgtctgggtaaagctgtgcgcggcgcgaaaggtcatcaccatccgcacccgccggcagcaggtgcaggtgcagctggcggtgcggaagccgatctgaaagccctgacccatagtgtcctgaaaaaactgaaagaacgtcagctggagctgctgctgcaagcagtagaatcccgtggcggtacccgtacggcttgtctgctgctgccgggtcgtctggattgccgtctgggtccgggtgcaccggctggtgcgcagccggcacaaccgccgagctcttacagcctgccgctgctgctgtgtaaagtgtttcgttggccggacctgcgccacagttccgaagttaaacgcctgtgctgttgcgagagctatggcaaaattaacccggaactggtttgttgcaatccgcaccatctgtctcgtctgtgtgaactggagagcccgccgccgccgtattctcgttacccgatggatttcctgaaaccgactgcagattgcccggacgcagtcccgtcatcggctgagaccggcggcaccaactatctggcaccgggcggtctgagtgattcccagctgctgctggaaccgggcgaccgttcacattggtgtgtggttgcctattgggaagagaaaacgcgtgtcggtcgcctgtactgcgtacaggaaccgtcgctggatatcttttatgacctgccgcagggcaatggtttctgtctgggccaactgaactcagataataaatcgcagctggtgcaaaaagttcgctcaaaaattggctgcggtatccagctgacccgtgaagttgacggtgtctgggtatataaccgcagctcttacccgatttttatcaaaagtgccaccctggataatccggactcccgtacgctgctggtccacaaagtatttccgggcttctcaatcaaagcgttcgattacgagaaagcctactcgctgcagcgcccgaacgaccatgaattcatgcagcaaccgtggacgggttttactgtgcagatctctttcgttaaaggctggggtcaatgctacacccgtcagtttatctcgtcctgtccgtgctggctggaagtgattttcaatagccgctaggtcgac

SEQ ID NO: 88 - protein, 437 aa, Artificial Sequence
  Synthetic
MGRKKRRQRRRGFRTKRSALVRRLWRSRAPGGEDEEEGAGGGGGGGELRGEGATDSRAHGAGGGGPGRAGCCLGKAVRGAKGHHHPHPPAAGAGAAGGAEADLKALTHSVLKKLKERQLELLLQAVESRGGTRTACLLLPGRLDCRLGPGAPAGAQPAQPPSSYSLPLLLCKVFRWPDLRHSSEVKRLCCCESYGKINPELVCCNPHHLSRLCELESPPPPYSRYPMDFLKPTADCPDAVPSSAETGGTNYLAPGGLSDSQLLLEPGDRSHWCVVAYWEEKTRVGRLYCVQEPSLDIFYDLPQGNGFCLGQLNSDNKSQLVQKVRSKIGCGIQLTREVDGVWVYNRSSYPIFIKSATLDNPDSRTLLVHKVFPGFSIKAFDYEKAYSLQRPNDHEFMQQPWTGFTVQISFVKGWGQCYTRQFISSCPCWLEVIFNSR

SEQ ID NO: 89 - DNA, 1497 bp, Artificial Sequence
  Synthetic
  exon (58)..(1461)
ttcccctctagaaataattttgtttaactttaagaaggaattcaggagcccttcaccatgcgtaaaaaacgccgtcaacgccgccgtggtttccgtacgaaacgctcggccctggtccgtcgcctgtggcgctcccgtgctccgggtggtgaagatgaagaagaaggtgctggcggcggtggcggtggcggtgaactgcgtggcgagggtgcaaccgatagtcgtgcacacggtgcaggcggtggcggtccgggtcgtgctggttgctgtctgggtaaagctgtgcgcggcgcgaaaggtcatcaccatccgcacccgccggcagcaggtgcaggtgcagctggcggtgcggaagccgatctgaaagccctgacccatagtgtcctgaaaaaactgaaagaacgtcagctggagctgctgctgcaagcagtagaatcccgtggcggtacccgtacggcttgtctgctgctgccgggtcgtctggattgccgtctgggtccgggtgcaccggctggtgcgcagccggcacaaccgccgagctcttacagcctgccgctgctgctgtgtaaagtgtttcgttggccggacctgcgccacagttccgaagttaaacgcctgtgctgttgcgagagctatggcaaaattaacccggaactggtttgttgcaatccgcaccatctgtctcgtctgtgtgaactggagagcccgccgccgccgtattctcgttacccgatggatttcctgaaaccgactgcagattgcccggacgcagtcccgtcatcggctgagaccggcggcaccaactatctggcaccgggcggtctgagtgattcccagctgctgctggaaccgggcgaccgttcacattggtgtgtggttgcctattgggaagagaaaacgcgtgtcggtcgcctgtactgcgtacaggaaccgtcgctggatatcttttatgacctgccgcagggcaatggtttctgtctgggccaactgaactcagataataaatcgcagctggtgcaaaaagttcgctcaaaaattggctgcggtatccagctgacccgtgaagttgacggtgtctgggtatataaccgcagctcttacccgatttttatcaaaagtgccaccctggataatccggactcccgtacgctgctggtccacaaagtatttccgggcttctcaatcaaagcgttcgattacgagaaagcctactcgctgcagcgcccgaacgaccatgaattcatgcagcaaccgtggacgggttttactgtgcagatctctttcgttaaaggctggggtcaatgctacacccgtcagtttatctcgtcctgtccgtgctggctggaagtgattttcaatagccgcaagggcgagctcaattcgaagcttgaaggtaagcctatccctaaccctctcctcggtctcgattctacgcgtaccggtcatcatcaccatcaccattgagtttgatccggctgctaacaaagcccgaaagga

SEQ ID NO: 90 - protein, 468 aa, Artificial Sequence
  Synthetic
MRKKRRQRRRGFRTKRSALVRRLWRSRAPGGEDEEEGAGGGGGGGELRGEGATDSRAHGAGGGGPGRAGCCLGKAVRGAKGHHHPHPPAAGAGAAGGAEADLKALTHSVLKKLKERQLELLLQAVESRGGTRTACLLLPGRLDCRLGPGAPAGAQPAQPPSSYSLPLLLCKVFRWPDLRHSSEVKRLCCCESYGKINPELVCCNPHHLSRLCELESPPPPYSRYPMDFLKPTADCPDAVPSSAETGGTNYLAPGGLSDSQLLLEPGDRSHWCVVAYWEEKTRVGRLYCVQEPSLDIFYDLPQGNGFCLGQLNSDNKSQLVQKVRSKIGCGIQLTREVDGVWVYNRSSYPIFIKSATLDNPDSRTLLVHKVFPGFSIKAFDYEKAYSLQRPNDHEFMQQPWTGFTVQISFVKGWGQCYTRQFISSCPCWLEVIFNSRKGELNSKLEGKPIPNPLLGLDSTRTGHHHHHH

SEQ ID NO: 91 - protein, 461 aa, Artificial Sequence
  Synthetic
GSGRKKRRQRRRGFRTKRSALVRRLWRSRAPGGEDEEEGAGGGGGGGELRGEGATDSRAHGAGGGGPGRAGCCLGKAVRGAKGHHHPHPPAAGAGAAGGAEADLKALTHSVLKKLKERQLELLLQAVESRGGTRTACLLLPGRLDCRLGPGAPAGAQPAQPPSSYSLPLLLCKVFRWPDLRHSSEVKRLCCCESYGKINPELVCCNPHHLSRLCELESPPPPYSRYPMDFLKPTADCPDAVPSSAETGGTNYLAPGGLSDSQLLLEPGDRSHWCVVAYWEEKTRVGRLYCVQEPSLDIFYDLPQGNGFCLGQLNSDNKSQLVQKVRSKIGCGIQLTREVDGVWVYNRSSYPIFIKSATLDNPDSRTLLVHKVFPGFSIKAFDYEKAYSLQRPNDHEFMQQPWTGFTVQISFVKGWGQCYTRQFISSCPCWLEVIFNSRKGELNSKLEGKPIPNPLLGLDST

SEQ ID NO: 92 - DNA, 1392 bp, Artificial Sequence
  Synthetic
ggatccggccgtaaaaaacgccgtcaacgccgccgtggtttccgtacgaaacgctcggccctggtccgtcgcctgtggcgctcccgtgctccgggtggtgaagatgaagaagaaggtgctggcggcggtggcggtggcggtgaactgcgtggcgagggtgcaaccgatagtcgtgcacacggtgcaggcggtggcggtccgggtcgtgctggttgctgtctgggtaaagctgtgcgcggcgcgaaaggtcatcaccatccgcacccgccggcagcaggtgcaggtgcagctggcggtgcggaagccgatctgaaagccctgacccatagtgtcctgaaaaaactgaaagaacgtcagctggagctgctgctgcaagcagtagaatcccgtggcggtacccgtacggcttgtctgctgctgccgggtcgtctggattgccgtctgggtccgggtgcaccggctggtgcgcagccggcacaaccgccgagctcttacagcctgccgctgctgctgtgtaaagtgtttcgttggccggacctgcgccacagttccgaagttaaacgcctgtgctgttgcgagagctatggcaaaattaacccggaactggtttgttgcaatccgcaccatctgtctcgtctgtgtgaactggagagcccgccgccgccgtattctcgttacccgatggatttcctgaaaccgactgcagattgcccggacgcagtcccgtcatcggctgagaccggcggcaccaactatctggcaccgggcggtctgagtgattcccagctgctgctggaaccgggcgaccgttcacattggtgtgtggttgcctattgggaagagaaaacgcgtgtcggtcgcctgtactgcgtacaggaaccgtcgctggatatcttttatgacctgccgcagggcaatggtttctgtctgggccaactgaactcagataataaatcgcagctggtgcaaaaagttcgctcaaaaattggctgcggtatccagctgacccgtgaagttgacggtgtctgggtatataaccgcagctcttacccgatttttatcaaaagtgccaccctggataatccggactcccgtacgctgctggtccacaaagtatttccgggcttctcaatcaaagcgttcgattacgagaaagcctactcgctgcagcgcccgaacgaccatgaattcatgcagcaaccgtggacgggttttactgtgcagatctctttcgttaaaggctggggtcaatgctacacccgtcagtttatctcgtcctgtccgtgctggctggaagtgattttcaatagccgcaagggcgagctcaattcgaagcttgaaggtaagcctatccctaaccctctcctcggtctcgattctacgtgagtcgac

SEQ ID NO: 93 - DNA, 618 bp, Artificial Sequence
  Synthetic
  exon (1)..(609)
ggatccggccgtaaaaaacgccgtcaacgccgccgttcacattggtgtgtggttgcctattgggaagagaaaacgcgtgtcggtcgcctgtactgcgtacaggaaccgtcgctggatatcttttatgacctgccgcagggcaatggtttctgtctgggccaactgaactcagataataaatcgcagctggtgcaaaaagttcgctcaaaaattggctgcggtatccagctgacccgtgaagttgacggtgtctgggtatataaccgcagctcttacccgatttttatcaaaagtgccaccctggataatccggactcccgtacgctgctggtccacaaagtatttccgggcttctcaatcaaagcgttcgattacgagaaagcctactcgctgcagcgcccgaacgaccatgaattcatgcagcaaccgtggacgggttttactgtgcagatctctttcgttaaaggctggggtcaatgctacacccgtcagtttatctcgtcctgtccgtgctggctggaagtgattttcaatagccgcaagggcgagctcaattcgaagcttgaaggtaagcctatccctaaccctctcctcggtctcgattctacgtgagtcgac

SEQ ID NO: 94 - protein, 203 aa, Artificial Sequence
  Synthetic
GSGRKKRRQRRRSHWCVVAYWEEKTRVGRLYCVQEPSLDIFYDLPQGNGFCLGQLNSDNKSQLVQKVRSKIGCGIQLTREVDGVWVYNRSSYPIFIKSATLDNPDSRTLLVHKVFPGFSIKAFDYEKAYSLQRPNDHEFMQQPWTGFTVQISFVKGWGQCYTRQFISSCPCWLEVIFNSRKGELNSKLEGKPIPNPLLGLDST

SEQ ID NO: 95 - DNA, 1392 bp, Artificial Sequence
  Synthetic
  exon (1)..(1383)
ggatccggccgtaaaaaacgccgtcaacgccgccgtggtttccgtacgaaacgcagcgccctggtccgtcgcctgtggcgcagccgtgctccgggtggtgaagatgaagaagaaggtgctggcggcggtggcggtggcggtgaactgcgtggcgagggtgcaaccgatagccgtgcacatggtgcaggcggtggcggtccgggtcgtgctggttgctgtctgggtaaagctgtgcgcggcgcgaaaggtcatcatcatccgcatccgccggcagcaggtgcaggtgcagctggcggtgcggaagccgatctgaaagccctgacccatagcgtcctgaaaaaactgaaagaacgtcagctggagctgctgctgcaagcagtagaaagccgtggcggtacccgtacggcttgtctgctgctgccgggtcgtctggattgccgtctgggtccgggtgcaccggctggtgcgcagccggcacaaccgccgagcagctacagcctgccgctgctgctgtgtaaagtgtttcgttggccggacctgcgccatagcagcgaagttaaacgcctgtgctgttgcgagagctatggcaaaattaacccggaactggtttgttgcaatccgcatcatctgagccgtctgtgtgaactggagagcccgccgccgccgtatagccgttacccgctggatttcctgaaaccgactgcagattgcccggacgcagtcccgagcagcgctgagaccggcggcaccaactatctggcaccgggcggtctgagcgatagccagctgctgctggaaccgggcgaccgtagccattggtgtgtggttgcctattgggaagagaaaacgcgtgtcggtcgcctgtactgcgtacaggaaccgagcctggatatcttttatgacctgccgcagggcaatggtttctgtctgggccaactgaacagcgataataaaagccagctggtgcaaaaagttcgcagcaaaattggctgcggtatccagctgacccgtgaagttgacggtgtctgggtatataaccgcagcagctacccgatttttatcaaaagcgccaccctggataatccggacagccgtacgctgctggtccataaagtatttccgggcttcagcatcaaagcgttcgattacgagaaagcctacagcctgcagcgcccgaacgaccatgaattcatgcagcaaccgtggacgggttttactgtgcagatcagcttcgttaaaggctggggtcaatgctacacccgtcagtttatcagcagctgtccgtgctggctggaagtgattttcaatagccgcaagggcgagctcaatagcaagcttgaaggtaagcctatccctaaccctctcctcggtctcgatagcacgtgagtcgac

SEQ ID NO: 96 - protein, 461 aa, Artificial Sequence
  Synthetic
GSGRKKRRQRRRGFRTKRSALVRRLWRSRAPGGEDEEEGAGGGGGGGELRGEGATDSRAHGAGGGGPGRAGCCLGKAVRGAKGHHHPHPPAAGAGAAGGAEADLKALTHSVLKKLKERQLELLLQAVESRGGTRTACLLLPGRLDCRLGPGAPAGAQPAQPPSSYSLPLLLCKVFRWPDLRHSSEVKRLCCCESYGKINPELVCCNPHHLSRLCELESPPPPYSRYPLDFLKPTADCPDAVPSSAETGGTNYLAPGGLSDSQLLLEPGDRSHWCVVAYWEEKTRVGRLYCVQEPSLDIFYDLPQGNGFCLGQLNSDNKSQLVQKVRSKIGCGIQLTREVDGVWVYNRSSYPIFIKSATLDNPDSRTLLVHKVFPGFSIKAFDYEKAYSLQRPNDHEFMQQPWTGFTVQISFVKGWGQCYTRQFISSCPCWLEVIFNSRKGELNSKLEGKPIPNPLLGLDST

SEQ ID NO: 97 - DNA, 558 bp, Artificial Sequence
  Synthetic
ggatccggccgtaaaaaacgccgtcaacgccgccgttcacattggtgtgtggttgcctattgggaagagaaaacgcgtgtcggtcgcctgtactgcgtacaggaaccgagcctggatatcttttatgacctgccgcagggcaatggtttctgtctgggccaactgaacagcgataataaaagccagctggtgcaaaaagttcgcagcaaaattggctgcggtatccagctgacccgtgaagttgacggtgtctgggtatataaccgcagcagctacccgatttttatcaaaagcgccaccctggataatccggacagccgtacgctgctggtccataaagtatttccgggcttcagcatcaaagcgttcgattacgagaaagcctacagcctgcagcgcccgaacgaccatgaattcatgcagcaaccgtggacgggttttactgtgcagatcagcttcgttaaaggctggggtcaatgcaagggcgagctcaatagcaagcttgaaggtaagcctatccctaaccctctcctcggtctcgatagcacgtgagtcgac

SEQ ID NO: 98 - DNA, 618 bp, Artificial Sequence
  Synthetic
  exon (1)..(609)
ggatccggccgtaaaaaacgccgtcaacgccgccgttcacattggtgtgtggttgcctattgggaagagaaaacgcgtgtcggtcgcctgtactgcgtacaggaaccgagcctggatatcttttatgacctgccgcagggcaatggtttctgtctgggccaactgaacagcgataataaaagccagctggtgcaaaaagttcgcagcaaaattggctgcggtatccagctgacccgtgaagttgacggtgtctgggtatataaccgcagcagctacccgatttttatcaaaagcgccaccctggataatccggacagccgtacgctgctggtccataaagtatttccgggcttcagcatcaaagcgttcgattacgagaaagcctacagcctgcagcgcccgaacgaccatgaattcatgcagcaaccgtggacgggttttactgtgcagatcagcttcgttaaaggctggggtcaatgctacacccgtcagtttatcagcagctgtccgtgctggctggaagtgattttcaatagccgcaagggcgagctcaatagcaagcttgaaggtaagcctatccctaaccctctcctcggtctcgatagcacgtgagtcgac

SEQ ID NO: 99 - DNA, 1365 bp, Artificial Sequence
  Synthetic
  exon (1)..(1356)
ggatccggtcgtaaaaaacgtcgtcagcgtcgtcgtggtttccgtaccaaacgttctgcgctggttcgtcgtctgtggcgttctcgtgcgccgggtggtgaagacgaagaagaaggtgcgggtggtggtggtggtggtggtgaactgcgtggtgaaggtgcgaccgactctcgtgcgcacggtgcgggtggtggtggtccgggtcgtgcgggttgctgcctgggtaaagcggttcgtggtgcgaaaggtcaccaccacccgcacccgccggcggcgggtgcgggtgcggcgggtggtgcggaagcggacctgaaagcgctgacccactctgttctgaaaaaactgaaagaacgtcagctggaactgctgctgcaggcggttgaatctcgtggtggtacccgtaccgcgtgcctgctgctgccgggtcgtctggactgccgtctgggtccgggtgcgccggcgggtgcgcagccggcgcagccgccgtcttcttactctctgccgctgctgctgtgcaaagttttccgttggccggacctgcgtcactcttctgaagttaaacgtctgtgctgctgcgaatcttacggtaaaatcaacccggaactggtttgctgcaacccgcaccacctgtctcgtctgtgcgaactggaatctccgccgccgccgtactctcgttacccgctggacttcctgaaaccgaccgcggactgcccggacgcggttccgtcttctgcggaaaccggtggtaccaactacctggcgccgggtggtctgtctgactctcagctgctgctggaaccgggtgaccgttctcactggtgcgttgttgcgtactgggaagaaaaaacccgtgttggtcgtctgtactgcgttcaggaaccgtctctggacatcttctacgacctgccgcagggtaacggtttctgcctgggtcagctgaactctgacaacaaatctcagctggttcagaaagttcgttctaaaatcggttgcggtatccagctgacccgtgaagttgacggtgtttgggtttacaaccgttcttcttacccgatcttcatcaaatctgcgaccctggacaacccggactctcgtaccctgctggttcacaaagttttcccgggtttctctatcaaagcgttcgactacgaaaaagcgtactctctgcagcgtccgaacgaccacgaattcatgcagcagccgtggaccggtttcaccgttcagatctctttcgttaaaggttggggtcagtgctacacccgtcagttcatctcttcttgcccgtgctggctggaagttatcttcaactctcgtggtaaaccgatcccgaacccgctgctgggtctggactctacctgagtcgac

SEQ ID NO: 100 - protein, 452 aa, Artificial Sequence
  Synthetic
GSGRKKRRQRRRGFRTKRSALVRRLWRSRAPGGEDEEEGAGGGGGGGELRGEGATDSRAHGAGGGGPGRAGCCLGKAVRGAKGHHHPHPPAAGAGAAGGAEADLKALTHSVLKKLKERQLELLLQAVESRGGTRTACLLLPGRLDCRLGPGAPAGAQPAQPPSSYSLPLLLCKVFRWPDLRHSSEVKRLCCCESYGKINPELVCCNPHHLSRLCELESPPPPYSRYPLDFLKPTADCPDAVPSSAETGGTNYLAPGGLSDSQLLLEPGDRSHWCVVAYWEEKTRVGRLYCVQEPSLDIFYDLPQGNGFCLGQLNSDNKSQLVQKVRSKIGCGIQLTREVDGVWVYNRSSYPIFIKSATLDNPDSRTLLVHKVFPGFSIKAFDYEKAYSLQRPNDHEFMQQPWTGFTVQISFVKGWGQCYTRQFISSCPCWLEVIFNSRGKPIPNPLLGLDST

SEQ ID NO: 101 - DNA, 1365 bp, Artificial Sequence; identical to SEQ ID NO: 99
  Synthetic

SEQ ID NO: 102 - DNA, 1392 bp, Artificial Sequence; identical to SEQ ID NO: 95
  Synthetic

SEQ ID NO: 103 - DNA, 2182 bp, Artificial Sequence
  Synthetic
gacgttgtatacgactcctatagggcggccgggaattcgtcgactggatccggtaccgaggagatctgccgccgcgatcgccatgtcccccattctgggctactggaagattaagggcctggtgcagcctactagactgctgctggaatacctggaggaaaaatatgaagagcatctgtatgaaagagacgagggggataaatggaggaacaagaaattcgaactgggactggagtttcctaatctgccatactatattgacggcgatgtgaagctgactcagtctatggctatcattagatacatcgcagacaaacacaacatgctgggcgggtgtcctaaggaaagggcagagattagtatgctggagggagccgtgctggatattagatacggcgtctcacgcatcgcctatagcaaagacttcgaaaccctgaaggtggattttctgagcaaactgcctgaaatgctgaagatgttcgaggacagactgtgccacaaaacctacctgaatggcgaccatgtcacacacccagattttatgctgtacgacgccctggatgtggtcctgtatatggaccccatgtgtctggatgctttccctaagctggtgtgctttaagaaaaggatcgaggcaattccccagatcgataagtacctgaaaagctccaagtatatcgcttggcctctccagggctggcaggcaacattcggaggcggggaccatccccctaaaagcgacctggaggtgctgtttcagggaccactgggcagcggccggaagaagcggcggcagaggcgccgaagtaggttccgcactaagcggtcagcactggtgcggagactgtggcgatctcgggctcctggaggagaggacgaggaagagggagcaggcggcggcggaggaggaggagaactgcgcggggagggagctacagatagccgagcccacggagctggaggaggaggaccagggcgagccggatgctgtctgggcaaagcagtgagaggcgccaaggggcaccatcacccccatccacccgccgctggcgcaggagcagccggcggagctgaggcagacctgaaagccctgactcacagtgtgctgaagaaactgaaggaaagacagctggagctgctgctccaggcagtcgaatcacgcggaggcacccgaacagcttgtctgctgctgcccggccggctggactgccggctgggacccggcgcccctgctggggcacagccagcccagcctccatctagttatagcctgcccctgctgctgtgtaaggtgttccgatggcctgatctgcggcattcaagcgaagtcaaaaggctgtgctgttgcgagtcctacggcaagattaacccagaactggtgtgttgcaatccccatcacctgtctcgactgtgtgaactggagtccccccctccaccctactctaggtatcctatggactttctgaagccaaccgctgactgcccagatgcagtgccctcctctgccgagactgggggaaccaactacctggctcctggcggactgagcgactcccagctgctgctggaaccaggggatcgcagccactggtgtgtggtcgcctactgggaagagaagacaagagtgggaaggctgtattgcgtccaggagccttccctggacatcttctacgatctgccacaggggaatggattttgtctgggccagctgaactctgacaataagagtcagctggtgcagaaagtccggagcaagattggctgcggcatccagctgaccagggaggtggacggcgtgtgggtctacaaccgcagttcatatccaatcttcatcaagagcgccactctggacaatcccgattcccgcaccctgctggtgcataaggtcttccccggcttcagcatcaaggccttcgactacgagaaggcttatagtctccagcggcccaacgatcacgagttcatgcagcagccttggacaggcttcactgtgcagatcagcttcgtcaagggatggggccagtgctacacaaggcagttcatctcaagctgtccctgttggctggaagtcattttcaatagtaggacgcgtacgcggccgctcgagcagaaactcatctcagaagaggatctggcagcaaatgatatcctggattacaaggatgacgacgataaggtttaa

SEQ ID NO: 104 - DNA, 1053 bp, Artificial Sequence
  Synthetic
tcacccccatccacccgccgctggcgcaggagcagccggcggagctgaggcagacctgaaagccctgactcacagtgtgctgaagaaactgaaggaaagacagctggagctgctgctccaggcagtcgaatcacgcggaggcacccgaacagcttgtctgctgctgcccggccggctggactgccggctgggacccggcgcccctgctggggcacagccagcccagcctccatctagttatagcctgcccctgctgctgtgtaaggtgttccgatggcctgatctgcggcattcaagcgaagtcaaaaggctgtgctgttgcgagtcctacggcaagattaacccagaactggtgtgttgcaatccccatcacctgtctcgactgtgtgaactggagtccccccctccaccctactctaggtatcctatggactttctgaagccaaccgctgactgcccagatgcagtgccctcctctgccgagactgggggaaccaactacctggctcctggcggactgagcgactcccagctgctgctggaaccaggggatcgcagccactggtgtgtggtcgcctactgggaagagaagacaagagtgggaaggctgtattgcgtccaggagccttccctggacatcttctacgatctgccacaggggaatggattttgtctgggccagctgaactctgacaataagagtcagctggtgcagaaagtccggagcaagattggctgcggcatccagctgaccagggaggtggacggcgtgtgggtctacaaccgcagttcatatccaatcttcatcaagagcgccactctggacaatcccgattcccgcaccctgctggtgcataaggtcttccccggcttcagcatcaaggccttcgactacgagaaggcttatagtctccagcggcccaacgatcacgagttcatgcagcagccttggacaggcttcactgtgcagatcagcttcgtcaagggatggggccagtgctacacaaggcagttcatctcaagctgtccctgttggctggaagtcattttcaa

SEQ ID NO: 105 - DNA, 1370 bp, Artificial Sequence
  Synthetic
gaattcgtcgactggatccggtaccgaggagatctgccgccgcgatcgccatgtcccccattctgggctactggaagattaagggcctggtgcagcctactagactgctgctggaatacctggaggaaaaatatgaagagcatctgtatgaaagagacgagggggataaatggaggaacaagaaattcgaactgggactggagtttcctaatctgccatactatattgacggcgatgtgaagctgactcagtctatggctatcattagatacatcgcagacaaacacaacatgctgggcgggtgtcctaaggaaagggcagagattagtatgctggagggagccgtgctggatattagatacggcgtctcacgcatcgcctatagcaaagacttcgaaaccctgaaggtggattttctgagcaaactgcctgaaatgctgaagatgttcgaggacagactgtgccacaaaacctacctgaatggcgaccatgtcacacacccagattttatgctgtacgacgccctggatgtggtcctgtatatggaccccatgtgtctggatgctttccctaagctggtgtgctttaagaaaaggatcgaggcaattccccagatcgataagtacctgaaaagctccaagtatatcgcttggcctctccagggctggcaggcaacattcggaggcggggaccatccccctaaaagcgacctggaggtgctgtttcagggaccactgggcagcggccggaagaagcggcggcagaggcgcagccactggtgtgtggtcgcctactgggaagagaagacaagagtgggaaggctgtattgcgtccaggagccttccctggacatcttctacgatctgccacaggggaatggattttgtctgggccagctgaactctgacaataagagtcagctggtgcagaaagtccggagcaagattggctgcggcatccagctgaccagggaggtggacggcgtgtgggtctacaaccgcagttcatatccaatcttcatcaagagcgccactctggacaatcccgattcccgcaccctgctggtgcataaggtcttccccggcttcagcatcaaggccttcgactacgagaaggcttatagtctccagcggcccaacgatcacgagttcatgcagcagccttggacaggcttcactgtgcagatcagcttcgtcaagggatggggccagtgctacacaaggcagttcatctcaagctgtccctgttggctggaagtcattttcaatagtaggacgcgtacgcggccgctcgagcagaaactcatctcagaagaggatctggcagcaaatgatatcctggattacaaggatgacgacgataaggtttaa

SEQ ID NO: 106 - protein, 13 aa, Artificial Sequence
  Synthetic
GLPIPNPLLGLDS

(19) United States
(12) Patent Application Publication
(10) Pub. No.: US 2020/0332302 A1
(43) Pub. Date: Oct. 22, 2020

(54) THERAPEUTICS OF PTD-SMAD7 FUSION PROTEINS

(71) Applicant: THE REGENTS OF THE UNIVERSITY OF COLORADO, a body corporate, Denver, CO (US)

(72) Inventor: Xiao-Jing WANG, Greenwood Village, CO (US)

(21) Appl. No.: 16/959,252

(22) PCT Filed: Dec. 30, 2018
(86) PCT No.: PCT/US2018/068088

§ 371 Date: Jun. 30, 2020

Related U.S. Application Data
(60) Provisional application No. 62/612,439, filed on Dec. 30, 2017.

(51) Int. Cl. C12N 15/62; C12N 15/79; C07K 14/495; A61P 37/02; A61P 17/02

(52) U.S. Cl. CPC C12N 15/62; C12N 15/79; A61K 38/00; A61P 37/02; A61P 17/02; C07K 14/495

(57) ABSTRACT

The present technology provides methods and compositions for the treatment of inflammatory and/or tissue damage conditions. In particular, the use of truncated Smad7 compositions delivered locally or systemically to a site of inflammation and/or tissue damage is described. Other specific embodiments concern treatment or prevention of side effects caused by radiation and/or chemotherapy, including but not limited to oral and gastric mucositis. Also provided are codon-optimized nucleic acids encoding for Smad7 fusion proteins.

### CROSS REFERENCE TO RELATED APPLICATION

This application claims the benefit of U.S. Provisional Patent Application Ser. No. 62/612,439, filed Dec. 30, 2017, which is incorporated herein by reference.

### STATEMENT OF GOVERNMENT INTEREST

This invention was made with government support under grant numbers R44DE024659 and R44DE028718 awarded by the National Institutes of Health. The U.S. government has certain rights in the invention.

### BACKGROUND

Oral mucositis, a severe oral ulceration, is a common adverse effect of a large dose of radiation for bone marrow transplant or craniofacial radiotherapy for cancer. Severe oral mucositis could require feeding tubes, management of severe pain, and prematurely halting radiotherapy. Excessive inflammation and epithelial ablation are key features of oral mucositis.

Palifermin, a KGF (human keratinocyte growth factor) recombinant protein, is approved for preventing oral mucositis in bone-marrow transplant patients. Two Palifermin clinical trials in head and neck cancer patients showed that Palifermin reduced severe oral mucositis incidence from 67% and 69% to 51% and 54%, respectively. Other oral mucositis drugs in clinical trials or pre-clinical studies include growth factors, agents for radioprotection, anti-inflammatory agents or immune modulators.

The modest effects of Palifermin and drugs being developed in the above-mentioned categories highlight the need for identification of biomarkers for novel therapies. However, the lack of routine diagnostic biopsies or discarded tissues from oral mucositis patients has hindered this effort.

Cutaneous wound healing progresses through three overlapping phases: inflammation, tissue formation, and tissue remodeling. These are dynamic processes that involve interactions among the epidermis, leukocytes, extracellular matrix (ECM), and dermal fibroblasts. In response to skin injury, blood clots, infiltrated inflammatory cells and other cell types in the wound release multiple cytokines and chemokines. These cytokines initiate fibroblast proliferation and synthesis of ECM that fill the wound deficit and lead to wound closure.

Meanwhile, keratinocytes at the wound edge begin to proliferate and migrate to cover the wound surface. Underneath the re-epithelialized epidermis, new stroma, called granulation tissue, begins to fill the wound space, which contains provisional ECM, inflammatory cells, fibroblasts, and blood vessels. Once the wound area is filled with the granulation tissue and covered by newly re-epithelialized epidermis, the process of wound closure is completed. Later on, the wound gradually returns to normal strength and texture through tissue remodeling.

Injury-associated fibrotic diseases include burn-induced (chemical included) skin and soft tissue scarring and contraction, radiation-induced skin and organ scarring, post cancer therapeutic radiation treatment, and keloids (skin).

Severe radiodermatitis causes skin erosion, ulceration, opiate-resistant pain, and can be dose-limiting for cancer therapy. Radiodermatitis incidence and severity are increasing with the rise of the cancer population and with the use of targeted therapies (e.g., immunotherapy) in combination with radiation therapy (RT). The complexity of radiodermatitis (a combination of RT damage, dermatitis and ulceration) makes it difficult to treat. Currently, other than symptom control, there is no treatment to promote healing of radiodermatitis.

Radiation-induced lung disease (RILD) is a frequent complication of radiotherapy to the chest for chest wall or intrathoracic malignancies and can have a variety of appearances, especially depending on when the patient is imaged. Acute and late phases are described, corresponding to radiation pneumonitis and radiation fibrosis respectively. These occur at different times after completion of radiotherapy and have different imaging features and differential diagnoses. Subjects having a radiation-induced lung disorder (e.g., radiation pneumonitis) typically present with a nonproductive cough, shortness of breath, and low-grade fever. Upon evaluation, subjects are found to have reduced total lung volume, residual volume, and vital capacity, but unrestricted air flow into and out of the lungs.

Acute fibrosis (usually with a sudden and severe onset and of short duration) occurs as a common response to various forms of trauma including accidental injuries (particularly injuries to the spine and central nervous system), infections, surgery, ischemic illness (e.g. cardiac scarring following heart attack), burns, environmental pollutants, alcohol and other types of toxins, acute respiratory distress syndrome, radiation, and chemotherapy treatment.

Contraction of collagen-including tissue, which may also include other extracellular matrix components, frequently occurs in the healing of burns. The burns may be chemical, thermal, or radiation burns and may be of the eye, the surface of the skin, or the skin and the underlying tissues. It may also be the case that there are burns on internal tissues, for example, caused by radiation treatment. Contraction of burnt tissues is often a problem and may lead to physical and/or cosmetic problems, for example, loss of movement and/or disfigurement.

In the lung, aberrant wound healing responses to injury contribute to the pathogenesis of fibrotic lung diseases. Fibrotic lung diseases, such as idiopathic pulmonary fibrosis (IPF), are associated with high morbidity and mortality.

Among the many molecules known to influence wound healing, transforming growth factor β (TGF-β) has the broadest spectrum of action, affecting all cell types that are involved in all stages of wound healing (Feng et al., Annu Rev Cell Dev Biol 21:659-693, 2005). The various functions of TGF-β are mediated by a number of signaling molecules, including the Smad family members. When a ligand binds to TGF-β type I and type II receptors (TGFβRI and TGF-βRII), TGF-βRI phosphorylates Smad2 and Smad3. Phosphorylated Smad2 and Smad3 bind a co-Smad, Smad4, to form heteromeric Smad complexes and translocate into the nucleus to regulate transcription of TGF-β target genes.

TGF-β signaling has been reported to exert both positive and negative effects on wound healing (Wang et al., J Investig Dermatol Symp Proc 11: 112-117, 2006). For instance, Smad3 deficient mice, in which TGF-β signaling is partially abrogated, exhibit accelerated wound healing (Ashcroft et al., Nat Cell Biol 1:260-266, 1999). In contrast, the introduction of exogenous Smad3 to wound sites to enhance TGF-β signaling also accelerated wound healing in a rabbit dermal ulcer model (Sumiyoshi et al., J Invest Dermatol 123:229-236, 2004). Skin wounds in Smad4-deficient mice have a dramatic increase in inflammation and angiogenesis causing a delay in wound closure and formed an excessive scar (Owens et al., Am J Pathol 176:122-133, 2010). Transient adenoviral gene transfer of Smad7, an antagonist of TGF-β signaling, in corneal epithelium and stroma resulted in accelerated corneal wound healing with reduced inflammation (Saika et al., Am J Pathol 166:1405-1418, 2005). Further, Smad7 gene transfer to the lens epithelium and stroma prevented injury-induced epithelial-mesenchymal transition of lens epithelial cells and suggests a potential role of Smad7 in prevention of capsular fibrosis (Saika et al., Lab Invest 84:1259-1270, 2004). However, adenoviral vector delivery of Smad7 to balloon injury in rat carotid arteries resulted in reduced vascular healing (Mallawaarachchi et al., Arterioscler Thromb Vasc Biol 25: 1383-1387, 2005). These studies suggest that the effects of TGF-β signaling components, such as Smad7, on wound healing are complex and highly context-specific. Additionally, the effect of Smad7 may not always be explained by its role in TGF-β signaling. For instance, Smad7 has been shown to interact with components of Wnt/β-catenin (Han et al., Dev Cell Biol 11:301-312, 2006) and TNFβ/NF-κB (Hong et al., Nat Immunol 8:504-513, 2007) families.

### SUMMARY

The present technology provides a nucleic acid molecule comprising a codon-optimized human Smad7 cDNA nucleotide sequence, such as Tat-PY-C-Smad7 fusion protein (FIGS. 1A and 1B). In some embodiments, the codon-optimized human Smad7 nucleotide sequence may include one or more codons for arginine optimized for expression in one or more of bacteria or yeast, including one or more codons for serine optimized for expression in one or more of bacteria or yeast, and/or including one or more codons for histidine optimized for expression in one or more of bacteria or yeast. In some embodiments, the codon-optimized human Smad7 nucleotide sequence may include 28 serine codons, 6 histidine codons, and 9 arginine codons optimized for expression in one or more of bacteria or yeast. In some embodiments, the human Smad7 amino acid sequence may comprise or consist of an amino acid sequence set forth in any one of SEQ ID NOs: 58, 60, 62, 64, 66, 68, 70, 72, 74, 76, 80, 82, 84, 86, 88, 90, 91, 94, 96, and 100. In some embodiments, the Smad7 nucleotide sequence may be any one of the nucleotide sequences in SEQ ID NOs: 57, 59, 61, 63, 65, 67, 69, 71, 73, 75, 79, 81, 83, 85, 87, 89, 92, 93, 95, 97, 98, 99, 101, 102, 103, 104, and 105. In some embodiments, the codon-optimized human Smad7 nucleotide sequence may be in any one of the nucleotide sequences set forth in SEQ ID NOs: 57, 59, 90, 92, 93, 95, 98, 99, 101, 102, 103, 104, and 105. In some embodiments, the codon-optimized human Smad7 nucleotide sequence may have about 65 to 75 percent homology to human Smad7 cDNA, may comprise a nucleotide sequence encoding an N-terminal fragment SMAD7, may comprise a nucleotide sequence encoding a C-terminal fragment of SMAD7, or may comprise nucleotides encoding amino acids 203-426 of the human Smad7 protein, or may comprise any Smad7 protein, or fragment thereof (or nucleotide sequence endocing the same) selected from any of the sequences set forth at pages 81-134 of this disclosure. In some embodiments, any of the foregoing may further comprise a nucleotide sequence encoding a protein transduction domain, such as Tat. In some embodiments, any of the foregoing may also further comprise a nucleotide sequence encoding one or more of an epitope tag or a purification tag, such as V5, glutathione-S-transferase, or 6-Histidine (SEQ ID NO: 56).

In some embodiments, any of the foregoing may be isolated and/or purified. In some embodiments, any one of the foregoing may also encode a polypeptide having one or more biological activities selected from the group consisting of reducing or eliminating phosphorylation of Smad2, reducing or eliminating nuclear translocation of the NF-κB p50 subunit, increasing cell proliferation, reducing apoptosis, reducing radiation-induced DNA damage, reducing inflammation, reducing angiogenesis, promoting healing in oral mucositis, promoting wound healing, and treating auto-immune disease. In some embodiments, pharmaceutical compositions comprising the nucleic acid molecules above and one or more pharmaceutically acceptable excipients are provided. In some embodiments, expression vectors comprising the nucleic acid molecules above operably linked to a promoter are provided, as are host cells comprising such expression vectors, and pharmaceutical compositions comprising such vectors and host cells with one or more pharmaceutically acceptable excipients.

In one aspect, a protein molecule comprising a human Smad7 protein having leucine at position 216 is provided. In some embodiments, the human Smad7 protein may be truncated at the C-terminal, or truncated at the N-terminal. In some embodiments, the truncated human Smad7 protein may include about 50% of the full-length Smad7 sequence, or may include about 13% of the full-length Smad7 sequence. In some embodiments, the human Smad7 protein may comprise or consist of amino acids 203-426 of the human Smad7 protein. In some embodiments, the protein molecule may have one or more biological activities selected from the group consisting of reducing or eliminating phosphorylation of Smad2, reducing or eliminating nuclear translocation of the NF-κB p50 subunit, increasing cell proliferation, reducing apoptosis, reducing radiation-induced DNA damage, reducing inflammation, reducing angiogenesis, promoting healing in oral mucositis, promoting wound healing, and treating auto-immune disease. In some embodiments, any of the foregoing may further comprise a protein transduction domain, such as Tat. In some embodiments, any of the foregoing may also further comprise one or more of an epitope tag or a purification tag, such as V5, glutathione-S-transferase or 6-histidine (SEQ ID NO: 56). In some embodiments, a pharmaceutical composition comprising any of the foregoing, a protein molecule, and one or more pharmaceutically acceptable excipients is provided.

In another aspect, a method is provided for treating or preventing an inflammatory condition in a subject comprising providing to the subject a therapeutically effective amount of the pharmaceutical composition described above. In some embodiments, the inflammatory condition may be one or more of a chronic wound, acute wound, mucositis, skin inflammation, psoriasis, or an autoimmune disease. In some embodiments, the composition may reduce inflammation through inhibition of TGF-β and NF-κB signaling. The method may include treating or preventing injury-associated fibrotic diseases including burn-induced (chemical included) skin and soft tissue scarring and contraction, radiation-induced skin and organ scarring, post cancer therapeutic radiation treatment, and keloid (skin). In some embodiments, radiation-induced skin and organ scarring may include radiodermatitis and radiation-induced lung disease (RILD). In some embodiments, injury-asociated fibrotic diseases may include acute fibrosis, contraction of collagen-including tissue, and fibrotic lung diseases including IPF. In some embodiments, the chronic wounds may include one or more of diabetic ulcers, pressure ulcers, venous ulcers, or oral ulcers, the acute wounds may include one or more of trauma-induced wounds, surgical wounds, or scarring, the mucositis may include one or more of radiation-induced mucositis or chemotherapy-induced mucositis and the mucositis may include one or more of oral mucositis or gut mucositis.

In another aspect, a method is provided for preventing or treating a disease or disorder in a subject comprising one or more of increasing one or more of cell proliferation or cell migration, or preventing one or more of apoptosis or DNA damage in the subject comprising providing to the subject a therapeutically effective amount of the pharmaceutical composition as described above, wherein one or more of increasing one or more of cell proliferation or cell migration, or preventing one or more of apoptosis or DNA damage is useful in preventing or treating the disease or disorder. In some embodiments, the disease or disorder may include one or more of chronic wounds, acute wounds, mucositis, skin inflammation, psoriasis, or an autoimmune disease. In some embodiments, the chronic wounds may include one or more of diabetic ulcers, pressure ulcers, venous ulcers, or oral ulcers, the acute wounds may include one or more of trauma-induced wounds, surgical wounds, or scarring, the mucositis may include one or more of radiation-induced mucositis or chemotherapy-induced mucositis and the mucositis may include one or more of oral mucositis or gut mucositis. The method may include treating or preventing injury-associated fibrotic diseases including burn-induced (chemical included) skin and soft tissue scarring and contraction, radiation-induced skin and organ scarring, post cancer therapeutic radiation treatment, and keloid (skin). In some embodiments, radiation-induced skin and organ scarring may include radiodermatitis and radiation-induced lung disease (RILD). In some embodiments, injury-asociated fibrotic diseases may include acute fibrosis, contraction of collagen-including tissue, and fibrotic lung diseases including IPF.

It is contemplated that any method or composition described herein can be implemented with respect to any other method or composition described herein.

The use of the word “a” or “an” when used in conjunction with the term “comprising” in the claims and/or the specification may mean “one,” but it is also consistent with the meaning of “one or more,” “at least one,” and “one or more than one.” The word “about” means plus or minus 5% of the stated number.

Other objects, features and advantages of the present technology will become apparent from the following detailed description. It should be understood, however, that the detailed description and the specific examples, while indicating specific embodiments of the present technology, are given by way of illustration only, since various changes and modifications within the spirit and scope of the present technology will become apparent to those skilled in the art from this detailed description.

This disclosure includes the following sequences (peptides are displayed using the single-letter amino acid abbreviations). This table provides a partial list of sequences usefule in the technology of this disclosure:

| | |
|---|---|
| Tat-Smad7 (203- | ggatccggcc gtaaaaaacg ccgtcaacgc cgccgtgaac tggagagccc |
| 426 aa)-HA | gccgccgccg tattctcgtt acccgatgga tttcctgaaa ccgactgcag |
| Notes: 1-6: BamHI; | attgcccgga cgcagtcccg tcatcggctg agaccggcgg caccaactat |
| 7-36: Tat; 37-708: | ctggcaccgg gcggtctgag tgattcccag ctgctgctgg aaccgggcga |
| bacterial codon | ccgttcacat tggtgtgtgg ttgcctattg ggaagagaaa acgcgtgtcg |
| optimized human | gtcgcctgta ctgcgtacag gaaccgtcgc tggatatctt ttatgacctg |
| Smad7 203-426; | ccgcagggca atggtttctg tctgggccaa ctgaactcag ataataaatc |
| 709-735: HA: 736- | gcagctggtg caaaaagttc gctcaaaaat tggctgcggt atccagctga |
| 738: stop; 739: SalI; | cccgtgaagt tgacggtgtc tgggtatata accgcagctc ttacccgatt |
| SEQ ID NO: 57 | tttatcaaaa gtgccaccct ggataatccg gactcccgta cgctgctggt |
| | ccacaaagta tttccgggct tctcaatcaa agcgttcgat tacgagaaag |
| | cctactcgct gcagcgcccg aacgaccatg aattcatgca gcaaccgtgg |
| | acgggtttta ctgtgcagat ctctttcgtt aaaggctggg gtcaatgcta |
| | cacccgtcag tttatctcgt cctgtccgtg ctggctggaa gtgattttca |
| | atagccgcta cccatacgac gtcccagact acgcttaggt cgac |
| Translation of 57 | gsgrkkrrqr rrelespppp ysrypmdflk ptadcpdavp ssaetggtny |
| Notes: 3-12: Tat; | lapgglsdsq lllepgdrsh wcvvayweek trvgrlycvq epsldifydl |
| 13-236: hSmac17 | pqgngfclgq lnsdnksqlv qkvrskigcg iqltrevdgv wvynrssypi |
| 203-426 aa; 237- | fiksatldnp dsrtllvhkv fpgfsikafd yekayslqrp ndhefmqqpw |
| 245: HA | tgftvqisfv kgwgqcytrq fisscpcwle vifnsrypyd vpdya |
| SEQ ID NO: 58 | |
| Tat-Smad7 (203- | ggatccggcc gtaaaaaacg ccgtcaacgc cgccgtgaac tggagagccc |
| 426 aa)-HA | gccgccgccg tattctcgtt acccgctgga tttcctgaaa ccgactgcag |
| Note: 76: A to C | attgcccgga cgcagtcccg tcatcggctg agaccggcgg caccaactat |
| mutation, changed | ctggcaccgg gcggtctgag tgattcccag ctgctgctgg aaccgggcga |
| M to L (hSmad7 | ccgttcacat tggtgtgtgg ttgcctattg ggaagagaaa acgcgtgtcg |
| codon 216): 1-6: | gtcgcctgta ctgcgtacag gaaccgtcgc tggatatctt ttatgacctg |
| BamHI; 7-36: Tat; | ccgcagggca atggtttctg tctgggccaa ctgaactcag ataataaatc |
| 37-708: bacterial | gcagctggtg caaaaagttc gctcaaaaat tggctgcggt atccagctga |
| codon optimized | cccgtgaagt tgacggtgtc tgggtatata accgcagctc ttacccgatt |
| human Smad7 203- | tttatcaaaa gtgccaccct ggataatccg gactcccgta cgctgctggt |
| 426; 709-735: HA: | ccacaaagta tttccgggct tctcaatcaa agcgttcgat tacgagaaag |
| 736-738: stop; 739: | cctactcgct gcagcgcccg aacgaccatg aattcatgca gcaaccgtgg |
| SalI | acgggtttta ctgtgcagat ctctttcgtt aaaggctggg gtcaatgcta |
| SEQ ID NO: 59 | cacccgtcag tttatctcgt cctgtccgtg ctggctggaa gtgattttca |
| | atagccgcta cccatacgac gtcccagact acgcttaggt cgac |
| Translation of 59 | gsgrkkrrqr rrelespppp ysrypldflk ptadcpdavp ssaetggtny |
| Notes: hSmad7 | lapgglsdsq lllepgdrsh wcvvayweek trvgrlycvq epsldifydl |
| codon 216 changed | pqgngfclgq lnsdnksqlv qkvrskigcg iqltrevdgv wvynrssypi |
| from M to L; 3-12: | fiksatldnp dsrtllvhkv fpgfsikafd yekayslqrp ndhefmqqpw |
| Tat; 13-236: | tgftvqisfv kgwgqcytrq fisscpcwle vifnsrypyd vpdya |
| hSmad7 203-426 aa; | |
| 237-245: HA | |
| SEQ ID NO: 60 | |
| Tat-Smad7 (203- | ggatccggcc gtaaaaaacg ccgtcaacgc cgccgtgaac tggagagccc |
| 426 aa). | gccgccgccg tattctcgtt acccgatgga tttcctgaaa ccgactgcag |
| Notes: 711 is | attgcccgga cgcagtcccg tcatcggctg agaccggcgg caccaactat |
| mutated from c to g | ctggcaccgg gcggtctgag tgattcccag ctgctgctgg aaccgggcga |
| to create a tag stop | ccgttcacat tggtgtgtgg ttgcctattg ggaagagaaa acgcgtgtcg |
| codon to remove | gtcgcctgta ctgcgtacag gaaccgtcgc tggatatctt ttatgacctg |
| HA. | ccgcagggca atggtttctg tctgggccaa ctgaactcag ataataaatc |
| SEQ ID NO: 61 | gcagctggtg caaaaagttc gctcaaaaat tggctgcggt atccagctga |
| | cccgtgaagt tgacggtgtc tgggtatata accgcagctc ttacccgatt |
| | tttatcaaaa gtgccaccct ggataatccg gactcccgta cgctgctggt |
| | ccacaaagta tttccgggct tctcaatcaa agcgttcgat tacgagaaag |
| | cctactcgct gcagcgcccg aacgaccatg aattcatgca gcaaccgtgg |
| | acgggtttta ctgtgcagat ctctttcgtt aaaggctggg gtcaatgcta |
| | cacccgtcag tttatctcgt cctgtccgtg ctggctggaa gtgattttca |
| | atagccgcta gccatacgac gtcccagact acgcttaggt cgac |
| Translation of 61 | gsgrkkrrqr rrelespppp ysrypmdflk ptadcpdavp ssaetggtny |
| Notes: 3-12: Tat; | lapgglsdsq lllepgdrsh wcvvayweek trvgrlycvq epsldifydl |
| 13-236: hSmad7 | pqgngfclgq lnsdnksqlv qkvrskigcg iqltrevdgv wvynrssypi |
| 203-426 aa | fiksatldnp dsrtllvhkv fpgfsikafd yekayslqrp ndhefmqqpw |
| SEQ ID NO: 62 | tgftvqisfv kgwgqcytrq fisscpcwle vifnsr |
| Tat-Smad7 (203- | ggatccggcc gtaaaaaacg ccgtcaacgc cgccgtgaac tggagagccc |
| 426 aa). 76: A to C | gccgccgccg tattctcgtt acccgctgga tttcctgaaa ccgactgcag |
| mutation, changes | attgcccgga cgcagtcccg tcatcggctg agaccggcgg caccaactat |
| M to L (hSmad7 | ctggcaccgg gcggtctgag tgattcccag ctgctgctgg aaccgggcga |
| codon 216) | ccgttcacat tggtgtgtgg ttgcctattg ggaagagaaa acgcgtgtcg |
| SEQ ID NO: 63 | gtcgcctgta ctgcgtacag gaaccgtcgc tggatatctt ttatgacctg |
| | ccgcagggca atggtttctg tctgggccaa ctgaactcag ataataaatc |
| | gcagctggtg caaaaagttc gctcaaaaat tggctgcggt atccagctga |
| | cccgtgaagt tgacggtgtc tgggtatata accgcagctc ttacccgatt |
| | tttatcaaaa gtgccaccct ggataatccg gactcccgta cgctgctggt |
| | ccacaaagta tttccgggct tctcaatcaa agcgttcgat tacgagaaag |
| | cctactcgct gcagcgcccg aacgaccatg aattcatgca gcaaccgtgg |
| | acgggtttta ctgtgcagat ctctttcgtt aaaggctggg gtcaatgcta |
| | cacccgtcag tttatctcgt cctgtccgtg ctggctggaa gtgattttca |
| | atagccgcta gccatacgac gtcccagact acgcttaggt cgac |
| Translation of 63 | gsgrkkrrqr rrelespppp ysrypldflk ptadcpdavp ssaetggtny |
| (hSnmd7 codon 216 | lapgglsdsq lllepgdrsh wcvvayweek trvgrlycvq epsldifydl |
| changed from M to | pqgngfclgq lnsdnksqlv qkvrskigcg iqltrevdgv wvynrssypi |
| L): 3-12: Tat; 13- | fiksatldnp dsrtllvhkv fpgfsikafd yekayslqrp ndhefmqqpw |
| 236: h5mad7 203- | tgftvqisfv kgwgqcytrq fisscpcwle vifnsr |
| 426 aa | |
| SEQ ID NO: 64 | |
| Tat-Smad7 (203- | ggatccggcc gcaagaaacg ccgccagcgc cgccgcgaac tagagtctcc |
| 426)-HA, human | cccccctcct tactccagat acccgatgga ttttctcaaa ccaactgcag |
| codons: human | actgtccaga tgctgtgcct tcctccgctg aaacaggggg aacgaattat |
| Smad7 codon | ctggcccctg gggggctttc agattcccaa cttcttctgg agcctgggga |
| 7-36: Tat; 37-708: | tcggtcacac tggtgcgtgg tggcatactg ggaggagaag acgagagtgg |
| 203-426 aa human | ggaggctcta ctgtgtccag gagccctctc tggatatctt ctatgatcta |
| Smad7 | cctcagggga atggcttttg cctcggacag ctcaattcgg acaacaagag |
| 709: EcoRI; 710- | tcagctggtg cagaaggtgc ggagcaaaat cggctgcggc atccagctga |
| 741: HA tag | cgcgggaggt ggatggtgtg tgggtgtaca accgcagcag ttaccccatc |
| SEQ ID NO: 65 | ttcatcaagt ccgccacact ggacaacccg gactccagga cgctgttggt |
| | acacaaggtg ttccccggtt tctccatcaa ggctttcgac tacgagaagg |
| | cgtacagcct gcagcggccc aatgaccacg agtttatgca gcagccgtgg |
| | acgggcttta ccgtgcagat cagctttgtg aagggctggg gtcagtgcta |
| | cacccgccag ttcatcagca gctgcccgtg ctggctagag gtcatcttca |
| | acagccggga attctaccca tacgacgtcc cagactacgc ttaggtcgac |
| Translation of 65 | gsgrkkrrqr rrelespppp ysrypmdflk ptadcpdavp ssaetggtny |
| Notes: 3-12: Tat; | lapgglsdsq lllepgdrsh wcvvayweek trvgrlycvq epsldifydl |
| 13-236: hSmad7 | pqgngfclgq lnsdnksqlv qkvrskigcg iqltrevdgv wvynrssypi |
| 203-426 aa; 237- | fiksatldnp dsrtllvhkv fpgfsikafd yekayslqrp ndhefmqqpw |
| 245: HA | tgftvqisfv kgwgqcytrq fisscpcwle vifnsrypyd vpdya |
| SEQ ID NO: 66 | |
| Tat-Smad7 (203- | ggatccggcc gcaagaaacg ccgccagcgc cgccgcgaac tagagtctcc |
| 426)-HA. human | cccccctcct tactccagat acccgctgga ttttctcaaa ccaactgcag |
| codons:. 76: A to C | actgtccaga tgctgtgcct tcctccgctg aaacaggggg aacgaattat |
| mutation, changed | ctggcccctg gggggctttc agattcccaa cttcttctgg agcctgggga |
| M to L (hSmad7 | tcggtcacac tggtgcgtgg tggcatactg ggaggagaag acgagagtgg |
| codon 216) | ggaggctcta ctgtgtccag gagccctctc tggatatctt ctatgatcta |
| SEQ ID NO: 67 | cctcagggga atggcttttg cctcggacag ctcaattcgg acaacaagag |
| | tcagctggtg cagaaggtgc ggagcaaaat cggctgcggc atccagctga |
| | cgcgggaggt ggatggtgtg tgggtgtaca accgcagcag ttaccccatc |
| | ttcatcaagt ccgccacact ggacaacccg gactccagga cgctgttggt |
| | acacaaggtg ttccccggtt tctccatcaa ggctttcgac tacgagaagg |
| | cgtacagcct gcagcggccc aatgaccacg agtttatgca gcagccgtgg |
| | acgggcttta ccgtgcagat cagctttgtg aagggctggg gtcagtgcta |
| | cacccgccag ttcatcagca gctgcccgtg ctggctagag gtcatcttca |
| | acagccggga attctaccca tacgacgtcc cagactacgc ttaggtcgac |
| Translation of 67 | gsgrkkrrqr rrelespppp ysrypldflk ptadcpdavp ssaetggtny |
| hSmad7 codon 216 | lapgglsdsq lllepgdrsh wcvvayweek trvgrlycvq epsldifydl |
| changed from M to | pqgngfclgq lnsdnksqlv qkvrskigcg iqltrevdgv wvynrssypi |
| L; Notes: 3-12: Tat; | fiksatldnp dsrtllvhkv fpgfsikafd yekayslqrp ndhefmqqpw |
| 13-236: hSmad7 | tgftvqisfv kgwgqcytrq fisscpcwle vifnsrypyd vpdya |
| 203-426 aa; 237- | |
| 245: HA | |
| SEQ ID NO: 68 | |
| Tat-Smad7 (203- | ggatccggcc gcaagaaacg ccgccagcgc cgccgcgaac tagagtctcc |
| 426), human | cccccctcct tactccagat acccgatgga ttttctcaaa ccaactgcag |
| codons: | actgtccaga tgctgtgcct tcctccgctg aaacaggggg aacgaattat |
| 709: g to t mutation | ctggcccctg gggggctttc agattcccaa cttcttctgg agcctgggga |
| to delete HA | tcggtcacac tggtgcgtgg tggcatactg ggaggagaag acgagagtgg |
| SEQ ID NO: 69 | ggaggctcta ctgtgtccag gagccctctc tggatatctt ctatgatcta |
| | cctcagggga atggcttttg cctcggacag ctcaattcgg acaacaagag |
| | tcagctggtg cagaaggtgc ggagcaaaat cggctgcggc atccagctga |
| | cgcgggaggt ggatggtgtg tgggtgtaca accgcagcag ttaccccatc |
| | ttcatcaagt ccgccacact ggacaacccg gactccagga cgctgttggt |
| | acacaaggtg ttccccggtt tctccatcaa ggctttcgac tacgagaagg |
| | cgtacagcct gcagcggccc aatgaccacg agtttatgca gcagccgtgg |
| | acgggcttta ccgtgcagat cagctttgtg aagggctggg gtcagtgcta |
| | cacccgccag ttcatcagca gctgcccgtg ctggctagag gtcatcttca |
| | acagccggta attctaccca tacgacgtcc cagactacgc ttaggtcgac |
| Translation of 69 | gsgrkkrrqr rrelespppp ysrypmdflk ptadcpdavp ssaetggtny |
| 3-12: Tat 13-236: | lapgglsdsq lllepgdrsh wcvvayweek trvgrlycvq epsldifydl |
| hSmad7 203-426 aa | pqgngfclgq lnsdnksqlv qkvrskigcg iqltrevdgv wvynrssypi |
| SEQ ID NO: 70 | fiksatldnp dsrtllvhkv fpgfsikafd yekayslqrp ndhefmqqpw |
| | tgftvqisfv kgwgqcytrq fisscpcwle vifnsr |
| Tat-smad7 (203- | ggatccggcc gcaagaaacg ccgccagcgc cgccgcgaac tagagtctcc |
| 426) human | cccccctcct tactccagat acccgctgga ttttctcaaa ccaactgcag |
| codons: 76: A to C | actgtccaga tgctgtgcct tcctccgctg aaacaggggg aacgaattat |
| mutation, changed | ctggcccctg gggggctttc agattcccaa cttcttctgg agcctgggga |
| M to L (hSmad7 | tcggtcacac tggtgcgtgg tggcatactg ggaggagaag acgagagtgg |
| codon 216) | ggaggctcta ctgtgtccag gagccctctc tggatatctt ctatgatcta |
| SEQ ID NO: 71 | cctcagggga atggcttttg cctcggacag ctcaattcgg acaacaagag |
| | tcagctggtg cagaaggtgc ggagcaaaat cggctgcggc atccagctga |
| | cgcgggaggt ggatggtgtg tgggtgtaca accgcagcag ttaccccatc |
| | ttcatcaagt ccgccacact ggacaacccg gactccagga cgctgttggt |
| | acacaaggtg ttccccggtt tctccatcaa ggctttcgac tacgagaagg |
| | cgtacagcct gcagcggccc aatgaccacg agtttatgca gcagccgtgg |
| | acgggcttta ccgtgcagat cagctttgtg aagggctggg gtcagtgcta |
| | cacccgccag ttcatcagca gctgcccgtg ctggctagag gtcatcttca |
| | acagccggta attctaccca tacgacgtcc cagactacgc ttaggtcgac |
| Translation of 71 | gsgrkkrrqr rrelespppp ysrypldflk ptadcpdavp ssaetggtny |
| hSmad7 codon 216 | lapgglsdsq lllepgdrsh wcvvayweek trvgrlycvq epsldifydl |
| changed from M to | pqgngfclgq lnsdnksqlv qkvrskigcg iqltrevdgv wvynrssypi |
| L | fiksatldnp dsrtllvhkv fpgfsikafd yekayslqrp ndhefmqqpw |
| Notes: 3-12: Tat; | tgftvqisfv kgwgqcytrq fisscpcwle vifnsr |
| 13-236: hSmad7 | |
| 203-426 aa | |
| SEQ ID NO: 72 | |
| Tat-Smad7 (203- | ttcccctcta gaaataattt tgtttaactt taagaaggaa ttcaggagcc |
| 426)-V5-His | cttcaccatg cgtaaaaaac gccgtcaacg ccgccgtgaa ctggagagcc |
| Notes: 58-759: Tat- | cgccgccgcc gtattctcgt tacccgatgg atttcctgaa accgactgca |
| hSmad7 203-426 aa; | gattgcccgg acgcagtccc gtcatcggct gagaccggcg gcaccaacta |
| V5: 787-828. His | tctggcaccg ggcggtctga gtgattccca gctgctgctg gaaccgggcg |
| tag: 838-855. Stop: | accgttcaca ttggtgtgtg gttgcctatt gggaagagaa aacgcgtgtc |
| 856 | ggtcgcctgt actgcgtaca ggaaccgtcg ctggatatct tttatgacct |
| SEQ ID NO: 73 | gccgcagggc aatggtttct gtctgggcca actgaactca gataataaat |
| | cgcagctggt gcaaaaagtt cgctcaaaaa ttggctgcgg tatccagctg |
| | acccgtgaag ttgacggtgt ctgggtatat aaccgcagct cttacccgat |
| | ttttatcaaa agtgccaccc tggataatcc ggactcccgt acgctgctgg |
| | tccacaaagt atttccgggc ttctcaatca aagcgttcga ttacgagaaa |
| | gcctactcgc tgcagcgccc gaacgaccat gaattcatgc agcaaccgtg |
| | gacgggtttt actgtgcaga tctctttcgt taaaggctgg ggtcaatgct |
| | acacccgtca gtttatctcg tcctgtccgt gctggctgga agtgattttc |
| | aatagccgca agggcgagct caattcgaag cttgaaggta agcctatccc |
| | taaccctctc ctcggtctcg attctacgcg taccggtcat catcaccatc |
| | accattgagt ttgatccggc tgctaacaaa gcccgaaagg a |
| Translation of 73 | mrkkrrqrrr elesppppys rypmdflkpt adcpdavpss aetggtnyla |
| Notes: 1-10: Tat; | pgglsdsqll lepgdrshwc vvayweektr vgrlycvqep sldifydlpq |
| 11-234: hSmad7 | gngfclgqln sdnksqlvqk vrskigcgiq ltrevdgvwv ynrssypifi |
| (203-426); 244-257: | ksatldnpds rtllvhkvfp gfsikafdye kayslqrpnd hefmqqpwtg |
| V5; 261-266: His | ftvqisfvkg wgqcytrqfi sscpcwlevi fnsrkgelns klegkpipnp |
| tag | llgldstrtg hhhhhh |
| SEQ ID NO: 74 | |
| Mutant Tat-Smad7 | ttcccctcta gaaataattt tgtttaactt taagaaggaa ttcaggagcc |
| (203-426)-V5-His, | cttcaccatg cgtaaaaaac gccgtcaacg ccgccgtgaa ctggagagcc |
| 127 from A to C | cgccgccgcc gtattctcgt tacccgctgg atttcctgaa accgactgca |
| mutation, changing | gattgcccgg acgcagtccc gtcatcggct gagaccggcg gcaccaacta |
| M to L | tctggcaccg ggcggtctga gtgattccca gctgctgctg gaaccgggcg |
| SEQ ID NO: 75 | accgttcaca ttggtgtgtg gttgcctatt gggaagagaa aacgcgtgtc |
| | ggtcgcctgt actgcgtaca ggaaccgtcg ctggatatct tttatgacct |
| | gccgcagggc aatggtttct gtctgggcca actgaactca gataataaat |
| | cgcagctggt gcaaaaagtt cgctcaaaaa ttggctgcgg tatccagctg |
| | acccgtgaag ttgacggtgt ctgggtatat aaccgcagct cttacccgat |
| | ttttatcaaa agtgccaccc tggataatcc ggactcccgt acgctgctgg |
| | tccacaaagt atttccgggc ttctcaatca aagcgttcga ttacgagaaa |
| | gcctactcgc tgcagcgccc gaacgaccat gaattcatgc agcaaccgtg |
| | gacgggtttt actgtgcaga tctctttcgt taaaggctgg ggtcaatgct |
| | acacccgtca gtttatctcg tcctgtccgt gctggctgga agtgattttc |
| | aatagccgca agggcgagct caattcgaag cttgaaggta agcctatccc |
| | taaccctctc ctcggtctcg attctacgcg taccggtcat catcaccatc |
| | accattgagt ttgatccggc tgctaacaaa gcccgaaagg a |
| Translation of 75 | mrkkrrqrrr elesppppys rypldflkpt adcpdavpss aetggtnyla |
| hSmad7 codon 216 | pgglsdsqll lepgdrshwc vvayweektr vgrlycvqep sldifydlpq |
| changed from M to | gngfclgqln sdnksqlvqk vrskigcgiq ltrevdgvwv ynrssypifi |
| L | ksatldnpds rtllvhkvfp gfsikafdye kayslqrpnd hefmqqpwtg |
| SEQ ID NO: 76 | ftvqisfvkg wgqcytrqfi sscpcwlevi fnsrkgelns klegkpipnp |
| | llgldstrtg hhhhhh |
| Tat-PY | GRKKRRQRRRELESPPPPYSRYPMD |
| SEQ ID NO: 77 | |
| PY | ELESPPPPYSRYPMD |
| SEQ ID NO: 78 | |
| Pep1-Smad7(203- | ggatccaaag aaacctggtg ggaaacctgg tggaccgaat ggagccagcc |
| 426)HA nucleotides | gaaaaagaaa cgtaaagtgg aactggagag cccgccgccg ccgtattctc |
| SEQ ID: 79 | gttacccgat ggatttcctg aaaccgactg cagattgccc ggacgcagtc |
| | ccgtcatcgg ctgagaccgg cggcaccaac tatctggcac cgggcggtct |
| | gagtgattcc cagctgctgc tggaaccggg cgaccgttca cattggtgtg |
| | tggttgccta ttgggaagag aaaacgcgtg tcggtcgcct gtactgcgta |
| | caggaaccgt cgctggatat cttttatgac ctgccgcagg gcaatggttt |
| | ctgtctgggc caactgaact cagataataa atcgcagctg gtgcaaaaag |
| | ttcgctcaaa aattggctgc ggtatccagc tgacccgtga agttgacggt |
| | gtctgggtat ataaccgcag ctcttacccg atttttatca aaagtgccac |
| | cctggataat ccggactccc gtacgctgct ggtccacaaa gtatttccgg |
| | gcttctcaat caaagcgttc gattacgaga aagcctactc gctgcagcgc |
| | ccgaacgacc atgaattcat gcagcaaccg tggacgggtt ttactgtgca |
| | gatctctttc gttaaaggct ggggtcaatg ctacacccgt cagtttatct |
| | cgtcctgtcc gtgctggctg gaagtgattt tcaatagccg ctacccatac |
| | gacgtcccag actacgctta ggtcgac |
| Translation of 79 | gsketwwetw wtewsqpkkk rkvelesppp pysrypmdfl kptadcpdav |
| Pep1-Smad7(203- | pssaetggtn ylapgglsds qlllepgdrs hwcvvaywee ktrvgrlycv |
| 426)HA aa | qepsldifyd lpqgngfclg qlnsdnksql vqkvrskigc giqltrevdg |
| SEQ ID: 80 | vwvynrssyp ifiksatldn pdsrtllvhk vfpgfsikaf dyekayslqr |
| | pndhefmqqp wtgftvqisf vkgwgqcytr qfisscpcwl evifnsrypy |
| | dvpdya |
| Pep1Smad7(203- | ggatccaaag aaacctggtg ggaaacctgg tggaccgaat ggagccagcc |
| 426)nucleotides, no | gaaaaagaaa cgtaaagtgg aactggagag cccgccgccg ccgtattctc |
| HA | gttacccgat ggatttcctg aaaccgactg cagattgccc ggacgcagtc |
| SEQ ID: 81 | ccgtcatcgg ctgagaccgg cggcaccaac tatctggcac cgggcggtct |
| | gagtgattcc cagctgctgc tggaaccggg cgaccgttca cattggtgtg |
| | tggttgccta ttgggaagag aaaacgcgtg tcggtcgcct gtactgcgta |
| | caggaaccgt cgctggatat cttttatgac ctgccgcagg gcaatggttt |
| | ctgtctgggc caactgaact cagataataa atcgcagctg gtgcaaaaag |
| | ttcgctcaaa aattggctgc ggtatccagc tgacccgtga agttgacggt |
| | gtctgggtat ataaccgcag ctcttacccg atttttatca aaagtgccac |
| | cctggataat ccggactccc gtacgctgct ggtccacaaa gtatttccgg |
| | gcttctcaat caaagcgttc gattacgaga aagcctactc gctgcagcgc |
| | ccgaacgacc atgaattcat gcagcaaccg tggacgggtt ttactgtgca |
| | gatctctttc gttaaaggct ggggtcaatg ctacacccgt cagtttatct |
| | cgtcctgtcc gtgctggctg gaagtgattt tcaatagccg ctagccatac |
| | gacgtcccag actacgctta ggtcgac |
| Translation of 81 | gsketwwetw wtewsqpkkk rkvelesppp pysrypmdfl kptadcpdav |
| Pep1Smad7(203- | pssaetggtn ylapgglsds qlllepgdrs hwcvvaywee ktrvgrlycv |
| 426) aa, no HA | qepsldifyd lpqgngfclg qlnsdnksql vqkvrskigc giqltrevdg |
| SEQ ID:82 | vwvynrssyp ifiksatldn pdsrtllvhk vfpgfsikaf dyekayslqr |
| | pndhefmqqp wtgftvqisf vkgwgqcytr qfisscpcwl evifnsr |
| Pep1Smad7V5 | ggatccaaag aaacctggtg ggaaacctgg tggaccgaat ggagccagcc |
| nucleotides | gaaaaagaaa cgtaaagtgg gtttccgtac gaaacgctcg gccctggtcc |
| SEQ ID: 83 | gtcgcctgtg gcgctcccgt gctccgggtg gtgaagatga agaagaaggt |
| | gctggcggcg gtggcggtgg cggtgaactg cgtggcgagg gtgcaaccga |
| | tagtcgtgca cacggtgcag gcggtggcgg tccgggtcgt gctggttgct |
| | gtctgggtaa agctgtgcgc ggcgcgaaag gtcatcacca tccgcacccg |
| | ccggcagcag gtgcaggtgc agctggcggt gcggaagccg atctgaaagc |
| | cctgacccat agtgtcctga aaaaactgaa agaacgtcag ctggagctgc |
| | tgctgcaagc agtagaatcc cgtggcggta cccgtacggc ttgtctgctg |
| | ctgccgggtc gtctggattg ccgtctgggt ccgggtgcac cggctggtgc |
| | gcagccggca caaccgccga gctcttacag cctgccgctg ctgctgtgta |
| | aagtgtttcg ttggccggac ctgcgccaca gttccgaagt taaacgcctg |
| | tgctgttgcg agagctatgg caaaattaac ccggaactgg tttgttgcaa |
| | tccgcaccat ctgtctcgtc tgtgtgaact ggagagcccg ccgccgccgt |
| | attctcgtta cccgatggat ttcctgaaac cgactgcaga ttgcccggac |
| | gcagtcccgt catcggctga gaccggcggc accaactatc tggcaccggg |
| | cggtctgagt gattcccagc tgctgctgga accgggcgac cgttcacatt |
| | ggtgtgtggt tgcctattgg gaagagaaaa cgcgtgtcgg tcgcctgtac |
| | tgcgtacagg aaccgtcgct ggatatcttt tatgacctgc cgcagggcaa |
| | tggtttctgt ctgggccaac tgaactcaga taataaatcg cagctggtgc |
| | aaaaagttcg ctcaaaaatt ggctgcggta tccagctgac ccgtgaagtt |
| | gacggtgtct gggtatataa ccgcagctct tacccgattt ttatcaaaag |
| | tgccaccctg gataatccgg actcccgtac gctgctggtc cacaaagtat |
| | ttccgggctt ctcaatcaaa gcgttcgatt acgagaaagc ctactcgctg |
| | cagcgcccga acgaccatga attcatgcag caaccgtgga cgggttttac |
| | tgtgcagatc tctttcgtta aaggctgggg tcaatgctac acccgtcagt |
| | ttatctcgtc ctgtccgtgc tggctggaag tgattttcaa tagccgcaag |
| | ggcgagctca attcgaagct tgaaggtaag cctatcccta accctctcct |
| | cggtctcgat tctacgtgag tcgac |
| Translation of 83 | gsketwwetw wtewsqpkkk rkvgfrtkrs alvrrlwrsr apggedeeeg |
| Pep1Smad7V5 aa | agggggggel rgegatdsra hgaggggpgr agcclgkavr gakghhhphp |
| SEQ ID: 84 | paagagaagg aeadlkalth svlkklkerq lelllqaves rggtrtacll |
| | lpgrldcrlg pgapagaqpa qppssyslpl llckvfrwpd lrhssevkrl |
| | cccesygkin pelvccnphh lsrlcelesp pppysrypmd flkptadcpd |
| | avpssaetgg tnylapggls dsqlllepgd rshwcvvayw eektrvgrly |
| | cvqepsldif ydlpqgngfc lgqlnsdnks qlvqkvrski gcgiqltrev |
| | dgvwvynrss ypifiksatl dnpdsrtllv hkvfpgfsik afdyekaysl |
| | qrpndhefmq qpwtgftvqi sfvkgwgqcy trqfisscpc wlevifnsrk |
| | gelnsklegk pipnpllgld st |
| Pep1Smad7, no V5 | ggatccaaag aaacctggtg ggaaacctgg tggaccgaat ggagccagcc |
| nucleotides | gaaaaagaaa cgtaaagtgg gtttccgtac gaaacgctcg gccctggtcc |
| SEQ ID: 85 | gtcgcctgtg gcgctcccgt gctccgggtg gtgaagatga agaagaaggt |
| | gctggcggcg gtggcggtgg cggtgaactg cgtggcgagg gtgcaaccga |
| | tagtcgtgca cacggtgcag gcggtggcgg tccgggtcgt gctggttgct |
| | gtctgggtaa agctgtgcgc ggcgcgaaag gtcatcacca tccgcacccg |
| | ccggcagcag gtgcaggtgc agctggcggt gcggaagccg atctgaaagc |
| | cctgacccat agtgtcctga aaaaactgaa agaacgtcag ctggagctgc |
| | tgctgcaagc agtagaatcc cgtggcggta cccgtacggc ttgtctgctg |
| | ctgccgggtc gtctggattg ccgtctgggt ccgggtgcac cggctggtgc |
| | gcagccggca caaccgccga gctcttacag cctgccgctg ctgctgtgta |
| | aagtgtttcg ttggccggac ctgcgccaca gttccgaagt taaacgcctg |
| | tgctgttgcg agagctatgg caaaattaac ccggaactgg tttgttgcaa |
| | tccgcaccat ctgtctcgtc tgtgtgaact ggagagcccg ccgccgccgt |
| | attctcgtta cccgatggat ttcctgaaac cgactgcaga ttgcccggac |
| | gcagtcccgt catcggctga gaccggcggc accaactatc tggcaccggg |
| | cggtctgagt gattcccagc tgctgctgga accgggcgac cgttcacatt |
| | ggtgtgtggt tgcctattgg gaagagaaaa cgcgtgtcgg tcgcctgtac |
| | tgcgtacagg aaccgtcgct ggatatcttt tatgacctgc cgcagggcaa |
| | tggtttctgt ctgggccaac tgaactcaga taataaatcg cagctggtgc |
| | aaaaagttcg ctcaaaaatt ggctgcggta tccagctgac ccgtgaagtt |
| | gacggtgtct gggtatataa ccgcagctct tacccgattt ttatcaaaag |
| | tgccaccctg gataatccgg actcccgtac gctgctggtc cacaaagtat |
| | ttccgggctt ctcaatcaaa gcgttcgatt acgagaaagc ctactcgctg |
| | cagcgcccga acgaccatga attcatgcag caaccgtgga cgggttttac |
| | tgtgcagatc tctttcgtta aaggctgggg tcaatgctac acccgtcagt |
| | ttatctcgtc ctgtccgtgc tggctggaag tgattttcaa tagccgctag |
| | gtcgac |
| Translation of 85 | gsketwwetw wtewsqpkkk rkvgfrtkrs alvrrlwrsr apggedeeeg |
| Pep1Smad7 aa | agggggggel rgegatdsra hgaggggpgr agcclgkavr gakghhhphp |
| SEQ ID: 86 | paagagaagg aeadlkalth svlkklkerq lelllqaves rggtrtacll |
| | lpgrldcrlg pgapagaqpa qppssyslpl llckvfrwpd lrhssevkrl |
| | cccesygkin pelvccnphh lsrlcelesp pppysrypmd flkptadcpd |
| | avpssaetgg tnylapggls dsqlllepgd rshwcvvayw eektrvgrly |
| | cvqepsldif ydlpqgngfc lgqlnsdnks qlvqkvrski gcgiqltrev |
| | dgvwvynrss ypifiksatl dnpdsrtllv hkvfpgfsik afdyekaysl |
| | qrpndhefmq qpwtgftvqi sfvkgwgqcy trqfisscpc wlevifnsr |
| Tat-Smad7 | ggatccggcc gtaaaaaacg ccgtcaacgc cgccgtggtt tccgtacgaa |
| nucleotides, no V5 | acgctcggcc ctggtccgtc gcctgtggcg ctcccgtgct ccgggtggtg |
| SEQ ID: 87 | aagatgaaga agaaggtgct ggcggcggtg gcggtggcgg tgaactgcgt |
| | ggcgagggtg caaccgatag tcgtgcacac ggtgcaggcg gtggcggtcc |
| | gggtcgtgct ggttgctgtc tgggtaaagc tgtgcgcggc gcgaaaggtc |
| | atcaccatcc gcacccgccg gcagcaggtg caggtgcagc tggcggtgcg |
| | gaagccgatc tgaaagccct gacccatagt gtcctgaaaa aactgaaaga |
| | acgtcagctg gagctgctgc tgcaagcagt agaatcccgt ggcggtaccc |
| | gtacggcttg tctgctgctg ccgggtcgtc tggattgccg tctgggtccg |
| | ggtgcaccgg ctggtgcgca gccggcacaa ccgccgagct cttacagcct |
| | gccgctgctg ctgtgtaaag tgtttcgttg gccggacctg cgccacagtt |
| | ccgaagttaa acgcctgtgc tgttgcgaga gctatggcaa aattaacccg |
| | gaactggttt gttgcaatcc gcaccatctg tctcgtctgt gtgaactgga |
| | gagcccgccg ccgccgtatt ctcgttaccc gatggatttc ctgaaaccga |
| | ctgcagattg cccggacgca gtcccgtcat cggctgagac cggcggcacc |
| | aactatctgg caccgggcgg tctgagtgat tcccagctgc tgctggaacc |
| | gggcgaccgt tcacattggt gtgtggttgc ctattgggaa gagaaaacgc |
| | gtgtcggtcg cctgtactgc gtacaggaac cgtcgctgga tatcttttat |
| | gacctgccgc agggcaatgg tttctgtctg ggccaactga actcagataa |
| | taaatcgcag ctggtgcaaa aagttcgctc aaaaattggc tgcggtatcc |
| | agctgacccg tgaagttgac ggtgtctggg tatataaccg cagctcttac |
| | ccgattttta tcaaaagtgc caccctggat aatccggact cccgtacgct |
| | gctggtccac aaagtatttc cgggcttctc aatcaaagcg ttcgattacg |
| | agaaagccta ctcgctgcag cgcccgaacg accatgaatt catgcagcaa |
| | ccgtggacgg gttttactgt gcagatctct ttcgttaaag gctggggtca |
| | atgctacacc cgtcagttta tctcgtcctg tccgtgctgg ctggaagtga |
| | ttttcaatag ccgctaggtc gac |
| Translation of 87 | mgrkkrrqrr rgfrtkrsal vrrlwrsrap ggedeeegag ggggggelrg |
| Tat-Smad7 aa, no | egatdsrahg aggggpgrag cclgkavrga kghhhphppa agagaaggae |
| V5 | adlkalthsv lkklkerqle lllqavesrg gtrtaclllp grldcrlgpg |
| SEQ ID: 88 | apagaqpaqp pssyslplll ckvfrwpdlr hssevkrlcc cesygkinpe |
| | lvccnphhls rlcelesppp pysrypmdfl kptadcpdav pssaetggtn |
| | ylapgglsds qlllepgdrs hwcvvaywee ktrvgrlycv qepsldifyd |
| | 1pqgngfclg qlnsdnksql vqkvrskigc giqltrevdg vwvynrssyp |
| | ifiksatldn pdsrtllvhk vfpgfsikaf dyekayslqr pndhefmqqp |
| | wtgftvqisf vkgwgqcytr qfisscpcwl evifnsr |
| Tat-Smad7-V5-His | ttcccctctagaaataattttgtttaactttaagaaggaattcaggagcccttcacc |
| inpET-TOPO | atg cgt aaa aaa cgc cgt caa cgc cgc cgt ggt ttc cgt acg aaa |
| vector | cgc tcg gcc ctg gtc cgt cgc ctg tgg cgc tcc cgt gct ccg ggt |
| SEQ ID: 89 | ggt gaa gat gaa gaa gaa ggt gct ggc ggc ggt ggc ggt ggc ggt |
| | gaa ctg cgt ggc gag ggt gca acc gat agt cgt gca cac ggt gca |
| | ggc ggt ggc ggt ccg ggt cgt gct ggt tgc tgt ctg ggt aaa gct |
| | gtg cgc ggc gcg aaa ggt cat cac cat ccg cac ccg ccg gca gca |
| | ggt gca ggt gca gct ggc ggt gcg gaa gcc gat ctg aaa gcc ctg |
| | acc cat agt gtc ctg aaa aaa ctg aaa gaa cgt cag ctg gag ctg |
| | ctg ctg caa gca gta gaa tcc cgt ggc ggt acc cgt acg gct tgt |
| | ctg ctg ctg ccg ggt cgt ctg gat tgc cgt ctg ggt ccg ggt gca |
| | ccg gct ggt gcg cag ccg gca caa ccg ccg agc tct tac agc ctg |
| | ccg ctg ctg ctg tgt aaa gtg ttt cgt tgg ccg gac ctg cgc cac |
| | agt tcc gaa gtt aaa cgc ctg tgc tgt tgc gag agc tat ggc aaa |
| | att aac ccg gaa ctg gtt tgt tgc aat ccg cac cat ctg tct cgt |
| | ctg tgt gaa ctg gag agc ccg ccg ccg ccg tat tct cgt tac ccg |
| | atg gat ttc ctg aaa ccg act gca gat tgc ccg gac gca gtc ccg |
| | tca tcg gct gag acc ggc ggc acc aac tat ctg gca ccg ggc ggt |
| | ctg agt gat tcc cag ctg ctg ctg gaa ccg ggc gac cgt tca cat |
| | tgg tgt gtg gtt gcc tat tgg gaa gag aaa acg cgt gtc ggt cgc |
| | ctg tac tgc gta cag gaa ccg tcg ctg gat atc ttt tat gac ctg |
| | ccg cag ggc aat ggt ttc tgt ctg ggc caa ctg aac tca gat aat |
| | aaa tcg cag ctg gtg caa aaa gtt cgc tca aaa att ggc tgc ggt |
| | atc cag ctg acc cgt gaa gtt gac ggt gtc tgg gta tat aac cgc |
| | agc tct tac ccg att ttt atc aaa agt gcc acc ctg gat aat ccg |
| | gac tcc cgt acg ctg ctg gtc cac aaa gta ttt ccg ggc ttc tca |
| | atc aaa gcg ttc gat tac gag aaa gcc tac tcg ctg cag cgc ccg |
| | aac gac cat gaa ttc atg cag caa ccg tgg acg ggt ttt act gtg |
| | cag atc tct ttc gtt aaa ggc tgg ggt caa tgc tac acc cgt cag |
| | ttt atc tcg tcc tgt ccg tgc tgg ctg gaa gtg att ttc aat agc |
| | cgc aag ggc gag ctc aat tcg aag ctt gaa ggt aag cct atc cct |
| | aac cct ctc ctc ggt ctc gat tct acg cgt acc ggt cat cat cac |
| | cat cac cat tgagtttgat ccggctgcta acaaagcccg aaagga |
| Ta-human Smad7, | MRKKRRQRRRGFRTKRSALVRRLWRSRAPGGEDEEEGAGGGGGGGELRGEGATDSRAHGA |
| codon optimized | GGGGPGRAGCCLGKAVRGAKGHHHPHPPAAGAGAAGGAEADLKALTHSVLKKLKERQLEL |
| SEQ ID: 90 | LLQAVESRGGTRTACLLLPGRLDCRLGPGAPAGAQPAQPPSSYSLPLLLCKVFRWPDLRH |
| | SSEVKRLCCCESYGKINPELVCCNPHHLSRLCELESPPPPYSRYPMDFLKPTADCPDAVP |
| | SSAETGGTNYLAPGGLSDSQLLLEPGDRSHWCVVAYWEEKTRVGRLYCVQEPSLDIFYDL |
| | PQGNGFCLGQLNSDNKSQLVQKVRSKIGCGIQLTREVDGVWVYNRSSYPIFIKSATLDNP |
| | DSRTLLVHKVFPGFSIKAFDYEKAYSLQRPNDHEFMQQPWTGFTVQISFVKGWGQCYTRQ |
| | FISSCPCWLEVIFNSRKGELNSKLEGKPIPNPLLGLDSTRTGHHHHHH |
| Tat-Smad7-V5 | GSGRKKRRQRRRGFRTKRSALVRRLWRSRAPGGEDEEEGAGGGGGGGELRGEGATDSRAH |
| SEQ ID: 91 | GAGGGGPGRAGCCLGKAVRGAKGHHHPHPPAAGAGAAGGAEADLKALTHSVLKKLKERQL |
| | ELLLQAVESRGGTRTACLLLPGRLDCRLGPGAPAGAQPAQPPSSYSLPLLLCKVFRWPDL |
| | RHSSEVKRLCCCESYGKINPELVCCNPHHLSRLCELESPPPPYSRYPMDFLKPTADCPDA |
| | VPSSAETGGTNYLAPGGLSDSQLLLEPGDRSHWCVVAYWEEKTRVGRLYCVQEPSLDIFY |
| | DLPQGNGFCLGQLNSDNKSQLVQKVRSKIGCGIQLTREVDGVWVYNRSSYPIFIKSATLD |
| | NPDSRTLLVHKVFPGFSIKAFDYEKAYSLQRPNDHEFMQQPWTGFTVQISFVKGWGQCYT |
| | RQFISSCPCWLEVIFNSRKGELNSKLEGKPIPNPLLGLDST |
| Tat-Snmd7 codon | ggatccggcc gtaaaaaacg ccgtcaacgc cgccgtggtt tccgtacgaa |
| opMnized for E. | acgctcggcc ctggtccgtc gcctgtggcg ctcccgtgct ccgggtggtg |
| Coli | aagatgaaga agaaggtgct ggcggcggtg gcggtggcgg tgaactgcgt |
| SEQ ID: 92 | ggcgagggtg caaccgatag tcgtgcacac ggtgcaggcg gtggcggtcc |
| | gggtcgtgct ggttgctgtc tgggtaaagc tgtgcgcggc gcgaaaggtc |
| | atcaccatcc gcacccgccg gcagcaggtg caggtgcagc tggcggtgcg |
| | gaagccgatc tgaaagccct gacccatagt gtcctgaaaa aactgaaaga |
| | acgtcagctg gagctgctgc tgcaagcagt agaatcccgt ggcggtaccc |
| | gtacggcttg tctgctgctg ccgggtcgtc tggattgccg tctgggtccg |
| | ggtgcaccgg ctggtgcgca gccggcacaa ccgccgagct cttacagcct |
| | gccgctgctg ctgtgtaaag tgtttcgttg gccggacctg cgccacagtt |
| | ccgaagttaa acgcctgtgc tgttgcgaga gctatggcaa aattaacccg |
| | gaactggttt gttgcaatcc gcaccatctg tctcgtctgt gtgaactgga |
| | gagcccgccg ccgccgtatt ctcgttaccc gatggatttc ctgaaaccga |
| | ctgcagattg cccggacgca gtcccgtcat cggctgagac cggcggcacc |
| | aactatctgg caccgggcgg tctgagtgat tcccagctgc tgctggaacc |
| | gggcgaccgt tcacattggt gtgtggttgc ctattgggaa gagaaaacgc |
| | gtgtcggtcg cctgtactgc gtacaggaac cgtcgctgga tatcttttat |
| | gacctgccgc agggcaatgg tttctgtctg ggccaactga actcagataa |
| | taaatcgcag ctggtgcaaa aagttcgctc aaaaattggc tgcggtatcc |
| | agctgacccg tgaagttgac ggtgtctggg tatataaccg cagctcttac |
| | ccgattttta tcaaaagtgc caccctggat aatccggact cccgtacgct |
| | gctggtccac aaagtatttc cgggcttctc aatcaaagcg ttcgattacg |
| | agaaagccta ctcgctgcag cgcccgaacg accatgaatt catgcagcaa |
| | ccgtggacgg gttttactgt gcagatctct ttcgttaaag gctggggtca |
| | atgctacacc cgtcagttta tctcgtcctg tccgtgctgg ctggaagtga |
| | ttttcaatag ccgcaagggc gagctcaatt cgaagcttga aggtaagcct |
| | atccctaacc ctctcctcgg tctcgattct acgtgagtcg ac |
| Tat-C-Smad7-V5 | gga tcc ggc cgt aaa aaa cgc cgt caa cgc cgc cgt tca cat tgg |
| Most optimized | tgt gtg gtt gcc tat tgg gaa gag aaa acg cgt gtc ggt cgc ctg |
| includes V5 epitope | tac tgc gta cag gaa ccg tcg ctg gat atc ttt tat gac ctg ccg |
| and pET101-Topo | cag ggc aat ggt ttc tgt ctg ggc caa ctg aac tca gat aat aaa |
| backbone | tcg cag ctg gtg caa aaa gtt cgc tca aaa att ggc tgc ggt atc |
| SEQ ID: 93 | cag ctg acc cgt gaa gtt gac ggt gtc tgg gta tat aac cgc agc |
| | tct tac ccg att ttt atc aaa agt gcc acc ctg gat aat ccg gac |
| | tcc cgt acg ctg ctg gtc cac aaa gta ttt ccg ggc ttc tca atc |
| | aaa gcg ttc gat tac gag aaa gcc tac tcg ctg cag cgc ccg aac |
| | gac cat gaa ttc atg cag caa ccg tgg acg ggt ttt act gtg cag |
| | atc tct ttc gtt aaa ggc tgg ggt caa tgc tac acc cgt cag ttt |
| | atc tcg tcc tgt ccg tgc tgg ctg gaa gtg att ttc aat agc cgc |
| | aag ggc gag ctc aat tcg aag ctt gaa ggt aag cct atc cct aac |
| | cct ctc ctc ggt ctc gat tct acg tgagtcgac |
| Translation of 93 | GSGRKKRRQRRRSHWCVVAYWEEKTRVGRLYCVQEPSLDIFYDLPQGNGFCLGQLNSDNK |
| SEQ ID: 94 | SQLVQKVRSKIGCGIQLTREVDGVWVYNRSSYPIFIKSATLDNPDSRTLLVHKVFPGFSI |
| | KAFDYEKAYSLQRPNDHEFMQQPWTGFTVQISFVKGWGQCYTRQFISSCPCWLEVIFNSR |
| | KGELNSKLEGKPIPNPLLGLDST |
| Tat-Smad7 | gga tcc ggc cgt aaa aaa cgc cgt caa cgc cgc cgt ggt ttc cgt |
| Encodes M216L | acg aaa cgc agc gcc ctg gtc cgt cgc ctg tgg cgc agc cgt gct |
| mutation | ccg ggt ggt gaa gat gaa gaa gaa ggt gct ggc ggc ggt ggc ggt |
| Fully_optimized full | ggc ggt gaa ctg cgt ggc gag ggt gca acc gat agc cgt gca cat |
| length nucleotide | ggt gca ggc ggt ggc ggt ccg ggt cgt gct ggt tgc tgt ctg ggt |
| SEQ ID: 95 | aaa gct gtg cgc ggc gcg aaa ggt cat cat cat ccg cat ccg ccg |
| | gca gca ggt gca ggt gca gct ggc ggt gcg gaa gcc gat ctg aaa |
| | gcc ctg acc cat agc gtc ctg aaa aaa ctg aaa gaa cgt cag ctg |
| | gag ctg ctg ctg caa gca gta gaa agc cgt ggc ggt acc cgt acg |
| | gct tgt ctg ctg ctg ccg ggt cgt ctg gat tgc cgt ctg ggt ccg |
| | ggt gca ccg gct ggt gcg cag ccg gca caa ccg ccg agc agc tac |
| | agc ctg ccg ctg ctg ctg tgt aaa gtg ttt cgt tgg ccg gac ctg |
| | cgc cat agc agc gaa gtt aaa cgc ctg tgc tgt tgc gag agc tat |
| | ggc aaa att aac ccg gaa ctg gtt tgt tgc aat ccg cat cat ctg |
| | agc cgt ctg tgt gaa ctg gag agc ccg ccg ccg ccg tat agc cgt |
| | tac ccg ctg gat ttc ctg aaa ccg act gca gat tgc ccg gac gca |
| | gtc ccg agc agc gct gag acc ggc ggc acc aac tat ctg gca ccg |
| | ggc ggt ctg agc gat agc cag ctg ctg ctg gaa ccg ggc gac cgt |
| | agc cat tgg tgt gtg gtt gcc tat tgg gaa gag aaa acg cgt gtc |
| | ggt cgc ctg tac tgc gta cag gaa ccg agc ctg gat atc ttt tat |
| | gac ctg ccg cag ggc aat ggt ttc tgt ctg ggc caa ctg aac agc |
| | gat aat aaa agc cag ctg gtg caa aaa gtt cgc agc aaa att ggc |
| | tgc ggt atc cag ctg acc cgt gaa gtt gac ggt gtc tgg gta tat |
| | aac cgc agc agc tac ccg att ttt atc aaa agc gcc acc ctg gat |
| | aat ccg gac agc cgt acg ctg ctg gtc cat aaa gta ttt ccg ggc |
| | ttc agc atc aaa gcg ttc gat tac gag aaa gcc tac agc ctg cag |
| | cgc ccg aac gac cat gaa ttc atg cag caa ccg tgg acg ggt ttt |
| | act gtg cag atc agc ttc gtt aaa ggc tgg ggt caa tgc tac acc |
| | cgt cag ttt atc agc agc tgt ccg tgc tgg ctg gaa gtg att ttc |
| | aat agc cgc aag ggc gag ctc aat agc aag ctt gaa ggt aag cct |
| | atc cct aac cct ctc ctc ggt ctc gat agc acg tgagtcgac |
| Translation of 95 | GSGRKKRRQRRRGFRTKRSALVRRLWRSRAPGGEDEEEGAGGGGGGGELRGEGATDSRAH |
| Tat-Smad7 | GAGGGGPGRAGCCLGKAVRGAKGHHHPHPPAAGAGAAGGAEADLKALTHSVLKKLKERQL |
| M216L mutation | ELLLQAVESRGGTRTACLLLPGRLDCRLGPGAPAGAQPAQPPSSYSLPLLLCKVFRWPDL |
| Fully-optimized full | RHSSEVKRLCCCESYGKINPELVCCNPHHLSRLCELESPPPPYSRYPLDFLKPTADCPDA |
| length protein | VPSSAETGGTNYLAPGGLSDSQLLLEPGDRSHWCVVAYWEEKTRVGRLYCVQEPSLDIFY |
| SEQ ID: 96 | DLPQGNGFCLGQLNSDNKSQLVQKVRSKIGCGIQLTREVDGVWVYNRSSYPIFIKSATLD |
| | NPDSRTLLVHKVFPGFSIKAFDYEKAYSLQRPNDHEFMQQPWTGFTVQISFVKGWGQCYT |
| | RQFISSCPCWLEVIFNSRKGELNSKLEGKPIPNPLLGLDST |
| SEQ ID: 97 | ggatccggcc gtaaaaaacg ccgtcaacgc cgccgttcac attggtgtgt |
| | ggttgcctat tgggaagaga aaacgcgtgt cggtcgcctg tactgcgtac |
| | aggaaccgag cctggatatc ttttatgacc tgccgcaggg caatggtttc |
| | tgtctgggcc aactgaacag cgataataaa agccagctgg tgcaaaaagt |
| | tcgcagcaaa attggctgcg gtatccagct gacccgtgaa gttgacggtg |
| | tctgggtata taaccgcagc agctacccga tttttatcaa aagcgccacc |
| | ctggataatc cggacagccg tacgctgctg gtccataaag tatttccggg |
| | cttcagcatc aaagcgttcg attacgagaa agcctacagc ctgcagcgcc |
| | cgaacgacca tgaattcatg cagcaaccgt ggacgggttt tactgtgcag |
| | atcagcttcg ttaaaggctg gggtcaatgc aagggcgagc tcaatagcaa |
| | gcttgaaggt aagcctatcc ctaaccctct cctcggtctc gatagcacgt |
| | gagtcgac |
| Tat-C-Smad7-V5 | gga tcc ggc cgt aaa aaa cgc cgt caa cgc cgc cgt tca cat tgg |
| most optimized | tgt gtg gtt gcc tat tgg gaa gag aaa acg cgt gtc ggt cgc ctg |
| Includes V5 epitope | tac tgc gta cag gaa ccg agc ctg gat atc ttt tat gac ctg ccg |
| and the pET101- | cag ggc aat ggt ttc tgt ctg ggc caa ctg aac agc gat aat aaa |
| Topo backbone | agc cag ctg gtg caa aaa gtt cgc agc aaa att ggc tgc ggt atc |
| SEQ ID: 98 | cag ctg acc cgt gaa gtt gac ggt gtc tgg gta tat aac cgc agc |
| | agc tac ccg att ttt atc aaa agc gcc acc ctg gat aat ccg gac |
| | agc cgt acg ctg ctg gtc cat aaa gta ttt ccg ggc ttc agc atc |
| | aaa gcg ttc gat tac gag aaa gcc tac agc ctg cag cgc ccg aac |
| | gac cat gaa ttc atg cag caa ccg tgg acg ggt ttt act gtg cag |
| | atc agc ttc gtt aaa ggc tgg ggt caa tgc tac acc cgt cag ttt |
| | atc agc agc tgt ccg tgc tgg ctg gaa gtg att ttc aat agc cgc |
| | aag ggc gag ctc aat agc aag ctt gaa ggt aag cct atc cct aac |
| | cct ctc ctc ggt ctc gat agc acg tgagtcgac |
| Tat-Smad7 | gga tcc ggt cgt aaa aaa cgt cgt cag cgt cgt cgt ggt ttc cgt |
| M216L | acc aaa cgt tct gcg ctg gtt cgt cgt ctg tgg cgt tct cgt gcg |
| Optimized | ccg ggt ggt gaa gac gaa gaa gaa ggt gcg ggt ggt ggt ggt ggt |
| SEQ ID: 99 | ggt ggt gaa ctg cgt ggt gaa ggt gcg acc gac tct cgt gcg cac |
| | ggt gcg ggt ggt ggt ggt ccg ggt cgt gcg ggt tgc tgc ctg ggt |
| | aaa gcg gtt cgt ggt gcg aaa ggt cac cac cac ccg cac ccg ccg |
| | gcg gcg ggt gcg ggt gcg gcg ggt ggt gcg gaa gcg gac ctg aaa |
| | gcg ctg acc cac tct gtt ctg aaa aaa ctg aaa gaa cgt cag ctg |
| | gaa ctg ctg ctg cag gcg gtt gaa tct cgt ggt ggt acc cgt acc |
| | gcg tgc ctg ctg ctg ccg ggt cgt ctg gac tgc cgt ctg ggt ccg |
| | ggt gcg ccg gcg ggt gcg cag ccg gcg cag ccg ccg tct tct tac |
| | tct ctg ccg ctg ctg ctg tgc aaa gtt ttc cgt tgg ccg gac ctg |
| | cgt cac tct tct gaa gtt aaa cgt ctg tgc tgc tgc gaa tct tac |
| | ggt aaa atc aac ccg gaa ctg gtt tgc tgc aac ccg cac cac ctg |
| | tct cgt ctg tgc gaa ctg gaa tct ccg ccg ccg ccg tac tct cgt |
| | tac ccg ctg gac ttc ctg aaa ccg acc gcg gac tgc ccg gac gcg |
| | gtt ccg tct tct gcg gaa acc ggt ggt acc aac tac ctg gcg ccg |
| | ggt ggt ctg tct gac tct cag ctg ctg ctg gaa ccg ggt gac cgt |
| | tct cac tgg tgc gtt gtt gcg tac tgg gaa gaa aaa acc cgt gtt |
| | ggt cgt ctg tac tgc gtt cag gaa ccg tct ctg gac atc ttc tac |
| | gac ctg ccg cag ggt aac ggt ttc tgc ctg ggt cag ctg aac tct |
| | gac aac aaa tct cag ctg gtt cag aaa gtt cgt tct aaa atc ggt |
| | tgc ggt atc cag ctg acc cgt gaa gtt gac ggt gtt tgg gtt tac |
| | aac cgt tct tct tac ccg atc ttc atc aaa tct gcg acc ctg gac |
| | aac ccg gac tct cgt acc ctg ctg gtt cac aaa gtt ttc ccg ggt |
| | ttc tct atc aaa gcg ttc gac tac gaa aaa gcg tac tct ctg cag |
| | cgt ccg aac gac cac gaa ttc atg cag cag ccg tgg acc ggt ttc |
| | acc gtt cag atc tct ttc gtt aaa ggt tgg ggt cag tgc tac acc |
| | cgt cag ttc atc tct tct tgc ccg tgc tgg ctg gaa gtt atc ttc |
| | aac tct cgt ggt aaa ccg atc ccg aac ccg ctg ctg ggt ctg gac |
| | tct acc tgagtcgac |
| Translation of 99 | GSGRKKRRQRRRGFRTKRSALVRRLWRSRAPGGEDEEEGAGGGGGGGELRGEGATDSRAH |
| SEQ ID: 100 | GAGGGGPGRAGCCLGKAVRGAKGHHHPHPPAAGAGAAGGAEADLKALTHSVLKKLKERQL |
| | ELLLQAVESRGGTRTACLLLPGRLDCRLGPGAPAGAQPAQPPSSYSLPLLLCKVFRWPDL |
| | RHSSEVKRLCCCESYGKINPELVCCNPHHLSRLCELESPPPPYSRYPLDFLKPTADCPDA |
| | VPSSAETGGTNYLAPGGLSDSQLLLEPGDRSHWCVVAYWEEKTRVGRLYCVQEPSLDIFY |
| | DLPQGNGFCLGQLNSDNKSQLVQKVRSKIGCGIQLTREVDGVWVYNRSSYPIFIKSATLD |
| | NPDSRTLLVHKVFPGFSIKAFDYEKAYSLQRPNDHEFMQQPWTGFTVQISFVKGWGQCYT |
| | RQFISSCPCWLEVIFNSRGKPIPNPLLGLDST |
| SEQ ID: 101 | ggatccggtc gtaaaaaacg tcgtcagcgt cgtcgtggtt tccgtaccaa |
| | acgttctgcg ctggttcgtc gtctgtggcg ttctcgtgcg ccgggtggtg |
| | aagacgaaga agaaggtgcg ggtggtggtg gtggtggtgg tgaactgcgt |
| | ggtgaaggtg cgaccgactc tcgtgcgcac ggtgcgggtg gtggtggtcc |
| | gggtcgtgcg ggttgctgcc tgggtaaagc ggttcgtggt gcgaaaggtc |
| | accaccaccc gcacccgccg gcggcgggtg cgggtgcggc gggtggtgcg |
| | gaagcggacc tgaaagcgct gacccactct gttctgaaaa aactgaaaga |
| | acgtcagctg gaactgctgc tgcaggcggt tgaatctcgt ggtggtaccc |
| | gtaccgcgtg cctgctgctg ccgggtcgtc tggactgccg tctgggtccg |
| | ggtgcgccgg cgggtgcgca gccggcgcag ccgccgtctt cttactctct |
| | gccgctgctg ctgtgcaaag ttttccgttg gccggacctg cgtcactctt |
| | ctgaagttaa acgtctgtgc tgctgcgaat cttacggtaa aatcaacccg |
| | gaactggttt gctgcaaccc gcaccacctg tctcgtctgt gcgaactgga |
| | atctccgccg ccgccgtact ctcgttaccc gctggacttc ctgaaaccga |
| | ccgcggactg cccggacgcg gttccgtctt ctgcggaaac cggtggtacc |
| | aactacctgg cgccgggtgg tctgtctgac tctcagctgc tgctggaacc |
| | gggtgaccgt tctcactggt gcgttgttgc gtactgggaa gaaaaaaccc |
| | gtgttggtcg tctgtactgc gttcaggaac cgtctctgga catcttctac |
| | gacctgccgc agggtaacgg tttctgcctg ggtcagctga actctgacaa |
| | caaatctcag ctggttcaga aagttcgttc taaaatcggt tgcggtatcc |
| | agctgacccg tgaagttgac ggtgtttggg tttacaaccg ttcttcttac |
| | ccgatcttca tcaaatctgc gaccctggac aacccggact ctcgtaccct |
| | gctggttcac aaagttttcc cgggtttctc tatcaaagcg ttcgactacg |
| | aaaaagcgta ctctctgcag cgtccgaacg accacgaatt catgcagcag |
| | ccgtggaccg gtttcaccgt tcagatctct ttcgttaaag gttggggtca |
| | gtgctacacc cgtcagttca tctcttcttg cccgtgctgg ctggaagtta |
| | tcttcaactc tcgtggtaaa ccgatcccga acccgctgct gggtctggac |
| | tctacctgag tcgac |
| SEQ ID: 102 | ggatccggcc gtaaaaaacg ccgtcaacgc cgccgtggtt tccgtacgaa |
| | acgcagcgcc ctggtccgtc gcctgtggcg cagccgtgct ccgggtggtg |
| | aagatgaaga agaaggtgct ggcggcggtg gcggtggcgg tgaactgcgt |
| | ggcgagggtg caaccgatag ccgtgcacat ggtgcaggcg gtggcggtcc |
| | gggtcgtgct ggttgctgtc tgggtaaagc tgtgcgcggc gcgaaaggtc |
| | atcatcatcc gcatccgccg gcagcaggtg caggtgcagc tggcggtgcg |
| | gaagccgatc tgaaagccct gacccatagc gtcctgaaaa aactgaaaga |
| | acgtcagctg gagctgctgc tgcaagcagt agaaagccgt ggcggtaccc |
| | gtacggcttg tctgctgctg ccgggtcgtc tggattgccg tctgggtccg |
| | ggtgcaccgg ctggtgcgca gccggcacaa ccgccgagca gctacagcct |
| | gccgctgctg ctgtgtaaag tgtttcgttg gccggacctg cgccatagca |
| | gcgaagttaa acgcctgtgc tgttgcgaga gctatggcaa aattaacccg |
| | gaactggttt gttgcaatcc gcatcatctg agccgtctgt gtgaactgga |
| | gagcccgccg ccgccgtata gccgttaccc gctggatttc ctgaaaccga |
| | ctgcagattg cccggacgca gtcccgagca gcgctgagac cggcggcacc |
| | aactatctgg caccgggcgg tctgagcgat agccagctgc tgctggaacc |
| | gggcgaccgt agccattggt gtgtggttgc ctattgggaa gagaaaacgc |
| | gtgtcggtcg cctgtactgc gtacaggaac cgagcctgga tatcttttat |
| | gacctgccgc agggcaatgg tttctgtctg ggccaactga acagcgataa |
| | taaaagccag ctggtgcaaa aagttcgcag caaaattggc tgcggtatcc |
| | agctgacccg tgaagttgac ggtgtctggg tatataaccg cagcagctac |
| | ccgattttta tcaaaagcgc caccctggat aatccggaca gccgtacgct |
| | gctggtccat aaagtatttc cgggcttcag catcaaagcg ttcgattacg |
| | agaaagccta cagcctgcag cgcccgaacg accatgaatt catgcagcaa |
| | ccgtggacgg gttttactgt gcagatcagc ttcgttaaag gctggggtca |
| | atgctacacc cgtcagttta tcagcagctg tccgtgctgg ctggaagtga |
| | ttttcaatag ccgcaagggc gagctcaata gcaagcttga aggtaagcct |
| | atccctaacc ctctcctcgg tctcgatagc acgtgagtcg ac |
| Optimized | gacgttgtat acgactccta tagggcggcc gggaattcgt cgactggatc |
| mammalian GST- | cggtaccgag gagatctgcc gccgcgatcg ccatgtcccc cattctgggc |
| Tat-Smad7-myc- | tactggaaga ttaagggcct ggtgcagcct actagactgc tgctggaata |
| flag | cctggaggaa aaatatgaag agcatctgta tgaaagagac gagggggata |
| SEQ ID: 103 | aatggaggaa caagaaattc gaactgggac tggagtttcc taatctgcca |
| | tactatattg acggcgatgt gaagctgact cagtctatgg ctatcattag |
| | atacatcgca gacaaacaca acatgctggg cgggtgtcct aaggaaaggg |
| | cagagattag tatgctggag ggagccgtgc tggatattag atacggcgtc |
| | tcacgcatcg cctatagcaa agacttcgaa accctgaagg tggattttct |
| | gagcaaactg cctgaaatgc tgaagatgtt cgaggacaga ctgtgccaca |
| | aaacctacct gaatggcgac catgtcacac acccagattt tatgctgtac |
| | gacgccctgg atgtggtcct gtatatggac cccatgtgtc tggatgcttt |
| | ccctaagctg gtgtgcttta agaaaaggat cgaggcaatt ccccagatcg |
| | ataagtacct gaaaagctcc aagtatatcg cttggcctct ccagggctgg |
| | caggcaacat tcggaggcgg ggaccatccc cctaaaagcg acctggaggt |
| | gctgtttcag ggaccactgg gcagcggccg gaagaagcgg cggcagaggc |
| | gccgaagtag gttccgcact aagcggtcag cactggtgcg gagactgtgg |
| | cgatctcggg ctcctggagg agaggacgag gaagagggag caggcggcgg |
| | cggaggagga ggagaactgc gcggggaggg agctacagat agccgagccc |
| | acggagctgg aggaggagga ccagggcgag ccggatgctg tctgggcaaa |
| | gcagtgagag gcgccaaggg gcaccatcac ccccatccac ccgccgctgg |
| | cgcaggagca gccggcggag ctgaggcaga cctgaaagcc ctgactcaca |
| | gtgtgctgaa gaaactgaag gaaagacagc tggagctgct gctccaggca |
| | gtcgaatcac gcggaggcac ccgaacagct tgtctgctgc tgcccggccg |
| | gctggactgc cggctgggac ccggcgcccc tgctggggca cagccagccc |
| | agcctccatc tagttatagc ctgcccctgc tgctgtgtaa ggtgttccga |
| | tggcctgatc tgcggcattc aagcgaagtc aaaaggctgt gctgttgcga |
| | gtcctacggc aagattaacc cagaactggt gtgttgcaat ccccatcacc |
| | tgtctcgact gtgtgaactg gagtcccccc ctccacccta ctctaggtat |
| | cctatggact ttctgaagcc aaccgctgac tgcccagatg cagtgccctc |
| | ctctgccgag actgggggaa ccaactacct ggctcctggc ggactgagcg |
| | actcccagct gctgctggaa ccaggggatc gcagccactg gtgtgtggtc |
| | gcctactggg aagagaagac aagagtggga aggctgtatt gcgtccagga |
| | gccttccctg gacatcttct acgatctgcc acaggggaat ggattttgtc |
| | tgggccagct gaactctgac aataagagtc agctggtgca gaaagtccgg |
| | agcaagattg gctgcggcat ccagctgacc agggaggtgg acggcgtgtg |
| | ggtctacaac cgcagttcat atccaatctt catcaagagc gccactctgg |
| | acaatcccga ttcccgcacc ctgctggtgc ataaggtctt ccccggcttc |
| | agcatcaagg ccttcgacta cgagaaggct tatagtctcc agcggcccaa |
| | cgatcacgag ttcatgcagc agccttggac aggcttcact gtgcagatca |
| | gcttcgtcaa gggatggggc cagtgctaca caaggcagtt catctcaagc |
| | tgtccctgtt ggctggaagt cattttcaat agtaggacgc gtacgcggcc |
| | gctcgagcag aaactcatct cagaagagga tctggcagca aatgatatcc |
| | tggattacaa ggatgacgac gataaggttt aa |
| codon optimized | tcacccccat ccacccgccg ctggcgcagg agcagccggc ggagctgagg |
| hSmad7-myc-Flag | cagacctgaa agccctgact cacagtgtgc tgaagaaact gaaggaaaga |
| for mammalian | cagctggagc tgctgctcca ggcagtcgaa tcacgcggag gcacccgaac |
| expression | agcttgtctg ctgctgcccg gccggctgga ctgccggctg ggacccggcg |
| SEQ ID: 104 | cccctgctgg ggcacagcca gcccagcctc catctagtta tagcctgccc |
| | ctgctgctgt gtaaggtgtt ccgatggcct gatctgcggc attcaagcga |
| | agtcaaaagg ctgtgctgtt gcgagtccta cggcaagatt aacccagaac |
| | tggtgtgttg caatccccat cacctgtctc gactgtgtga actggagtcc |
| | ccccctccac cctactctag gtatcctatg gactttctga agccaaccgc |
| | tgactgccca gatgcagtgc cctcctctgc cgagactggg ggaaccaact |
| | acctggctcc tggcggactg agcgactccc agctgctgct ggaaccaggg |
| | gatcgcagcc actggtgtgt ggtcgcctac tgggaagaga agacaagagt |
| | gggaaggctg tattgcgtcc aggagccttc cctggacatc ttctacgatc |
| | tgccacaggg gaatggattt tgtctgggcc agctgaactc tgacaataag |
| | agtcagctgg tgcagaaagt ccggagcaag attggctgcg gcatccagct |
| | gaccagggag gtggacggcg tgtgggtcta caaccgcagt tcatatccaa |
| | tcttcatcaa gagcgccact ctggacaatc ccgattcccg caccctgctg |
| | gtgcataagg tcttccccgg cttcagcatc aaggccttcg actacgagaa |
| | ggcttatagt ctccagcggc ccaacgatca cgagttcatg cagcagcctt |
| | ggacaggctt cactgtgcag atcagcttcg tcaagggatg gggccagtgc |
| | tacacaaggcagttcatctcaagctgtccctgttggctgg aagtcattttcaa |
| Optimized | gaattcgtcg actggatccg gtaccgagga gatctgccgc cgcgatcgcc |
| mammalian GST- | atgtccccca ttctgggcta ctggaagatt aagggcctgg tgcagcctac |
| Tat-C-Smad7-myc- | tagactgctg ctggaatacc tggaggaaaa atatgaagag catctgtatg |
| flag | aaagagacga gggggataaa tggaggaaca agaaattcga actgggactg |
| SEQ ID: 105 | gagtttccta atctgccata ctatattgac ggcgatgtga agctgactca |
| | gtctatggct atcattagat acatcgcaga caaacacaac atgctgggcg |
| | ggtgtcctaa ggaaagggca gagattagta tgctggaggg agccgtgctg |
| | gatattagat acggcgtctc acgcatcgcc tatagcaaag acttcgaaac |
| | cctgaaggtg gattttctga gcaaactgcc tgaaatgctg aagatgttcg |
| | aggacagact gtgccacaaa acctacctga atggcgacca tgtcacacac |
| | ccagatttta tgctgtacga cgccctggat gtggtcctgt atatggaccc |
| | catgtgtctg gatgctttcc ctaagctggt gtgctttaag aaaaggatcg |
| | aggcaattcc ccagatcgat aagtacctga aaagctccaa gtatatcgct |
| | tggcctctcc agggctggca ggcaacattc ggaggcgggg accatccccc |
| | taaaagcgac ctggaggtgc tgtttcaggg accactgggc agcggccgga |
| | agaagcggcg gcagaggcgc agccactggt gtgtggtcgc ctactgggaa |
| | gagaagacaa gagtgggaag gctgtattgc gtccaggagc cttccctgga |
| | catcttctac gatctgccac aggggaatgg attttgtctg ggccagctga |
| | actctgacaa taagagtcag ctggtgcaga aagtccggag caagattggc |
| | tgcggcatcc agctgaccag ggaggtggac ggcgtgtggg tctacaaccg |
| | cagttcatat ccaatcttca tcaagagcgc cactctggac aatcccgatt |
| | cccgcaccct gctggtgcat aaggtcttcc ccggcttcag catcaaggcc |
| | ttcgactacg agaaggctta tagtctccag cggcccaacg atcacgagtt |
| | catgcagcag ccttggacag gcttcactgt gcagatcagc ttcgtcaagg |
| | gatggggcca gtgctacaca aggcagttca tctcaagctg tccctgttgg |
| | ctggaagtca ttttcaatag taggacgcgt acgcggccgc tcgagcagaa |
| | actcatctca gaagaggatc tggcagcaaa tgatatcctg gattacaagg |
| | atgacgacga taaggtttaa |
| SEQ ID: 106 | GLPIPNPLLGLDS |

### BRIEF DESCRIPTION OF THE DRAWINGS

The following drawings form part of the present specification and are included to further demonstrate certain embodiments of the present technology. The embodiments may be better understood by reference to one or more of these drawings in combination with the detailed description of specific embodiments presented herein.

FIG. 1A depicts exemplary Smad7 protein fusion constructs useful in the methods of this disclosure. As indicated, these fusion proteins may include a fusion protein partner, for example, GST, MBP, or Trx; a protein transduction domain (PTD), such as Tat or PEP; a tag specific for antibody staining or Western blotting, such as HA or V5; and a tag for protein purification, such as 6×His. FIG. 1B illustrates PY-C-Smad7 fusion protein functional domains and predicted functions.

FIGS. 2A-2D demonstrate the treatment effects of Tat-PY-C-Smad7 on oral mucositis in mice. Oral mucositis was induced by 18Gy craniofacial radiation. Tat-Smad7(203-426), 1 μg/mouse in 50% glycerol (treated) or 50% glycerol alone (control), was administered daily to the oral cavity starting on day 6 after irradiation until day 10. Mice were euthanized on day 10, and tongues were analyzed by histology. FIG. 2A the left panels show H&E stained histological sections of treated and control tongues showing ulcer size reduction and reduced inflammation in pathology using Tat-PY-C-Smad7 (dotted lines highlight ulceration). FIG. 2A the right panel shows a graphical representation of ulcer size reduction in treated skin (p=0.0079). FIG. 2B the left panels show epithelial proliferation in tongue sections from control and treated animals using antibodies against BrdU (white) and keratin 14 (gray) staining. BrdU+epithelials cells increased using Tat-PY-C-Smad7 (K14: keratin 14). FIG. 2C right panel shows quantification of the increased number of BrdU+ cells/mm in the basement membrane in treated compared with control cells (p=0.0070). FIG. 2C left panels show pH2AX(white)/DAPI(gray) stained tongue sections from control and treated mice demonstrating reduced DNA damaged (pH2AX+) cells by Tat-PY-C-Smad7 (dotted lines highlight epithelial/stromal boundary). FIG. 2C the right panel shows quantification of the reduced number of pH2AX+ cells/mm in the basement membrane in treated compared with control cells (p=0.0232). FIG. 2D left panels show pSmad2(white)/DAPI(gray) stained tongue sections from control and treated mice demonstrating reduced phosphorylation of Smad2 (pSmad2) treated with Tat-Smad7(203-426). Dotted lines highlight epithelial/stromal boundary. FIG. 2D right panel shows quantification of the reduced number of pSmad2+ cells/mm in the basement membrane in treated compared with control cells (p=0.0142). Scale bar: 150 μm in FIG. 2A, 50 μm in FIGS. 2B, 2C, and 2D.

FIGS. 3A and B show the treatment effects of Tat-PY-C-Smad7 on radiation induced dermatitis. Dermatitis was induced by 35Gy radiation to the legs of mice. Tat-Smad7(203-426), 1 μg/mouse, was injected subcutaneously 3 times/week, starting on day 16 after radiation. Skin biopsy was harvested on day 35. FIG. 3A shows H&E staining of skin from control mice treated with PBS and on day 35 (D35). FIG. 3B shows H&E staining of skin from mice treated with Tat-Smad7(203-426). Scale bar: 200 μm.

FIGS. 4A-4C show Tat-PY-C-Smad7 alleviated skin inflammation in K5.TGFβ1 transgenic mice, a mouse model of psoriasis. In the presence of exogenous pathogens, K5.TGFβ1 transgenic mice are also a model for contact dermatitis or atopic dermatitis. K5.TGFβ1 skin was treated with Tat-PY-C-Smad7 (s.c. 2 μg/mouse, 3×/wk) in two groups of K5.TGFβ1 mice, and photographs were taken daily. Skin biopsies were harvested on day 13. FIG. 4A shows photographs from Days 0, 3, 6, and 10). Tat-Smad7(203-426) treated skin showed obvious gross alleviation from inflammation. FIG. 4B shows H&E stained sections of skin from day 0 and day 13. Prior to treatment (day 0), epidermis showed signficant hyperplasia induced by massive infiltrated leukocytes and hyperproliferative fibroblasts. These skin phenotypes worsen without treatment. Tat-PY-C-Smad7 treated skin reduced epidermal hyperplasia, inflammation and fibrotic response. Scale bar: 100 μm. FIG. 4C left panel shows that K5.TGFβ1 transgenic mouse skin before treatment (day 0) had numerous mast cells in the dermis (Giemsa staining), the type of immune cells that release histamine to induce allergic/atopic dermatitis. FIG. 4C right panel shows that Tat-Smad7 (203-426) treatment reduced the number of mast cells in K5.TGFβ1 skin on day 13 treated with Tat-Smad7 (203-426). Skin biopsies were taken prior to initiating treatment (day 0, FIG. 4C left panel) and again on day 13 (FIG. 4C right panel).

FIG. 5 shows that Tat-Smad7(203-426) alleviated Imiquimod-induced inflammation in mouse ear skin, a mouse model for psoriasis. Skin exposed to Imiquimod can also develop severe contact dermatitis. 5% Imiquimod cream was applied to mouse ear daily. One hour later was treated topically with either PBS (vehicle control) or Tat-Smad7(203-426), 1 μg, daily. Ear biopsy was taken on day 6. FIG. 5A shows H&E staining of skin sections. Sections from the PBS treated ear (left panel) showed significant epidermal hyperplasia and numerous inflammatory cells in the stroma induced by Imiquimod. Sections from the Tat-Smad7(203-426) treated ear (right panel) had no epidermal hyperplasia or inflammation. FIG. 5B shows Giemsa staining for mast cells (dark purple cells in the dermis) in skin sections. Mast cells are numerous in sections from the PBS treated ear (left panel), but there are only a few in sections from the Tat-Smad7 (203-426) treated ear (right panel). Scale bar: 100 μm.

FIG. 6 shows a dose-dependent effect of Tat-Smad7(203-426) on human fibroblast expansion. Primary human skin fibroblasts were cultured with Tat-Smad7(203-426) at 2.5 μg/ml, 5 μg/ml, and 7.5 μg/ml or with vehicle (PBS) alone. 5 μg/ml and 7.5 μg/ml reduced fibroblast expansion (P<0.0001 for each compared to control), consistent with Tat-Smad7(203-426)'s anti-fibroblast activation and anti-fibrosis effect in vivo.

FIG. 7 shows the effect of treatment with Tat-Smad7(203-426) on wounds in diabetic mice. Tat-Smad7(203-426) or the vehicle control (PBS) was applied topically, 1 μg/wound, every other day, beginning immediately after punch biopsies (day 0). Photographs were taken every other day. Biopsies of the healing wounds were taken on Day 10. Photographs from days 0, 2, 4, 6, 8, and 10 show examples of the gross appearance of 6-mm excisional wounds in control and treated mice (upper panels). No apparent healing was seen by day 10 in PBS treated wounds but healing is seen in Tat-Smad7(203-426) treated wounds.

FIGS. 8A-8C show the treatment effect of the fusion protein construct composed of full length human Smad7 fused to Tat (Tat-Smad7) on radiation-induced dermatitis in mice. Dermatitis was induced by 35Gy radiation to the legs of mice. Tat-Smad7, 1 mg/mouse, was injected subcutaneously 3 times/week, starting on day 7 after radiation. Dermatitis severity was assessed daily following treatment using scoring criteria established by the Cancer Therapy Evaluation Program, i.e., 1 being mildest, 5 being most severe with ulcer. Skin biopsies were harvested on day 18. FIG. 8A provides a graphical representation of dermatitis scores showing treated lesions have less severe dermatitis by Day 16 (*; p<0.05). FIG. 8B shows immunofluorescence staining of Day 18 skin sections from control (PBS) and treated (Tat-Smad7) skin using an antibody specific for Tat-Smad7 to show that Tat-Smad7 (light gray) penetrated into both dermal cells and epidermal cells. K14 antibody staining (dark gray) highlights the epidermis. FIG. 8C shows H&E stained Day 18 skin sections showing that treated skin (Tat-Smad7) had healed ulcers and reductions in inflammation and fibrotic response compared with vehicle control (PBS). Vertical lines highlight unhealed ulcer in PBS control. Dotted lines highlight healed ulcer treated by Tat-Smad7.

FIGS. 9A and 9B show that full length Tat-Smad7 reduced the number of mast cells prior to complete reversal of skin inflammation in K5.TGFβ1 transgenic mice, a mouse model for psoriasis which can also exhibit atopic dermatitis. FIG. 9, left panel shows numerous mast cells in the K5.TGFβ1 transgenic mouse skin before treatment (day 0). FIG. 9, right panel shows that full length Tat-Smad7 reduced the number of mast cells prior to complete reversal of skin inflammation in K5.TGFβ1 transgenic mice. K5.TGFβ1 skin was treated with Tat-Smad7, s.c. 1 μg/mouse, on days −1, −3, and −5. Skin biopsies were taken prior to initiating treatment (day 0, FIG. 9, left panel) and again on day 6 (FIG. 9, right panel), prior to reversal of epidermal hyperplasia and inflammation. Mast cells can be seen as dark gray (toluidine blue) staining in the sections. Scale bar: 100 μm.

FIG. 10 shows that Tat-Smad7(203-217) alleviated TGFβ1-induced skin inflammation and fibrosis. Tat-Smad7(203-217) peptide was injected s.c. daily, 15 μg in phosphate buffered saline (PBS), into the skin of K5.TGFβ1 mice. Untreated K5.TGFβ1 skin progressively deteriorates, and treatment with vehicle (PBS) alone does not alleviate the phenotype. Skin biopsies were taken before (Day 0) and after treatment (Day 18). Inflamed skin gradually improved macroscopically beginning 2 weeks after treatment and was grossly obvious by 3 weeks (upper panels; “Gross”). On histology (H&E stained sections), peptide-treated skin showed reductions in leukocyte infiltration and epidermal hyperplasia (middle panels; “Histology”). Additionally, peptide treatment reduced alpha-smooth muscle actin (αSMA), a marker of activated fibroblasts (bottom panels, “αSMA”).

FIG. 11 shows that Tat-Smad7(203-217) promoted healing of RT-induced skin wounds and ulcers in mice. Daily treatment started on day 10 after RT (30Gy on the leg). The gross (upper panels), histological (H&E stained; middle panels), and immunofluorescence-labelled (pH2AX/K14; bottom panels) show the effect of treatment with the control peptide, Tat, 15 μg/day, as compared with 30 μg/day Tat-Smad7(203-217) s.c. starting on day 10 after irradiation. Skin biopsies were taken on Day 26. Dotted lines highlight ulcer in control peptide (Tat alone)-treated skin but not in Tat-Smad7 (203-217) treated skin (middle panels). The skin was thicker (due to fibrosis and inflammation) in control peptide-treated skin compared with Tat-Smad7 (203-217)-treated skin. Cells with nuclear pH2AX (light gray), a DNA damage marker, were fewer in Tat-Smad7(203-217) treated skin sections than control peptide treated skin sections (bottom pannels). K14 (dark gray) is a counterstain to highlight epithelial layers of the skin (bottom pannels).

FIG. 12 shows Tat-Smad7(203-217) alleviated Imiquimod-induced inflammation in mouse ear skin, a mouse model for psoriasis. Skin exposed to Imiquimod can also develop severe contact dermatitis. Each day, 5% Imiquimod cream was applied to mouse ear, and one hour later, the ear was treated topically with or without Tat-Smad7(203-217), 30 μg. An ear biopsy was taken on day 6. Giemsa staining for mast cells (dark gray cells in the dermis) showed numerous mast cells in the Imiquimod-treated ear but few mast cells in the Tat-Smad7(203-217)-treated ear. Epidermal hyperplasia was not reversed at this stage, but could be seen after further treatment based on data in presented in FIGS. 10 and 11. Scale bar: 100 μm.

FIG. 13 shows that Tat-Smad7(259-426) alleviated TGFβ1-induced skin inflammation and fibrosis. Tat-Smad7(259-426) was injected s.c. 3×/wk, 1 μg in phosphate buffered saline (PBS), into the skin of K5.TGFβ1 mice. Untreated K5.TGFβ1 skin progressively deteriorates. Skin biopsies were taken before (Day 0) and after treatment (Day 19). Inflamed skin gradually improved after treatment and was grossly obvious by 3 wks (top panels). On histology (H&E stained sections), treated skin showed significant reductions in leukocyte infiltration and fibrosis (lower panels).

### DETAILED DESCRIPTION

As further described herein, the disclosure provides, inter alia, Protein Transduction Domain (PTD)-Smad7 fusion proteins and biologically active fragments and derivatives thereof (including but not limited to, PTD-Smad7(203-426), PTD-Smad7(259-426), and PTD-Smad7(203-217), nucleic acids encoding such proteins, vectors including such nucleic acids, and cells encompassing the vectors, nucleic acids, and/or proteins all for use in formulating medicaments and for treating and/or preventing one or more diseases or disorders. Also provided are methods for making and for screening PTD-Smad7 proteins and biologically active fragments and derivatives thereof useful for treating and/or preventing one or more diseases or disorders. Also provided are methods for predicting and/or evaluating a response to treatment using one or more markers associated with exposure to PTD-Smad7 and biologically active fragments and derivatives thereof. Such markers may include, but are not limited to, Rac1 for cell migration, NF-κB for inflammation, and TGF-β for growth arrest and inflammation.

PTD-Smad7 and biologically active fragments and derivatives thereof (including but not limited to, PTD-Smad7(203-426), PTD-Smad7(259-426), and PTD-Smad7(203-217) treatable diseases and disorders may include those including one or more of reduced cell proliferation, reduced cell migration, increased cell death, excessive inflammation, radiation damage, and/or DNA damage. PTD-Smad7 treatable diseases and disorders may include those where treatment with a PTD-Smad7 protein and biologically active fragments and derivatives thereof (including but not limited to, PTD-Smad7(203-426), PTD-Smad7(259-426), and PTD-Smad7(203-217) that have one or more activities including but not limited to increasing proliferation, reducing or inhibiting cell death, reducing excessive inflammation, preventing DNA damage, and/or increasing cell migration. Such diseases and/or disorders may include but are not limited to acute (e.g., through surgery, combat, trauma, diabetes) and chronic wounds (e.g., ulcers, such as diabetic, pressure, venous), scarring (including keloid and hypertrophic scarring), fibrosis including lung fibrosis and radiation-induced lung fibrosis in lung cancer patients, and aberrant healing, mucositis (e.g., oral and/or gastro-intestinal, and cancer treatment-induced oral mucositis), stomatitis including recurrent aphthous stomatitis (Canker sore), proctitis, autoimmune disease (e.g., psoriasis, arthritis), radiation-induced dermatitis (radiodermatitis or RT dermatitis), atopic dermatitis, contact dermatitis, allergic dermatitis, Interstitial lung fibrosis (ILF), and Radiation Pneumonitis leading to Pulmonary Fibrosis (e.g. due to cancer treating radiation).

It is critical for oral mucositis prevention and treatment to overcome epithelial ablation due to massive apoptosis and blunted keratinocyte proliferation. The proliferative and anti-apoptotic effects of PTD-Smad7 are more obvious in oral mucositis than in normal oral mucosa, when TGF-β1, a potent growth inhibitor and apoptosis inducer for epithelial cells, was increased.

Dampening excessive inflammation creates a microenvironment for oral mucositis healing. The antagonistic effect of PTD-Smad7 and biologically active fragments and derivatives thereof (including, but not limited to, PTD-Smad7(203-426), PTD-Smad7(259-426), and PTD-Smad7(203-217) on both TGF-β and NF-κB signaling makes PTD-Smad7 a more efficient anti-inflammatory molecule than other agents targeting only NF-κB. Because inflammatory cells produce cytokines that further activate TGF-β and NF-κB, reduced TGF-β and NF-κB signaling, found in K5.Smad7, K5.Smad7(203-426) or PTD-Smad7 or PTD-Smad7(203-426) treated oral mucosa after radiation, reflects the direct antagonistic effect of Smad7 on these two pathways and the consequence of reduced inflammatory cytokines from infiltrated leukocytes. However, PTD-Smad7 did not reduce NF-κB or TGF-β signaling below their normal physiological conditions. This incomplete blockade of NF-κB or TGF-β signaling may be beneficial to oral mucositis healing, as a complete loss of either pathway could induce excessive inflammation.

The primary obstacle to using growth factors to treat oral mucositis in cancer patients is the potential risk of promoting cancer cell growth. The majority of human oral cancers lose TGF-β signaling in tumor epithelial cells. Thus, anti-Smad-associated cell proliferation and migration by PTD-Smad7 would not be effective in cancer cells. In tumors with intact TGF-β signaling, activation of other oncogenic pathways could override TGF-β-induced tumor suppressive effects. These two scenarios could explain why there was no observation of PTD-Smad7 increasing proliferation and migration in oral cancer cells with mutant or intact TGF-β signaling components.

Additionally, TGF-β signaling promotes tumor invasion mainly through Smad-independent mechanisms after loss of TGF-β-induced tumor suppression. Thus, blocking TGF-β signaling by PTD-Smad7 in cancer cells could abrogate TGF-β-mediated tumor promotion effects, which behaves similarly to TGF-β inhibitors currently being used in clinical trials for advanced cancers. Further, the potent anti-inflammatory effect of PTD-Smad7 may reduce the risk of tumor progression. Therefore, long-term PTD-Smad7(203-426) or Tat-Smad7(203-426) application may also be helpful in cancer treatment.

Spontaneous tumor formation in K5. Smad7 mice has not been observed. Because Smad7 is not a secreted protein, local and short-term PTD-Smad7 and biologically active fragments and derivatives thereof (including but not limited to, PTD-Smad7(203-426), PTD-Smad7(259-426), and PTD-Smad7(203-217) protein delivery in oral mucositis treatment should have few systemic effects. In bone marrow transplant patients, whose oral epithelia do not contain cancer cells, PTD-Smad7 and biologically active fragments and derivatives thereof (including but not limited to, PTD-Smad7(203-426), PTD-Smad7(259-426), and PTD-Smad7(203-217) topical application may be suitable for both prevention and treatment of oral mucositis.

Although not wishing to be bound by any theory, PTD-Smad7- and biologically active fragments and derivatives thereof (including but not limited to, PTD-Smad7(203-426), PTD-Smad7(259-426), and PTD-Smad7(203-217) mediated oral mucositis healing appears to be a result of targeting multiple pathogenic processes mediated by one or more molecules, similarly to Smad7 (see, e.g., Han G, Bian L, Li F, Cotrim A, Wang D, Lu J, et al. Preventive and therapeutic effects of Smad7 on radiation-induced oral mucositis. Nat Med. 2013; 19(4):421-8; U.S. patent application Ser. No. 14/773,167, Publication No. US 2016/0039894, which is incorporated herein in its entirety). It is believed that one or more of these molecules (e.g., TGF-β, NF-κB, CtBP1, Rac1) may also be helpful as predictive and therapeutic responsive markers of oral mucositis in patients.

A. Nucleic acids, Vectors and Host Cells

The present disclosure also provides, in another embodiment, genes encoding PTD-Smad7 and biologically active fragments and derivatives thereof (including but not limited to, PTD-Smad7(203-426), PTD-Smad7(259-426), and PTD-Smad7(203-217). In addition to the wild-type human SMAD7 gene (GenBank Accession No. AH011391.2), as well as various codon-optimized genes, it should be clear that the present technology is not limited to the specific nucleic acids disclosed herein. As discussed below, a “Smad7 gene” may contain a variety of different bases and yet still produce a corresponding polypeptide that is functionally indistinguishable from, and in some cases structurally identical to, the human gene disclosed herein.

1. Nucleic Acids Encoding Smad7

Nucleic acids according to the present technology may represent an entire Smad7 gene, a fusion with, for example, a protein transduction domain (e.g. Tat), a truncated portion, and/or a fragment of Smad7 (including but not limited to, PTD-Smad7(203-426), PTD-Smad7(259-426), and PTD-Smad7(203-217) that expresses a polypeptide with one or more activity associated with Smad7 such as but not limited to increasing proliferation, reducing or inhibiting cell death, reducing excessive inflammation, preventing DNA damage, and/or increasing cell migration, as well as treating or preventing one or more disease or disorders in which such treatment would be helpful as further discussed herein. Such activities can be assessed using one or more assays including, but not limited to, the ability to block phosphorylation of Smad2 and/or nuclear translocation of the NF-κB p50 subunit, increase cell proliferation, reduce apoptosis and/or radiation-induced DNA damage, reduce inflammation and/or angiogenesis, promote healing in oral mucositis, surgical wounds, diabetes wounds, and/or wounds associated with chronic inflammation in mice. The nucleic acid may be derived from genomic DNA, i.e., cloned directly from the genome of a particular organism. In particular embodiments, however, the nucleic acid would comprise complementary DNA (cDNA). Also provided is a cDNA plus a natural intron or an intron derived from another gene; such engineered molecules are sometime referred to as “mini-genes.” At a minimum, these and other nucleic acids of the present technology may be used as molecular weight standards in, for example, gel electrophoresis.

The term “cDNA” is intended to refer to DNA prepared using messenger RNA (mRNA) as template. The advantage of using a cDNA, as opposed to genomic DNA or DNA polymerized from a genomic, non- or partially-processed RNA template, is that the cDNA primarily contains coding sequences of the corresponding protein. There may be times when the full or partial genomic sequence is preferred, such as where the non-coding regions are required for optimal expression or where non-coding regions such as introns are to be targeted in an antisense strategy.

As used in this application, the term “a nucleic acid encoding a Smad7” may refer to a nucleic acid molecule that has been isolated free of total cellular nucleic acid and/or may refer to a cDNA encoding a Smad7 polypeptide and biologically active fragments and derivatives thereof (including but not limited to, PTD-Smad7(203-426), PTD-Smad7(259-426), and PTD-Smad7(203-217). As used herein, the term “isolated free of total cellular nucleic acid” means that the nucleic acid molecule is about or at least about 75% pure, 80% pure, 85% pure, 90% pure, 95% pure, 96% pure, 97% pure, 98% pure, 99% pure, or 100% pure of other cellular nucleic acid molecules as determined using standard biochemical techniques, such as but not limited to agarose gel electrophoresis. As used herein, the term “isolated free of total cellular protein” means that the protein molecule is about or at least about 75% pure, 80% pure, 85% pure, 90% pure, 95% pure, 96% pure, 97% pure, 98% pure, 99% pure, or 100% pure of other cellular nucleic acid molecules as determined using standard biochemical techniques, such as a western blot. In exemplary embodiments, the present technology concerns a nucleic acid sequence essentially as set forth in, and/or including any one of the nucleotide sequences set forth in SEQ ID NOs: 57, 59, 61, 63, 65, 67, 69, 71, 73, 75, 79, 81, 83, 85, 87, 89, 92, 93, 95, 97, 98, 99, 101, 102, 103, 104, or 105.

An isolated nucleic acid molecule may be produced using recombinant DNA technology (e.g., polymerase chain reaction (PCR) amplification, cloning) or chemical synthesis. Isolated nucleic acid molecules include natural nucleic acid molecules and homologues thereof, including, but not limited to, natural allelic variants and modified nucleic acid molecules in which nucleotides have been inserted, deleted, substituted, and/or inverted in such a manner that such modifications provide the desired effect (e.g., production of Smad7 protein in non-human expression systems).

The term “essentially as set forth in one or more nucleic acid sequence means that the nucleic acid sequence substantially corresponds to at least a portion, and in some cases the entirety, of the one or more nucleic acid sequence (e.g., SEQ ID NOs: 57, 59, 61, 63, 65, 67, 69, 71, 73, 75, 79, 81, 83, 85, 87, 89, 92, 93, 95, 97, 98, 99, 101, 102, 103, 104, and 105). In some embodiments, sequences that substantially correspond to at least a portion of a nucleic acid sequence, may correspond to about, or at least about 50 nucleic acids, 75 nucleic acids, 150 nucleic acids, 200 nucleic acids, 250 nucleic acids, 300 nucleic acids, 350 nucleic acids, 400 nucleic acids, 450 nucleic acids, 500 nucleic acids, 550 nucleic acids, 600 nucleic acids, 650 nucleic acids, 700 nucleic acids, 750 nucleic acids, 800 nucleic acids, 900 nucleic acids, 1000 nucleic acids, 1100 nucleic acids, 1200 nucleic acids, or 1250 nucleic acids of one or more of the sequences described herein. In some embodiments, sequences that substantially correspond to at least a portion of a nucleic acid sequence, may correspond to about a range of about 50-1250 nucleic acids, 75-1250 nucleic acids, 150-1250 nucleic acids, 200-1250 nucleic acids, 250-1250 nucleic acids, 300-1250 nucleic acids, 350-1250 nucleic acids, 400-1250 nucleic acids, 450-1250 nucleic acids, 500-1250 nucleic acids, 550-1250 nucleic acids, 600-1250 nucleic acids, 650-1250 nucleic acids, 700-1250 nucleic acids, 750-1250 nucleic acids, 800-1250 nucleic acids, 900-1250 nucleic acids, 1000-1250 nucleic acids, 1100-1250 nucleic acids, 1200-1250 nucleic acids, at least about 50-75 nucleic acids, 75-150 nucleic acids, 75-200 nucleic acids, 75-250 nucleic acids, 75-300 nucleic acids, 75-350 nucleic acids, 75-400 nucleic acids, 75-450 nucleic acids, 75-500 nucleic acids, 75-550 nucleic acids, 75-600 nucleic acids, 75-650 nucleic acids, 75-700 nucleic acids, 75-750 nucleic acids, 75-800 nucleic acids, 75-900 nucleic acids, 75-1000 nucleic acids, 75-1100 nucleic acids, 75-1200 nucleic acids, or 75-1250 nucleic acids or 1250 nucleic acids of one or more of the sequences described herein.

In some embodiments, sequences that substantially correspond to at least a portion of a nucleic acid sequence include identical sequences to that portion of the nucleic acid sequence. In some embodiments, sequences that substantially correspond to at least a portion of a nucleic acid sequence or the entirety of a nucleic acid sequence may include one or more functionally equivalent codons. The term “functionally equivalent codon” is used herein to refer to one or more codons that encode the same amino acid, such as the six codons for arginine or serine, and in some embodiments, refers to codons that encode biologically equivalent amino acids, as discussed in the following pages. The term “biologically equivalent” amino acid is used herein to refer to one or more amino acids that when changed from the amino acid present in the amino acid sequence of human Smad7 wild-type protein, do not change one or more (or in some embodiments any) of the biological activities of Smad7 described herein, such as but not limited to, increasing proliferation, reducing or inhibiting cell death, reducing excessive inflammation, preventing DNA damage, and/or increasing cell migration, as well as treating or preventing one or more disease or disorders in which such treatment would be helpful as further discussed herein. Such diseases and/or disorders may include but are not limited to acute (e.g., through surgery, combat, trauma) and chronic wounds (e.g., ulcers, such as diabetic, pressure, venous), scarring (including keloid and hypertrophic scarring), fibrosis including lung fibrosis and radiation-induced lung fibrosis in lung cancer patients, and aberrant healing, mucositis (e.g., oral and/or gastro-intestinal, and cancer treatment-induced oral mucositis), stomatitis including recurrent aphthous stomatitis (Canker sore), proctitis, autoimmune disease (e.g., psoriasis, arthritis), radiation-induced dermatitis (radiodermatitis or RT dermatitis), atopic dermatitis, contact dermatitis, allergic dermatitis, Interstitial lung fibrosis (ILF) Radiation Pneumonitis leading to Pulmonary Fibrosis (e.g. due to cancer treating radiation).

In some embodiments, allowing for the degeneracy of the genetic code, sequences that have about or at least about 60%, 70%, 80%, 90%, 91%, 92%, 93%, 94%, 95%, 96%, 97%, 98%, and/or 99% of nucleotides that are identical to the nucleotides of any one of the codon-optimized nucleic acid sequences (e.g., SEQ ID NOs: 57, 59, 90, 92, 93, 95, 98, 99, 101, 102, 103, 104, and 105) may be considered substantially corresponding nucleic acid sequences. Sequences that are essentially the same as those set forth in any one of the nucleic acid sequences (e.g., SEQ ID NOs:) also may be functionally defined as sequences that are capable of hybridizing to a nucleic acid segment containing the complement of SEQ ID NOs: 57, 59, 61, 63, 65, 67, 69, 71, 73, 75, 79, 81, 83, 85, 87, 89, 92, 93, 95, 97, 98, 99, 101, 102, 103, 104, or 105, under various standard conditions.

For applications requiring high selectivity, one will typically desire to employ relatively high stringency conditions to form the hybrids. For example, relatively low salt and/or high temperature conditions, such as provided by about 0.02 M to about 0.10 M NaCl at temperatures of about 50° C. to about 70° C. Such high stringency conditions tolerate little, if any, mismatch between the probe or primers and the template or target strand and would be particularly suitable for isolating specific genes or for detecting specific mRNA transcripts. It is generally appreciated that conditions can be rendered more stringent by the addition of increasing amounts of formamide.

For certain applications, it is appreciated that lower stringency conditions are preferred. Under these conditions, hybridization may occur even though the sequences of the hybridizing strands are not perfectly complementary, but are mismatched at one or more positions. Conditions may be rendered less stringent by increasing salt concentration and/or decreasing temperature. For example, a medium stringency condition could be provided by about 0.1 to 0.25 M NaCl at temperatures of about 37° C. to about 55° C., while a low stringency condition could be provided by about 0.15 M to about 0.9 M salt, at temperatures ranging from about 20° C. to about 55° C. Hybridization conditions can be readily manipulated depending on the desired results.

In other embodiments, hybridization may be achieved under conditions of, for example, 50 mM Tris-HCl (pH 8.3), 75 mM KCl, 3 mM MgCl₂, 1.0 mM dithiothreitol, at temperatures between approximately 20° C. to about 37° C. Other hybridization conditions utilized could include approximately 10 mM Tris-HCl (pH 8.3), 50 mM KCl, 1.5 mM MgCl₂, at temperatures ranging from approximately 40° C. to about 72° C.

To determine the percent homology of two amino acid sequences or of two nucleic acids, the sequences are aligned for optimal comparison purposes (e.g., gaps are introduced in the sequence of a first amino acid or nucleic acid sequence for optimal alignment with a second amino acid or nucleic acid sequence). The amino acid residues or nucleotides at corresponding amino acid positions or nucleotide positions can then be compared. When a position in the first sequence is occupied by the same amino acid residue or nucleotide as the corresponding position in the second sequence, then the molecules are identical at that position. The percent homology between the two sequences is a function of the number of identical positions shared by the sequences (% identity=# of identical positions/total # of positions (e.g., overlapping positions×100). In some embodiments, the two sequences are the same length.

To determine percent homology between two sequences, the algorithm of Karlin and Altschul (1990) Proc. Natl. Acad. Sci. USA 87:2264-2268, modified as in Karlin and Altschul (1993) Proc. Natl. Acad. Sci. USA 90:5873-5877 can be used. Such an algorithm is incorporated into the NBLAST and XBLAST programs of Altschul et al (1990) J. Mol Biol. 215:403-410. BLAST nucleotide searches are performed with the NBLAST program, score=100, wordlength=12 to obtain nucleotide sequences homologous to a nucleic acid molecules described or disclose herein. BLAST protein searches are performed with the XBLAST program, score=50, wordlength=3. To obtain gapped alignments for comparison purposes, Gapped BLAST may be utilized as described in Altschul et al. (1997) Nucleic Acids Res. 25:3389-3402. When utilizing BLAST and Gapped BLAST programs, the default parameters of the respective programs (for example,)(BLAST and NBLAST) are used. See the website of the National Center for Biotechnology Information for further details (on the World Wide Web at ncbi.nlm.nih.gov). Proteins suitable for use in the methods described herein also includes proteins having between 1 to 15 amino acid changes, for example, 1, 2, 3, 4, 5, 6, 7, 8, 9, 10, 11, 12, 13, 14, or 15 amino acid substitutions, deletions, or additions, compared to the amino acid sequence of any protein described herein. In other embodiments, the altered amino acid sequence is at least 75% identical, for example, 77%, 80%, 82%, 85%, 88%, 90%, 92%, 95%, 97%, 98%, 99%, or 100% identical to the amino acid sequence of any protein inhibitor described herein. Such sequence-variant proteins are suitable for the methods described herein as long as the altered amino acid sequence retains sufficient biological activity to be functional in the compositions and methods described herein. In certain instances, conservative amino acid substitutions are utilized. Illustrative conservative substitution among amino acids are within each of the following groups: (1) glycine, alanine, valine, leucine, and isoleucine, (2) phenylalanine, tyrosine, and tryptophan, (3) serine and threonine, (4) aspartate and glutamate, (5) glutamine and asparagine, and (6) lysine, arginine and histidine. The BLOSUM62 table is an amino acid substitution matrix derived from about 2,000 local multiple alignments of protein sequence segments, representing highly conserved regions of more than 500 groups of related proteins (Henikoff et al. (1992), Proc. Natl Acad. Sci. USA, 89:10915-10919). The BLOSUM62 substitution frequencies can be used to define conservative amino acid substitutions that, in some embodiments, are introduced into the amino acid sequences described or disclosed herein. Although it is possible to design amino acid substitutions based solely upon chemical properties (as discussed above), the language “conservative amino acid substitution” preferably refers to a substitution represented by a BLOSUM62 value of greater than −1. For example, an amino acid substitution is conservative if the substitution is characterized by a BLOSUM62 value of 0, 1, 2, or 3. According to this system, preferred conservative amino acid substitutions are characterized by a BLOSUM62 value of at least 1 (e.g., 1, 2 or 3), while more preferred conservative amino acid substitutions are characterized by a BLOSUM62 value of at least 2 (e.g., 2 or 3).

The DNA segments of the present technology include those encoding biologically functional equivalent Smad7 proteins and peptides (including but not limited to, PTD-Smad7(203-426), PTD-Smad7(259-426), and PTD-Smad7(203-217), as described above. Such sequences may arise as a consequence of codon redundancy and amino acid functional equivalency that are known to occur naturally within nucleic acid sequences and the proteins thus encoded. Alternatively, functionally equivalent proteins or peptides may be created via the application of recombinant DNA technology, in which changes in the protein structure may be engineered, based on considerations of the properties of the amino acids being exchanged. Changes designed by man may be introduced through the application of site-directed mutagenesis techniques or may be introduced randomly and screened later for the desired function, as described elsewhere.

As described in greater detail below, the Smad7 nucleic acid sequence (and those fragments encoding biologically active fragments and derivatives thereof (including but not limited to, PTD-Smad7(203-426), PTD-Smad7(259-426), and PTD-Smad7(203-217) has been optimized for expression in alternative host organisms (e.g., non-human). Although as described above, the genetic code is degenerate, so frequently one amino acid may be coded for by two or more nucleotide codons. Thus, multiple nucleic acid sequences may encode one amino acid sequence. Although this creates identical proteins, the nucleic acids themselves are distinct, and can have distinct properties. As described herein, one aspect of the choice of codon usage can be (but is not limited to) the ability to express a protein in non-native cells (e.g., a human protein in bacteria or yeast), or the level of expression in such cells. In order to obtain enough protein for purification, testing, and use in in vitro assays, in animal models, and eventually in clinical development, efficient protein expression in non-human systems is needed.

A series of 23 arginine amino acids in the human Smad7 protein sequence coded for by one or more of AGG (1.7% codon utilization; 9 residues), AGA (2.8% codon utilization; 2 residues), CGA (3.5% codon utilization; 4 residues), or CGG (5.4% codon utilization; 8 residues) has been identified, and it has been determined that in order to have efficient protein expression from non-human sources, such as, but not limited to, bacteria and/or yeast that one or more, and potentially all the arginine codons should be modified to CGT (20.6% codon utilization). Therefore, in some embodiments, the Smad7 codon-optimized nucleic acid sequence includes at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, or 23 codons for arginine that have been changed to CGT. In some embodiments, the Smad7 codon-optimized nucleic acid sequence includes one or more or all of the arginine codons at nucleic acid sequence positions 7-9, 43-45, 169-171, 403-405, 490-492, 526-528, 526-528, 823-825, 1057-1059, 16-18, 136-138, 199-201, 598-600, 31-33, 112-114, 316-318, 772-774, 940-942, 973-975, 1135-1137, 1276-1278, 637-639, or 814-816 be changed to CGT.

A series of 33 serine residues in the human Smad7 protein sequence coded for by TCC or TCG (9%) has been identified, and it has been determined that it may be beneficial to efficient protein expression and purification from non-human sources, such as, but not limited to, bacteria and/or yeast, that one or more, and potentially all the serine codons be modified to AGC (15% codon utilization). Therefore, in some embodiments, the Smad7 codon-optimized nucleic acid sequence includes at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32 or 33 codons for serine that have been changed to (AGC). In some embodiments, the Smad7 codon-optimized nucleic acid sequence includes one or more or all of the serine codons at nucleic acid sequence positions 19-21, 46-48, 133-135, 292-294, 349-351, 451-453, 454-456, 460-462, 511-513, 514-516, 544-546, 595-597, 616-618, 634-636, 691-693, 694-696, 739-741, 745-747, 775-777, 847-849, 907-909, 919-921, 943-945, 1006-1008, 1009-1101, 1030-1032, 1054-1056, 1093-1095, 1126-1128, 1192-1194, 1237-1239, 1240-1242, 1273-1275. Of these, 23 codons (19-21, 292-294, 349-351, 451-453, 454-456, 460-462, 511-513, 514-516, 544-546, 616-618, 634-636, 691-693, 694-696, 739-741, 745-747, 775-777, 847-849, 907-909, 919-921, 1009-1101, 1030-1032, 1054-1056, 1093-1095) can be changed without introducing potential alternative open reading frames.

A series of 12 histidine residues in the human Smad7 protein sequence coded for by CAC (9.6% codon usage) has also been identified, and it has been determined that it may be beneficial to efficient protein expression and purification from non-human sources, such as but not limited to bacteria and/or yeast, that one or more, and potentially all the serine codons be modified to CAT (optionally to 12.6% usage). Therefore, in some embodiments, the Smad7 codon-optimized nucleic acid sequence includes at least 1, at least 2, at least 3, at least 4, at least 5, at least 6, at least 7, at least 8, at least 9, at least 10, at least 11, or 12 codons for histidine that have been changed to (CAT). In some embodiments, the Smad7 codon-optimized nucleic acid sequence includes one or more or all of the serine codons at nucleic acid sequence positions 142-144, 214-216, 217-219, 220-222, 226-228, 289-291, 589-591, 778-780, 1072-1074, 1147-1149. Of these, 4 codons (nucleotides 217-219, 220-222, 589-591, 778-780) can be changed without introducing potential alternative open reading frames.

In some embodiments, one or more codon-optimized nucleic acids may include one or more of at least one and any integer up to 22 of its arginine codons modified to CGT, at least one and any integer up to 28 of its serine codons (optionally that are able to be modified with introducing open reading frames) modified to AGC, or at least one and any integer up to 12 of its histidine codons (optionally that are able to be modified with introducing open reading frames) modified to CAT. In some embodiments, one or more codon-optimized nucleic acid may include at least one and any integer up to 22 of its arginine codons modified to CGT, at least one and any integer up to 28 of its serine codons (optionally that are able to be modified with introducing open reading frames) modified to AGC, and at least one and any integer up to 12 (optionally that are able to be modified with introducing open reading frames) of its histidine codons modified to CAT. In some embodiments, one or more codon-optimized nucleic acid may include 22 of its arginine codons modified to CGT, 28 of its serine codons (optionally that are able to be modified with introducing open reading frames) modified to AGC, and 12 of its histidine codons (optionally that are able to be modified with introducing open reading frames) modified to CAT. In some embodiments, one or more codon-optimized nucleic acid may also have a nucleotide substitution in the codon for Met216 (ATG), to form the codon for Leu216 (CTG).

In some embodiments, one or more codon-optimized nucleic acids may have about 65% to 75%, about 65% to 68%, about 68% to 75%, or about 68% to 71% homology to human Smad7 wild-type cDNA (SEQ ID NO: 2). In some embodiments, one or more codon-optimized nucleic acid may have about 65%, 66%, 67%, 68%, 69%, 70%, 71%, 72%, 73%, 74%, or 75%, homology to human Smad7 wild-type cDNA (SEQ ID NO: 2). In some embodiments, one or more codon-optimized nucleic acid may also have a nucleotide substitution in the codon for Met216 (ATG), to form the codon for Leu216 (CTG).

A methionine codon (Met216; ATG) that has the potential for being perceived by translation machinery (e.g., such as but not limited bacteria or yeast) as an alternative open reading frame has been identified. Although not intending to be bound by theory, it is believed that the presence of the second potential open reading frame may decrease expression of the Smad7 protein. In some embodiments, one or more Smad7 nucleic acid sequences are modified at nucleotide position (646-648) to encode a human Smad7 protein where Met216 (ATG) is modified to Leu216 (CTG).

It has also been discovered that various truncated forms and fragments of Smad7 (including but not limited to, PTD-Smad7(203-426), PTD-Smad7(259-426), and PTD-Smad7(203-217) protein retain one or more of the activities of full-length human Smad7, such as, but not limited to, increasing proliferation, reducing or inhibiting cell death, reducing excessive inflammation, preventing DNA damage, and/or increasing cell migration, as well as treating or preventing one or more disease or disorders in which such treatment would be helpful as further discussed herein. Such diseases and/or disorders may include but are not limited to acute (e.g., through surgery, combat, trauma, diabetes) and chronic wounds (e.g., ulcers, such as diabetic, pressure, venous), scarring (including keloid and hypertrophic scarring), fibrosis including lung fibrosis and radiation-induced lung fibrosis in lung cancer patients, and aberrant healing, mucositis (e.g., oral and/or gastro-intestinal, and cancer treatment-induced oral mucositis), stomatitis including recurrent aphthous stomatitis (Canker sore), proctitis, autoimmune disease (e.g., psoriasis, arthritis), radiation-induced dermatitis (radiodermatitis or RT dermatitis), atopic dermatitis, contact dermatitis, allergic dermatitis, Interstitial lung fibrosis (ILF) Radiation Pneumonitis leading to Pulmonary Fibrosis (e.g. due to cancer treating radiation). Such activities can be assessed using one or more assays including, but not limited to, the ability to block phosphorylation of Smad2 and/or nuclear translocation of the NF-κB p50 subunit, increase cell proliferation, reduce apoptosis and/or radiation-induced DNA damage, reduce inflammation and/or angiogenesis, promote healing in oral mucositis, surgical wounds, diabetes wounds, and/or wounds associated with chronic inflammation in mice.

Further, in some embodiments, various truncated forms and fragments of Smad7 protein retain only a subset of the one or more of the activities of full-length human Smad7. For example, the C-terminal MH2 domain of Smad7 may primarily mediate the anti-inflammatory effect of Smad7. Smad7 peptides having this anti-inflammatory function may be sufficient and optionally an improvement for treating chronic inflammation associated conditions, such as but not limited to, oral mucositis, stomatitis, arthritis, and psoriasis, among others. The N-terminal MH1 domain may primarily mediate cell migration and/or blocking TGF-β-induced growth arrest and/or fibrotic response. Smad7 peptides having this cell migration and proliferation function may be sufficient, and optionally an improvement, for enhancing healing that is not associated with excessive inflammation. Types of wounds that may benefit from this form of treatment include, but are not limited to, surgical wounds, fibrotic scarring, and diabetes wounds, defective healing and/or scarring among others.

In some embodiments, nucleic acid molecules (optionally codon-optimized nucleic acid molecules as described above and herein) encode fragments or truncated forms of Smad7 protein (optionally including Leu216). In some embodiments, these fragments and/or truncated forms of Smad7 protein retain one or more or all of the activities of full-length human Smad7 protein. In some embodiments, such truncated nucleic acid sequences encode the N-terminal portion of the Smad7 protein. In some embodiments, such truncated nucleic acid sequences encode the C-terminal portion of the Smad7 protein. In some embodiments, such fragments of the nucleic acid sequences (nucleotide positions 610-1278) encode amino acids 203-426 of the human Smad7 protein.

The term “truncated” as used herein in reference to nucleic acid molecules refers to a molecule that contains nucleotide sequences encoding the natural N-terminus of a corresponding protein (with or without a cleaved leader sequence), but lacks one or more nucleotides starting from the C-terminus-encoding portion of the molecule, or a molecule that contains nucleotide sequences encoding the natural C-terminus of a corresponding protein (with or without a cleaved leader sequence), but lacks one or more nucleotides starting from the N-terminus-encoding portion of the molecule. In some embodiments, molecules lacking nucleotides encoding at least about 25, at least about 50, at least about 75, at least about 100, at least about 125, at least about 150, at least about 200, at least about 250, at least about 300, or at least about 350, or at least about 400 amino acids from one or the other terminus are specifically provided. Similarly, the term “truncated” may also be used in reference to protein molecules encoded by truncated nucleic acid molecules. In some embodiments, a “truncated” molecule is biologically active, having (or encoding a polypeptide having) one or more of the Smad7 activities described herein (including but not limited to, PTD-Smad7(203-426) and PTD-Smad7(259-426).

The term “fragment” as used herein in reference to nucleic acid molecules refers to a molecule containing contiguous residues of a full-length sequence but lacking some 5′ and/or 3′ sequences of the full-length sequence (including but not limited to, PTD-Smad7(203-217). In some embodiments, a “fragment” includes a portion of one or more of the full-length sequences described herein. In some embodiments, the “fragment” does not include sequences encoding either the N-terminal or the C-terminal, but only internal fragments. In some embodiments, a “fragment” encodes a polypeptide that is biologically active, having one or more of the Smad7 activities described herein. In some embodiments, nucleic acid fragments may encode proteins having at least about 15, 20, 25, 30, 35, 40, 45, 50, 55, 60, 65, 70, 75, 80, 85, 90, 95, 100, 150, 200, 250, 300, 350, 400, 450, 500, 550, 600, 650, 700, 750, 800, 850, 900, 950, 1000, 1050, 1100, 1150 amino acids. Similarly, “fragment” may also be used in reference to protein molecules encoded by Smad7 nucleic acid fragments.

The term “N-terminal portion” as used herein in refers to a fragment of a corresponding protein that contains the protein's N-terminus but lacks all sequences C-terminal to an internal residue.

The term “C-terminal portion” as used herein in refers to a fragment of a corresponding protein that contains the protein's C-terminus but lacks all sequences N-terminal to an internal residue.

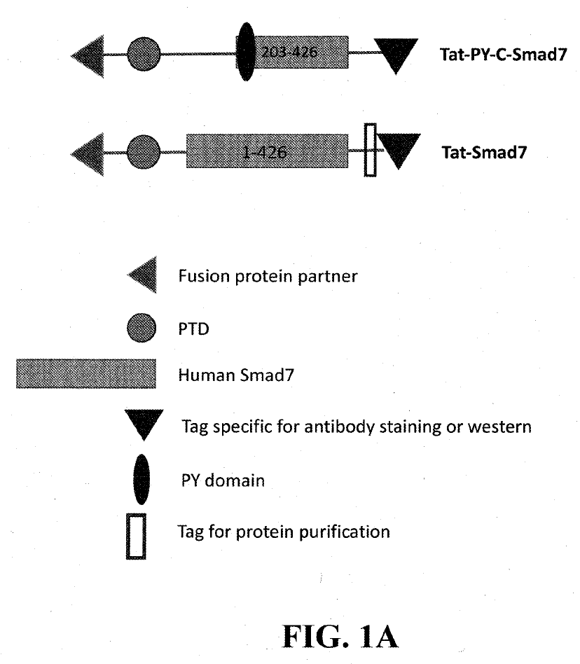

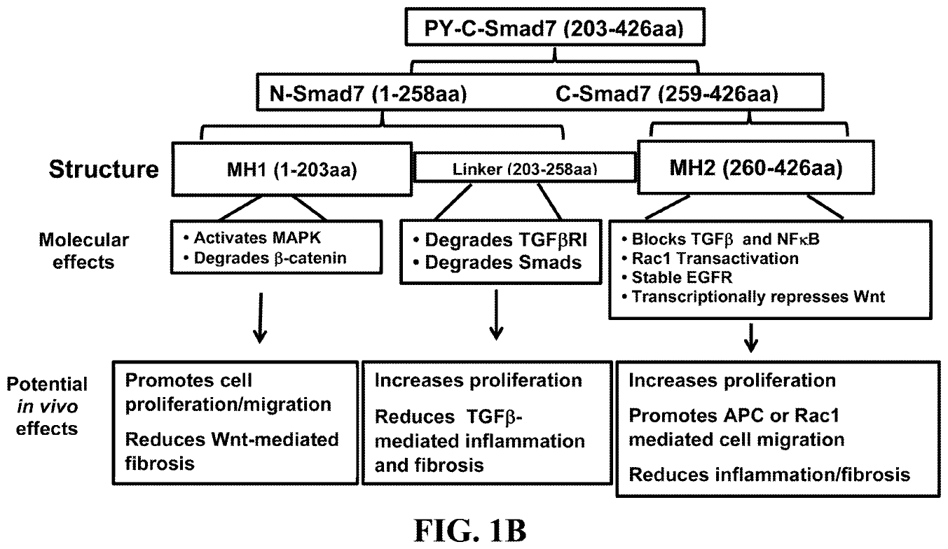

The inventor previously developed a biologic containing human Smad7 fused to a small peptide from the HIV-1 Tat protein that rapidly penetrates cells upon contact (Han G, Bian L, Li F, Cotrim A, Wang D, Lu J, et al. Preventive and therapeutic effects of Smad7 on radiation-induced oral mucositis. Nat Med. 2013; 19(4):421-8; U.S. patent application Ser. No. 14/773,167, Publication No. US 2016/0039894, which is incorporated herein in its entirety). The inventor has since made several Tat-Smad7 truncated variants (U.S. patent application Ser. No. 15/208,424; U.S. Pub. No. 2017/0000851, which is incorporated herein in its entirety), and shown that they generally retain some, but not all, of the activities of the full-length Tat-Smad7 construct. In addition, the activity is typically lower than that of the full-length Tat-Smad7 construct as might be expected, presumably because of changes in tertiary structure, degradation properties, or lack of interplay with other portions of the protein, such as sequences providing the ability to traffic between the nucleus and the cytoplasm. FIG. 1B summarizes potential molecular mechanisms of the multifunctional nature of Tat-PY-C-Smad7.

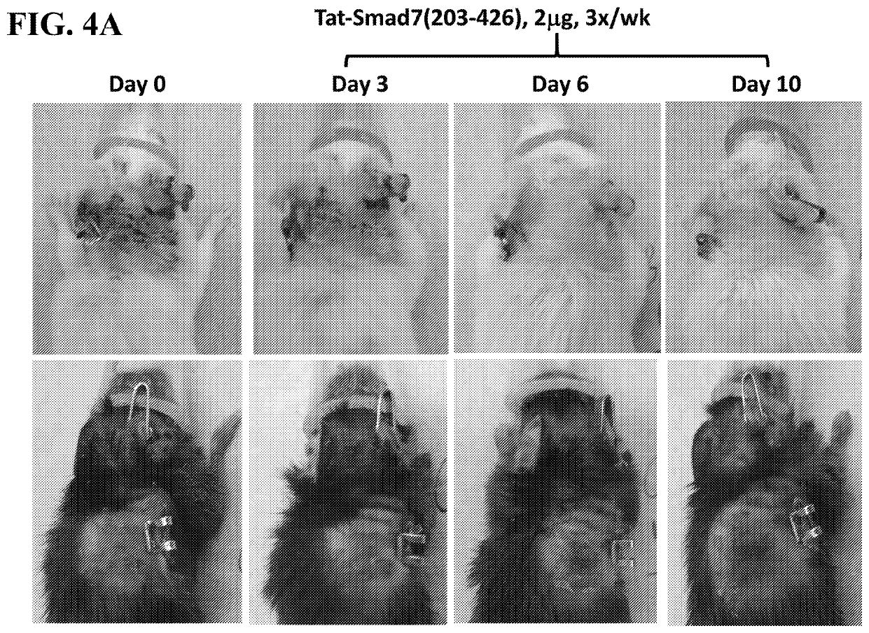

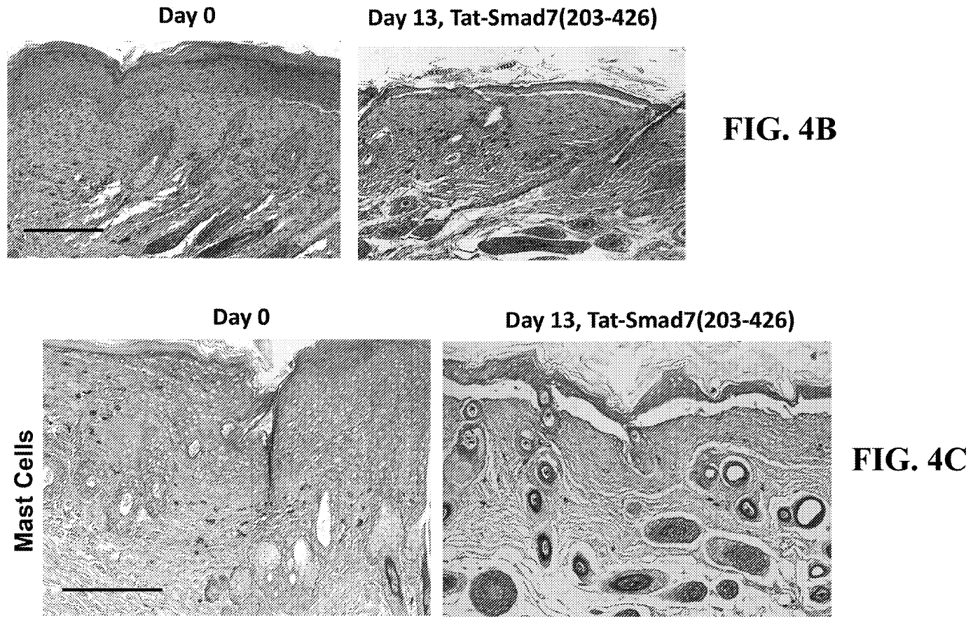

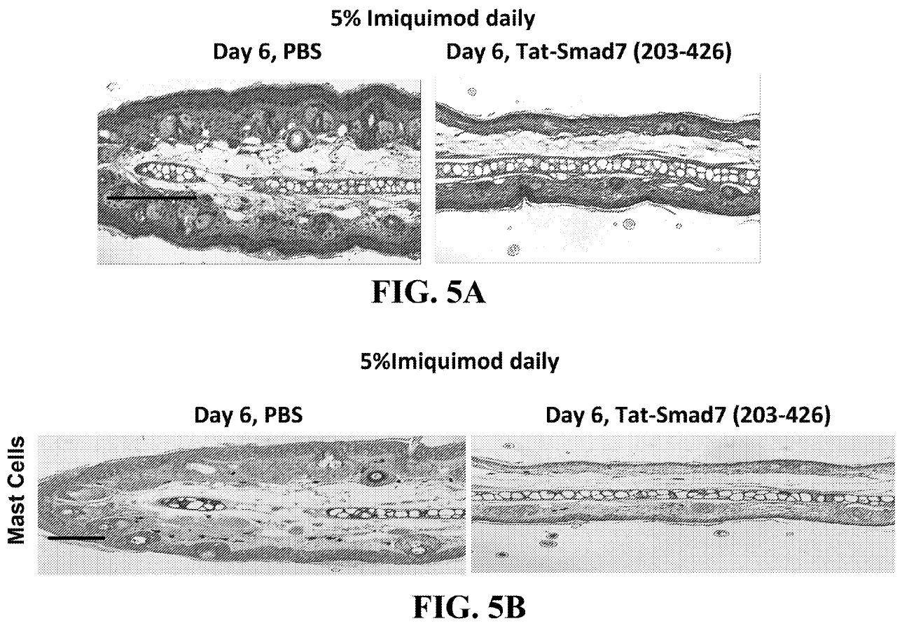

Even more surprisingly, the inventor has discovered that Tat-PY-C-Smad7 containing amino acids 203-426aa of human Smad7, has substantially higher biological acitivities than the Tat-Smad7 fragment constructs described in US 2016/0039894. This new construct, Tat-PY-C-Smad7, also unexpectedly has activities similar to, or better than, full length Tat-Smad7 in multiple medical indications. For example, in a recent publication (Li et al., Journal of Investigative Dermatology, doi: 10.1016/j.jid.2018.10.031. [Epub ahead of print] PMID: 30423327), the study led by the inventor shows that it takes 18 days for full length Tat-Smad7 to show gross alleviation of skin inflammation in K5.TGFβ1 skin and 28 days to completely reverse skin inflammation in this model. By comparison, FIG. 4 in this application shows that it only took 6 days for Tat-PY-C-Smad7 (203-426) to alleviate skin inflammation grossly and 13 days to completely reverse skin inflammation in the same model. This discovery was completely unexpected as none of the inventor's previous data provided any indication that the amino acids 203-426aa of human Smad7 fragment, linked in this fusion construct would have significantly higher, or even significantly different, therapeutic activities compared to the full length human Smad7-Tat fusion construct.

In some embodiments, the PTD is located at the 3′ end of the Smad7 nucleic acid sequence, and in some embodiments the PTD is located at the 5′ end of the Smad7 nucleic acid sequence. In some embodiments, there is a linker sequence encoding 1, 2, 3, 4, 5, or 6 amino acids that connects the PTD and the Smad7 nucleic acid sequence.

In some embodiments, the PTD nucleic acid sequence is a Tat nucleic acid sequence. ggccgtaaaaaacgccgtcaacgccgccgt (SEQ ID NO: 52) encoding GRKKRRQRRR (SEQ ID NO: 6), tatggccgtaaaaaacgccgtcaacgccgccgt (SEQ ID NO: 53) encoding YGRKKRRQRRR (SEQ ID NO: 7), or ggccgtaaaaaacgccgtcaa (SEQ ID NO: 54) encoding GRKKRRQ (SEQ ID NO: 55). Alternatively or additionally, the PTD nucleic acid sequence may be a PEP peptide sequence (KETWWETWWTEWSQPKKKRKV (SEQ ID NO: 29)).

In some embodiments, the nucleic acid sequence further includes a nucleotide sequence encoding one or more of an epitope tag or a purification tag. In some embodiments, the epitope tag is V5. In some embodiments, the purification tag is one or more of glutathione-S-Transferase (GST) or 6-histidine (H6) (SEQ ID NO: 56).

The term “epitope tag” as used herein in reference to nucleic acid molecules refers to nucleotides encoding peptide sequences that are recognized and bound by the variable region of an antibody or fragment. In some embodiments, the epitope tag is not part of the native protein. In some embodiments, the epitope tag is removable. In some embodiments, the epitope tag is not intrinsic to the protein's native biological activity. Examples of epitope tags include, but are not limited to V5.

The term “purification tag” as used herein in reference to nucleic acid molecules refers to nucleotides encoding peptide sequences that facilitate the purification of the protein, but are generally not necessary for the protein's biological activity. In some embodiments, purification tags may be removed following protein purification. Examples of purification tags include, but are not limited to GST and H-6 (SEQ ID NO: 56).

2. Vectors for Cloning, Gene Transfer and Expression

Within certain embodiments, expression vectors are employed to express the Smad7 polypeptide product, which can then be purified for various uses. In other embodiments, the expression vectors are used in gene therapy. Expression requires that appropriate signals be provided in the vectors, and which include various regulatory elements, such as enhancers/promoters from both viral and mammalian sources that drive expression of the genes of interest in host cells. Elements designed to optimize messenger RNA stability and translatability in host cells also are defined. The conditions for the use of a number of dominant drug selection markers for establishing permanent, stable cell clones expressing the products are also provided, as is an element that links expression of the drug selection markers to expression of the polypeptide.

Throughout this application, the term “expression construct” is meant to include any type of genetic construct containing a nucleic acid coding for a gene product in which part or all of the nucleic acid encoding sequence is capable of being transcribed. The transcript may be translated into a protein, but it need not be. In certain embodiments, expression includes both transcription of a gene and translation of mRNA into a gene product. In other embodiments, expression only includes transcription of the nucleic acid encoding a gene of interest.

The term “vector” is used to refer to a carrier nucleic acid molecule into which a nucleic acid sequence can be inserted for introduction into a cell where it can be replicated. A nucleic acid sequence can be “exogenous,” which means that it is foreign to the cell into which the vector is being introduced or that the sequence is homologous to a sequence in the cell but in a position within the host cell nucleic acid in which the sequence is ordinarily not found. Vectors include plasmids, cosmids, viruses (bacteriophage, animal viruses, and plant viruses), and artificial chromosomes (e.g., YACs). One of skill in the art would be well-equipped to construct a vector through standard recombinant techniques, which are described, e.g., in Sambrook, et al., Molecular Cloning (Cold Spring Harbor Lab Press, 1989), and Ausubel, et al., Current Protocols in Molecular Biology (Wiley, 1994), both incorporated herein by reference.

The term “expression vector” refers to a vector containing a nucleic acid sequence coding for at least part of a gene product capable of being transcribed. In some cases, RNA molecules are then translated into a protein, polypeptide, or peptide. In other cases, these sequences are not translated, for example, in the production of antisense molecules or ribozymes. Expression vectors can contain a variety of “control sequences,” which refer to nucleic acid sequences necessary for the transcription and possibly translation of an operably linked coding sequence in a particular host organism, including promoters and enhancers. In addition to control sequences that govern transcription and translation, vectors and expression vectors may contain nucleic acid sequences that serve other functions, such as transcription termination signals and poly-adenylation sites.

The capacity of certain viral vectors to efficiently infect or enter cells, to integrate into a host cell genome and stably express viral genes, have led to the development and application of a number of different viral vector systems. Robbins, et al., Pharmacol. Ther. 80:35-47 (1998). Viral systems are currently used as vectors for ex vivo and in vivo gene transfer. For example, adenovirus, herpes-simplex virus, lentiviruses, retrovirus and adeno-associated virus vectors are being evaluated currently for treatment of diseases such as cancer, cystic fibrosis, Gaucher disease, renal disease and arthritis. Robbins, et al., Pharmacol. Ther. 80:35-47 (1998); Imai, et al., Nephrologie 19:379-402 (1998); U.S. Pat. No. 5,670,488. The various viral vectors present specific advantages and disadvantages, depending on the particular gene-therapeutic application.

Suitable non-viral methods for nucleic acid delivery for transformation of an organelle, a cell, a tissue or an organism for use with the present technology are believed to include virtually any method by which a nucleic acid (e.g., DNA) can be introduced into an organelle, a cell, a tissue or an organism, as described herein or as would be known to one of ordinary skill in the art. Such methods include, but are not limited to, direct delivery of DNA such as by injection (U.S. Pat. Nos. 5,994,624, 5,981,274, 5,945,100, 5,780,448, 5,736,524, 5,702,932, 5,656,610, 5,589,466 and 5,580,859, each incorporated herein by reference), including microinjection (Harland and Weintraub, 1985; U.S. Pat. No. 5,789,215, incorporated herein by reference); by electroporation (U.S. Pat. No. 5,384,253, incorporated herein by reference); by calcium phosphate precipitation (Graham, et al., Virology 52:456-467 (1973); Chen, et al., Mol. Cell Biol. 7:2745-2752 (1987); Rippe, et al., Mol. Cell Biol. 10:689-695 (1990)); by using DEAE-dextran followed by polyethylene glycol (Gopal, Mol. Cell Biol. 5:1188-1190 (1985)); by direct sonic loading (Fechheimer, et al., PNAS 84:8463-8467 (1987)); by liposome mediated transfection (Nicolau, et al., Biochim. Biophys. Acta 721:185-190 (1982); Fraley, et al., PNAS 76:3348-3352 (1979); Nicolau, et al., Methods Enzymol. 149: 157-176 (1987); Wong, et al., Gene 10:87-94 (1980); Kaneda, et al., J. Biol. Chem. 264:12126-12129 (1989); Kato, et al., J. Biol. Chem. 266:3361-3364 (1991)); by microprojectile bombardment (PCT Application Nos. WO 94/09699 and 95/06128; U.S. Pat. Nos. 5,610,042; 5,322,783, 5,563,055, 5,550,318, 5,538,877 and 5,538,880, and each incorporated herein by reference); by agitation with silicon carbide fibers (Kaeppler, et al., Plant Cell Rep. 9:415-418 (1990); U.S. Pat. Nos. 5,302,523 and 5,464,765, each incorporated herein by reference); or by PEG-mediated transformation of protoplasts (Omirulleh, et al., Plant Mol. Biol. 21:415-428 (1993); U.S. Pat. Nos. 4,684,611 and 4,952,500, each incorporated herein by reference); by desiccation/inhibition-mediated DNA uptake (Potrykus, et al., Mol. Gen. Genet. 199:169-177 (1985)). Through the application of techniques such as these, organelle(s), cell(s), tissue(s) or organism(s) may be stably or transiently transformed.

3. Expression Systems

Numerous expression systems exist that comprise at least a part or all of the compositions discussed above. Prokaryote- and/or eukaryote-based systems can be employed for use with the present technology to produce nucleic acid sequences, or their cognate polypeptides, proteins and peptides. Many such systems are commercially and widely available.

The insect cell/baculovirus system can produce a high level of protein expression of a heterologous nucleic acid segment, such as described in U.S. Pat. Nos. 5,871,986 and 4,879,236, both herein incorporated by reference, and which can be bought, for example, under the name MAXBAC® 2.0 from INVITROGEN® and BACPACK™ BACULOVIRUS EXPRESSION SYSTEM FROM CLONTECH®.

Other examples of expression systems include STRATAGENE®'s COMPLETE CONTROL™ Inducible Mammalian Expression System, which involves a synthetic ecdysone-inducible receptor, or its pET Expression System, an E. coli expression system. Another example of an inducible expression system is available from INVITROGEN®, which carries the T-REX™ (tetracycline-regulated expression) System, an inducible mammalian expression system that uses the full-length CMV promoter. INVITROGEN® also provides a yeast expression system called the Pichia methanolica Expression System, which is designed for high-level production of recombinant proteins in the methylotrophic yeast Pichia methanolica. One of skill in the art would know how to express a vector, such as an expression construct, to produce a nucleic acid sequence or its cognate polypeptide, protein, or peptide.

Primary mammalian cell cultures may be prepared in various ways. In order for the cells to be kept viable while in vitro and in contact with the expression construct, it is necessary to ensure that the cells maintain contact with the correct ratio of oxygen and carbon dioxide and nutrients but are protected from microbial contamination. Cell culture techniques are well documented.

One embodiment of the foregoing involves the use of gene transfer to immortalize cells for the production of proteins. The gene for the protein of interest may be transferred as described above into appropriate host cells followed by culture of cells under the appropriate conditions. The gene for virtually any polypeptide may be employed in this manner. The generation of recombinant expression vectors, and the elements included therein, are discussed above. Alternatively, the protein to be produced may be an endogenous protein normally synthesized by the cell in question.

Examples of useful mammalian host cell lines are Vero and HeLa cells and cell lines of Chinese hamster ovary, W138, BHK, COS-7, 293, HepG2, NIH3T3, RIN and MDCK cells. In addition, a host cell strain may be chosen that modulates the expression of the inserted sequences, or modifies and process the gene product in the manner desired. Such modifications (e.g., glycosylation) and processing (e.g., cleavage) of protein products may be important for the function of the protein. Different host cells have characteristic and specific mechanisms for the post-translational processing and modification of proteins. Appropriate cell lines or host systems can be chosen to insure the correct modification and processing of the foreign protein expressed.

A number of selection systems may be used including, but not limited to, HSV thymidine kinase, hypoxanthine-guanine phosphoribosyltransferase and adenine phosphoribosyltransferase genes, in tk-, hgprt- or aprt-cells, respectively. Also, anti-metabolite resistance can be used as the basis of selection for dhfr, that confers resistance to; gpt, that confers resistance to mycophenolic acid; neo, that confers resistance to the aminoglycoside G418; and hygro, that confers resistance to hygromycin.

As used herein, the terms “cell,” “cell line,” and “cell culture” may be used interchangeably. All of these terms also include their progeny, which are any and all subsequent generations. It is understood that all progeny may not be identical due to deliberate or inadvertent mutations. In the context of expressing a heterologous nucleic acid sequence, “host cell” refers to a prokaryotic or eukaryotic cell, and it includes any transformable organism that is capable of replicating a vector and/or expressing a heterologous gene encoded by a vector. A host cell can, and has been, used as a recipient for vectors. A host cell may be “transfected” or “transformed,” which refers to a process by which exogenous nucleic acid is transferred or introduced into the host cell. A transformed cell includes the primary subject cell and its progeny.

Host cells may be derived from prokaryotes or eukaryotes (e.g., bacteria or yeast), depending upon whether the desired result is replication of the vector or expression of part or all of the vector-encoded nucleic acid sequences. Numerous cell lines and cultures are available for use as a host cell, and they can be obtained through the American Type Culture Collection (ATCC), which is an organization that serves as an archive for living cultures and genetic materials (atcc.org). An appropriate host can be determined by one of skill in the art based on the vector backbone and the desired result. A plasmid or cosmid, for example, can be introduced into a prokaryote host cell for replication of many vectors. Bacterial cells used as host cells for vector replication and/or expression include DH5α, JM109, and KC8, as well as a number of commercially available bacterial hosts such as SURE® Competent Cells and SOLOPACK™ Gold Cells (STRATAGENE®, La Jolla). Alternatively, bacterial cells such as E. coli LE392 could be used as host cells for phage viruses.

Examples of eukaryotic host cells for replication and/or expression of a vector include HeLa, NIH3T3, Jurkat, 293, Cos, CHO, Saos, and PC12. Many host cells from various cell types and organisms are available and would be known to one of skill in the art. Similarly, a viral vector may be used in conjunction with either a eukaryotic or prokaryotic host cell, particularly one that is permissive for replication or expression of the vector.

Some vectors may employ control sequences that allow it to be replicated and/or expressed in both prokaryotic and eukaryotic cells. One of skill in the art would further understand the conditions under which to incubate all of the above described host cells to maintain them and to permit replication of a vector. Also understood and known are techniques and conditions that would allow large-scale production of vectors, as well as production of the nucleic acids encoded by vectors and their cognate polypeptides, proteins, or peptides.

### B. Smad7 Proteins and Protein Fragments

Mothers against decapentaplegic homolog 7 (Smad7) was previously identified as an antagonist of TGF-β signaling by several mechanisms including: (a) blockade of TGF-β receptor-mediated phosphorylation and nuclear translocation of signaling Smads; (b) increased degradation of TGF-β receptors and signaling Smads through specific ubiquitin-proteasome pathways and (c) inhibition of signaling Smads for their binding to Smad binding elements (SBEs). Smad7 also antagonizes other signaling pathways, like the NF-κB pathway.

Smad7 protein is encoded by the SMAD7 gene, discussed above. Like many other TGF-β family members, Smad7 is involved in cell signaling. It is a TGF-β type 1 receptor antagonist. It blocks TGF-β1 and activin associating with the receptor, blocking access to Smad2. It is an inhibitory Smad (I-SMAD) and is enhanced by SMURF2. Smad7 also enhances muscle differentiation.

The terms “polypeptide,” “peptide,” and “protein” are used interchangeably herein to refer to a polymer of amino acid residues. The terms apply to naturally occurring amino acid polymers as well as amino acid polymers in which one or more amino acid residues is a non-naturally occurring amino acid, for example, an amino acid analog. As used herein, the terms encompass amino acid chains of any length, including full length proteins, wherein the amino acid residues are linked by covalent peptide bonds.

In one embodiment, the present technology relates to Smad7 protein compositions. In addition to the entire Smad7 molecule, the present technology also relates to truncated portions and fragments of the polypeptide that retain one or more activity associated with Smad7 (including but not limited to, PTD-Smad7(203-426), PTD-Smad7(259-426), and PTD-Smad7(203-217), such as, but not limited to, increasing proliferation, reducing or inhibiting cell death, reducing excessive inflammation, preventing DNA damage, and/or increasing cell migration, as well as treating or preventing one or more disease or disorders in which such treatment would be helpful as further discussed herein. Such diseases and/or disorders may include but are not limited to acute (e.g., through surgery, combat, trauma, diabetes) and chronic wounds (e.g., ulcers, such as diabetic, pressure, venous), scarring (including keloid and hypertrophic scarring), fibrosis including lung fibrosis and radiation-induced lung fibrosis in lung cancer patients, and aberrant healing, mucositis (e.g., oral and/or gastro-intestinal, and cancer treatment-induced oral mucositis), stomatitis including recurrent aphthous stomatitis (Canker sore), proctitis, autoimmune disease (e.g., psoriasis, arthritis), radiation-induced dermatitis (radiodermatitis or RT dermatitis), atopic dermatitis, contact dermatitis, allergic dermatitis, Interstitial lung fibrosis (ILF) Radiation Pneumonitis leading to Pulmonary Fibrosis (e.g. due to cancer treating radiation). Such activities can be assessed using one or more assays including, but not limited to, the ability to block phosphorylation of Smad2 and/or nuclear translocation of the NF-κB p50 subunit, increase cell proliferation, reduce apoptosis and/or radiation-induced DNA damage, reduce inflammation and/or angiogenesis, promote healing in oral mucositis, surgical wounds, diabetes wounds, and/or wounds associated with chronic inflammation in mice.

Protein fragments may be generated by genetic engineering of translation stop sites within the coding region (discussed below). Alternatively, treatment of the Smad7 molecule with proteolytic enzymes, known as proteases, can produces a variety of N-terminal, C-terminal and internal fragments. These fragments may be purified according to known methods, such as precipitation (e.g., ammonium sulfate), HPLC, ion exchange chromatography, affinity chromatography (including immunoaffinity chromatography) or various size separations (sedimentation, gel electrophoresis, gel filtration).

As used herein, reference to an isolated protein or polypeptide in the present embodiments include full-length proteins, fusion proteins, chimeric proteins, or any fragment (truncated form, portion) or homologue of such a protein. More specifically, an isolated protein can be a protein (including a polypeptide or peptide) that has been removed from its natural milieu (i.e., that has been subject to human manipulation), and can include, but is not limited to, purified proteins, partially purified proteins, recombinantly produced proteins, proteins complexed with lipids, soluble proteins, synthetically produced proteins, and isolated proteins associated with other proteins. As such, “isolated” does not reflect the extent to which the protein has been purified. Preferably, an isolated protein is produced recombinantly.

Variants of Smad7 are also provided—these can be substitutional, insertional or deletion variants. Deletion variants lack one or more residues of the native protein that are not essential for activity, including the truncation mutants described above and herein. Substitutional variants typically contain the exchange of one amino acid for another at one or more sites within the protein, and may be designed to modulate one or more properties of the polypeptide, such as stability against proteolytic cleavage and/or translation and/or transcription (protein expression), without the loss of other functions or properties. Substitutions of this kind preferably are conservative, that is, one amino acid is replaced with one of similar shape and charge. Conservative substitutions are well known in the art and include, for example, each amino acid can be changed or substituted with a different amino acid. In making substitutional variants, the hydropathic index, hydrophilicity, charge and size are normally considered.

Specifically-contemplated deletion variants of Smad7 include truncations and fragments, for example, including polypeptide molecules having N-terminal sequences, but not C-terminal sequences, having C-terminal sequences but not N-terminal sequences, or having internal sequences, but not N-terminal or C-terminal sequences. Specifically, contemplated Smad7 polypeptide truncations or fragments include, but are not limited to, molecules including amino acid residues 203-426, 259-426, and 203-217 corresponding to the native human Smad7 protein sequence (SEQ ID NO:1).

The term “truncated” as used herein in reference to protein sequences refers to a molecule that contains the natural N-terminus of a corresponding protein (with or without a cleaved leader sequence), but lacks one or more amino acids starting from the C-terminus of the molecule, or a molecule that contains the natural C-terminus of a corresponding protein (with or without a cleaved leader sequence), but lacks one or more amino acids starting from the N-terminus of the molecule (including but not limited to, PTD-Smad7(203-426) and PTD-Smad7(259-426)). In some embodiments, molecules lacking at least about 25, at least about 50, at least about 75, at least about 100, at least about 125, at least about 150, at least about 200, at least about 250, at least about 300, or at least about 350, or at least about 400 amino acids from one or the other terminus are specifically provided. In some embodiments, a “truncated” molecule is biologically active, having one or more of the Smad7 activities described herein.

The term “fragment” as used herein in reference to polypeptide sequences refers to a molecule containing contiguous residues of a full-length sequence but lacking some N-terminal and/or C-terminal residues of the full-length sequence. In some embodiments, a “fragment” includes a portion of one or more of the full-length sequences described herein. In some embodiments, the “fragment” does not include sequences encoding either the N-terminal or the C-terminal, but only internal fragments. In some embodiments, a “fragment” encodes a polypeptide that is biologically active, having one or more of the Smad7 activities described herein (including but not limited to, PTD-Smad7(203-217). In some embodiments, polypeptide fragments have at least about 15, 20, 25, 30, 35, 40, 45, 50, 55, 60, 65, 70, 75, 80, 85, 90, 95, 100, 150, 200, 250, 300, 350, 400, 450, 500, 550, 600, 650, 700, 750, 800, 850, 900, 950, 1000, 1050, 1100, 1150 amino acids.

A specialized kind of variant is the fusion protein. This molecule generally has all or a substantial portion of the native molecule, linked at the N- or C-terminus, to all or a portion of a second polypeptide. However, in some embodiments, the fusion protein may include any one of the fragments and/or truncated (N-terminal, C-terminal) Smad7 proteins described throughout the disclosure (including but not limited to, PTD-Smad7(203-426), PTD-Smad7(259-426), and PTD-Smad7(203-217). For example, fusions may employ leader sequences from other species to permit the recombinant expression of a protein in a heterologous host. Another useful fusion includes the addition of an optional functionally active domain, such as but not limited to an antibody epitope and/or a purification tag (e.g., V5: GKPIPNPLLGLDST (SEQ ID NO: 3); Flag: KYKDDDDK (SEQ ID NO: 4); HA: YPYDVPDYA (SEQ ID NO: 5)). Another type of fusion includes attaching a domain that can act as the target for an activating or inactivating ligand, thereby permitting control of the fusion protein's function once delivered to a subject. Such domains include, for example, steroid ligand binding (e.g., ER, PR, GR), which can be activated by small molecules, e.g., 4-hydroxyl tamoxifen or RU486 that are either uniquely able to activate those steroid ligand binding domains and/or do not exist in nature and will therefore enable full control of the Smad7 function by the presence of these small molecules.

Another form of a fusion protein is a fusion of a Smad7 protein, including Smad7 fragments, with a protein transduction domain (PTD), also called a cell delivery domain or cell transduction domain. Such domains have been described in the art and are generally characterized as short amphipathic or cationic peptides and peptide derivatives, often containing multiple lysine and arginine resides (Fischer, Med. Res. Rev. 27:755-795 (2007)). The PTD may be one or more variants of Tat protein from HIV (GRKKRRQRRR (SEQ ID NO: 6), YGRKKRRQRRR (SEQ ID NO: 7), or GRKKRRQ (SEQ ID NO: 8)) or alternatively, HSV VP16. Alternate forms of Tat may be used. The PTD may be PEP (KETWWETWWTEWSQPKKKRKV (SEQ ID NO:28)) or functional variants thereof. A linker may be used to connect one or more PTDs and the Smad7 protein. The PTD (optionally Tat) is fused or linked in frame to the N-terminal and/or C-terminal end of any one of the Smad7 full-length, fragments, and/or truncated (N-terminal, C-terminal) proteins described above (including but not limited to, PTD-Smad7(203-426), PTD-Smad7(259-426), and PTD-Smad7(203-217). Other examples of PTDs useful as fusion proteins with Smad7 proteins in the methods of this disclosure are shown in the following table:

PROTEIN TRANSDUCTION DOMAINS

| | SEQ ID |
|---|---|
| | NO: |
| GALFLGWLGAAGSTMGAKK | 9 |
| KRKV | |
| RQIKIWFQNRRMKWKK | 10 |
| RRMKWKK | 11 |
| RRWRRWWRRWWRRWRR | 12 |
| RGGRLSYSRRRFSTSTGR | 13 |
| YGRKKRRQRRR | 7 |
| RKKRRQRRR | 14 |
| YARAAARQARA | 15 |
| RRRRRRRR | 16 |
| KKKKKKKK | 17 |
| GWTLNSAGYLLGKINLKALA | 18 |
| ALAKXIL | |
| LLILLRRRIRKQANAHSK | 19 |
| SRRHHCRSKAKRSRHH | 20 |
| NRARRNRRRVR | 21 |
| RQLRIAGRRLRGRSR | 22 |
| KLIKGRTPIKFGK | 23 |
| RRIPNRRPRR | 24 |
| KLALKLALKALKAALKLA | 25 |
| KLAKLAKKLAKLAK | 26 |
| GALFLGFLGAAGSTNGAWSQ | 27 |
| PKKKRKV | |
| KETWWETWWTEWSQPKKKR | 28 |
| KV | |
| LKKLLKKLLKKLLKKLLKKL | 29 |
| QAATATRGRSAASRPTERPR | 30 |
| APARSASRPRRPVE | |
| MGLGLHLLVLAAALQGAKS | 31 |
| KRKV | |
| AAVALLPAVLLALLAPAAA | 32 |
| NYKKPKL | |
| MANLGYWLLALFVTMWTD | 33 |
| VGLCKKRPKP | |
| LGTYTQDFNKFHTFPQTAIG | 34 |
| VGAP | |
| DPKGDPKGVTVTVTVTVTG | 35 |
| KGDPXPD | |
| PPPPPPPPPPPPPP | 36 |
| VRLPPPVRLPPPVRLPPP | 37 |
| PRPLPPPRPG | 38 |
| SVRRRPRPPYLPRPRPPPFFPP | 39 |
| RLPPRIPP | |
| TRSSRAGLQFPVGRVHRLLR | 40 |
| K | |
| GIGKFLHSAKKFGKAFVGEI | 41 |
| MNS | |
| KWKLFKKIEKVGQNIRDGII | 42 |
| KAGPAVAVVGQATQIAK | |
| ALWMTLLKKVLKAAAKAA | 43 |
| LNAVLVGANA | |
| GIGAVLKVLTTGLPALISWIK | 44 |
| RKRQQ | |
| INLKALAALAKKIL | 45 |
| GFFALIPKIISSPLPKTLLSAV | 46 |
| GSALGGSGGQE | |
| LAKWALKQGFAKLKS | 47 |
| SMAQDIISTIGDLVKWIIQTV | 48 |
| NXFTKK | |
| LLGDFFRKSKEKIGKEFKRIV | 49 |
| QRIKQRIKDFLANLVPRTES | |
| PAWRKAFRWAWRMLKKAA | 50 |
| KLKLKLKLKLKLKLKLKL | 51 |

In particular embodiments, the present technology provides for sequence variants of Smad7 in which one or more residues have been altered. For example, in one embodiment, the methionine residue found at position 216 of the human Smad7 sequence is modified to a leucine residue (ATG to CTG).

### C. Methods of Treatment

PTD-Smad7 and biologically active fragments and derivatives thereof (including but not limited to, PTD-Smad7(203-426), PTD-Smad7(259-426), and PTD-Smad7(203-217) treatable diseases and disorders may include those including one or more of reduced cell proliferation, reduced cell migration, increased cell death, excessive inflammation, and/or DNA damage. Smad7-related diseases and disorders may include those where treatment with a PTD-Smad7 protein and biologically active fragments and derivatives thereof (including but not limited to, PTD-Smad7(203-426), PTD-Smad7(259-426), and PTD-Smad7(203-217) that have one or more activities including but not limited to increasing proliferation, reducing or inhibiting cell death, reducing excessive inflammation, preventing DNA damage, and/or increasing cell migration is helpful. Such diseases and/or disorders may include but are not limited to acute (e.g., through surgery, combat, trauma, diabetic) and chronic wounds (e.g., ulcers, such as diabetic, pressure, venous), scarring (including keloid and hypertrophic scarring), fibrosis including lung fibrosis and radiation-induced lung fibrosis in lung cancer patients, and aberrant healing, mucositis (e.g., oral and/or gastro-intestinal, and cancer treatment-induced oral mucositis), stomatitis including recurrent aphthous stomatitis (Canker sore), proctitis, autoimmune disease (e.g., psoriasis, arthritis), radiation-induced dermatitis (radiodermatitis or RT dermatitis), atopic dermatitis, contact dermatitis, allergic dermatitis, Interstitial lung fibrosis (ILF) Radiation Pneumonitis leading to Pulmonary Fibrosis (e.g. due to cancer treating radiation), and cancer.

In some embodiments, one or more of the diseases and or disorders described herein may be prevented, treated, and/or ameliorated by providing to a subject in need of such treatment a therapeutically effective amount of one or more of the PTD-Smad7 proteins (e.g., full-length or biologically active truncated (e.g., N-terminal or C-terminal) or fragment thereof including but not limited to, PTD-Smad7(203-426), PTD-Smad7(259-426), and PTD-Smad7(203-217)) described in the disclosure. In some embodiments, the one or more Smad7 proteins are fusion proteins including a PTD domain. In some embodiments, the one or more Smad7 proteins includes Leu216. In some embodiments, the Smad7 proteins make part of a pharmaceutical composition including one or more pharmaceutically acceptable excipients.

In some embodiments, one or more of the diseases and or disorders described herein may be prevented, treated, and/or ameliorated by providing to a subject in need of such treatment a therapeutically effective amount of one or more of the nucleic acid molecules encoding one or more PTD-Smad7 proteins (e.g., full-length or biologically active truncated (e.g., N-terminal or C-terminal) or fragment thereof including but not limited to, PTD-Smad7(203-426), PTD-Smad7(259-426), and PTD-Smad7(203-217)) described in the disclosure. In some embodiments, the one or more nucleic acid molecules include codon-optimized nucleotide sequences and/or sequences that encode Leu216. In some embodiments, the one or more PTD-Smad7 nucleic acid molecules and biologically active fragments and derivatives thereof (including but not limited to, PTD-Smad7(203-426), PTD-Smad7(259-426), and PTD-Smad7(203-217) are provided to the subject in a construct including an expression vector. In some embodiments, the Smad7 nucleic acid molecules and biologically active fragments and derivatives thereof (optionally part of an expression vector) make part of a pharmaceutical composition including one or more pharmaceutically acceptable excipients.

The term “subject” or “patient” as used herein refers to persons or non-human animals in need of treatment and or prevention using one or more of the treatments described herein. In some embodiments, non-human animals include laboratory animals such as monkeys, mice, rats, and rabbits, domestic pets such as dogs and cats, and livestock such as cattle, horses, pigs, goats and sheep.

1. Chronic Wounds

A chronic wound is a wound that does not heal in an orderly set of stages and in a predictable amount of time the way most wounds do; wounds that do not heal within three months are often considered chronic. Chronic wounds seem to be detained in one or more of the phases of wound healing. For example, chronic wounds often remain in the inflammatory stage for too long. In acute wounds, there is a precise balance between production and degradation of molecules such as collagen; in chronic wounds this balance is lost and degradation plays too large a role.

As described in more detail elsewhere herein, PTD-Smad7 and one or more biologically active fragments and derivatives thereof (including but not limited to, PTD-Smad7(203-426), PTD-Smad7(259-426), and PTD-Smad7(203-217) have been shown to enhance wound healing in a mouse skin model, a diabetic mouse wound model, a radiation dermatitis mouse model, and a mucosal model. Application of PTD-Smad7 and biologically active fragments and derivatives thereof (including but not limited to, PTD-Smad7(203-426), PTD-Smad7(259-426), and PTD-Smad7(203-217) was effective through a topical route, which is desirable for wound treatment, as well as with local injections. Although not intending to be bound by theory, it is believed that PTD-Smad7 and biologically active fragments and derivatives thereof (including but not limited to, PTD-Smad7(203-426), PTD-Smad7(259-426), and PTD-Smad7(203-217) may act to treat or ameliorate wounds, including chronic wounds, through multiple routes, which may include reducing inflammation, increasing cell proliferation (e.g., keratinocytes), increasing cell migration (e.g., keratinocytes), or reducing fibrosis (e.g., through modulation of collagen), among others.

Chronic wounds may never heal or may take years to do so. These wounds cause patients severe emotional and physical stress as well as creating a significant financial burden on patients and the whole healthcare system. Acute and chronic wounds are at opposite ends of a spectrum of wound healing types that progress toward being healed at different rates. The vast majority of chronic wounds can be classified into three categories: venous ulcers, diabetic wounds and ulcers, and pressure ulcers. A small number of wounds that do not fall into these categories may be due to causes such as radiation poisoning (e.g., radiation-induced dermatitis) or ischemia.

Venous and Arterial Ulcers.

Venous ulcers, which usually occur in the legs, account for about 70% to 90% of chronic wounds and mostly affect the elderly. They are thought to be due to venous hypertension caused by improper function of valves that exist in the veins to prevent blood from flowing backward. Ischemia results from the dysfunction and, combined with reperfusion injury, causes the tissue damage that leads to the wounds.

Diabetic Wounds, Including Ulcers.

Another major cause of chronic wounds, diabetes, is increasing in prevalence. Diabetics have a 15% higher risk for amputation than the general population due to chronic wounds, including ulcers. PTD-Smad7 and biologically active fragments and derivatives thereof (including but not limited to, PTD-Smad7(203-426), PTD-Smad7(259-426), and PTD-Smad7(203-217) have been shown herein to be more effective at treating diabetic wounds than the currently FDA-approved treatment, Regranax™. Diabetes causes neuropathy, which inhibits nociception and the perception of pain. Thus, patients may not initially notice small wounds to legs and feet, and may therefore fail to prevent infection or repeated injury. Further, diabetes causes immune compromise and damage to small blood vessels, preventing adequate oxygenation of tissue, which can cause chronic wounds. Pressure also plays a role in the formation of diabetic ulcers.

Pressure Ulcers.

Another leading type of chronic wounds is pressure ulcers, which usually occur in people with conditions such as paralysis that inhibit movement of body parts that are commonly subjected to pressure such as the heels, shoulder blades, and sacrum. Pressure ulcers are caused by ischemia that occurs when pressure on the tissue is greater than the pressure in capillaries, and thus restricts blood flow into the area. Muscle tissue, which needs more oxygen and nutrients than skin does, shows the worst effects from prolonged pressure. As in other chronic ulcers, reperfusion injury damages tissue.

Chronic wounds may affect only the epidermis and dermis, or they may affect tissues all the way to the fascia. They may be formed originally by the same things that cause acute wounds, such as surgery or accidental trauma, or they may form as the result of systemic infection, vascular, immune, or nerve insufficiency, or comorbidities such as neoplasias or metabolic disorders. Although not intending to be bound by theory, the reason a wound becomes chronic is that the body's ability to deal with the damage is overwhelmed by factors such as repeated trauma, continued pressure, ischemia, or illness. Some of the major factors that lead to chronic wounds include, but are not limited to, ischemia, reperfusion injury, and bacterial colonization.

Ischemia.

Ischemia is an important factor in the formation and persistence of wounds, especially when it occurs repetitively (as it usually does) or when combined with a patient's old age. Ischemia causes tissue to become inflamed and cells to release factors that attract neutrophils such as interleukins, chemokines, leukotrienes, and complement factors.

While they fight pathogens, neutrophils also release inflammatory cytokines and enzymes that damage cells. One of their important functions is to produce Reactive Oxygen Species (ROS) to kill bacteria, for which they use an enzyme called myeloperoxidase. The enzymes and ROS produced by neutrophils and other leukocytes damage cells and prevent cell proliferation and wound closure by damaging DNA, lipids, proteins, the ECM, and cytokines that speed healing. Neutrophils remain in chronic wounds for longer than they do in acute wounds, and contribute to the fact that chronic wounds have higher levels of inflammatory cytokines and ROS. Because wound fluid from chronic wounds has an excess of proteases and ROS, the fluid itself can inhibit healing by inhibiting cell growth and breaking down growth factors and proteins in the ECM.

Bacterial Colonization.

Since more oxygen in the wound environment allows white blood cells to produce ROS to kill bacteria, patients with inadequate tissue oxygenation, for example, those who suffered hypothermia during surgery, are at higher risk for infection. The host's immune response to the presence of bacteria prolongs inflammation, delays healing, and damages tissue. Infection can lead not only to chronic wounds but also to gangrene, loss of the infected limb, and death of the patient.

Like ischemia, bacterial colonization and infection damage tissue by causing a greater number of neutrophils to enter the wound site. In patients with chronic wounds, bacteria with resistance to antibiotics may have time to develop. In addition, patients carrying drug resistant bacterial strains, such as methicillin-resistant Staphylococcus aureus (MRSA), have more chronic wounds.

Growth Factors and Proteolytic Enzymes.

Chronic wounds also differ in makeup from acute wounds in that their levels of proteolytic enzymes such as elastase and matrix metalloproteinases (MMPs) are higher, while their concentrations of growth factors such as Platelet-derived growth factor and Keratinocyte Growth Factor are lower.

Because growth factors (GFs) are imperative in wound healing, inadequate GF levels may be an important factor in chronic wound formation. In chronic wounds, the formation and release of growth factors may be prevented, the factors may be sequestered and unable to perform their metabolic roles, or degraded in excess by cellular or bacterial proteases.

Chronic wounds such as diabetic and venous ulcers are also caused by a failure of fibroblasts to produce adequate ECM proteins and by keratinocytes to epithelialize the wound. Fibroblast gene expression is different in chronic wounds than in acute wounds.

Although all wounds require a certain level of elastase and proteases for proper healing, too high a concentration is damaging. Leukocytes in the wound area release elastase, which increases inflammation, destroys tissue, proteoglycans, and collagen, and damages growth factors, fibronectin, and factors that inhibit proteases. The activity of elastase is increased by human serum albumin, which is the most abundant protein found in chronic wounds. However, chronic wounds with inadequate albumin are especially unlikely to heal, so regulating the wound's levels of that protein may in the future prove helpful in healing chronic wounds.

Excess matrix metalloproteinases, which are released by leukocytes, may also cause wounds to become chronic. MMPs break down ECM molecules, growth factors, and protease inhibitors, and thus increase degradation while reducing construction, throwing the delicate compromise between production and degradation out of balance.

2. Acute Wounds/Trauma

Physical trauma is a serious and body-altering physical injury, such as the removal of a limb. Blunt force trauma, a type of physical trauma caused by impact or other force applied from or with a blunt object, whereas penetrating trauma is a type of physical trauma in which the skin or tissues are pierced by an object. Trauma can also be described as both unplanned, such as an accident, or planned, in the case of surgery. Both can be characterized by mild to severe tissue damage, blood loss and/or shock, and both may lead to subsequent infection, including sepsis. The present technology provides treatment of trauma, including both pre-treatment (in the case of a medical procedure) and treatment after trauma injury has occurred.

As described in more detail elsewhere herein (and briefly mentioned above), PTD-Smad7 and biologically active fragments and derivatives thereof (including but not limited to, PTD-Smad7(203-426), PTD-Smad7(259-426), and PTD-Smad7(203-217) have been shown to enhance wound healing in one or more of a mouse skin model, a mouse model of diabetic wounds, a radiation dermatitis model, and a mucosal model. Application of PTD-Smad7 and biologically active fragments and derivatives thereof (including but not limited to, PTD-Smad7(203-426), PTD-Smad7(259-426), and PTD-Smad7(203-217) was effective in one or more of these models through a topical route, which is desirable for wound treatment, as well as through local injection. Although not intending to be bound by theory, it is believed that PTD-Smad7 and biologically active fragments and derivatives thereof (including but not limited to, PTD-Smad7(203-426), PTD-Smad7(259-426), and PTD-Smad7(203-217) may act to treat or ameliorate wounds through multiple routes, which may include one or more of reducing inflammation, increasing cell proliferation (e.g., keratinocytes), increasing cell migration (e.g., keratinocytes), or reducing fibrosis (e.g., through modulation of collagen), among others. As described briefly below, reduced inflammation could significantly contribute to accelerated wound healing, optionally through reduced angiogenesis and collagen production and/or reduced leukocyte infiltration leading to reduction of cytokines and chemokines normally released by leukocytes, which are angiogenic and fibrogenic. Temporal treatment with Smad7 may allow early stage angiogenesis and collagen production required for wound repair, while preventing prolonged angiogenesis and collagen production. These changes could potentially accelerate wound stromal remodeling and prevent excessive scarring due to unresolved inflammation or collagen overproduction. For surgical procedures (as well as everyday injuries), particularly where the potential for scarring is an issue (including, for example, Keloid and hypertrophic scar formation), treatment with the Smad7 protein fusion constructs, and biologically active fragments and derivatives thereof (including but not limited to, PTD-Smad7(203-426), PTD-Smad7(259-426), and PTD-Smad7(203-217) of this disclosure may be beneficial.

Oral Ulcers.

A mouth ulcer (also termed an oral ulcer, or a mucosal ulcer) is an ulcer that occurs on the mucous membrane of the oral cavity. More plainly, a mouth ulcer is a sore or open lesion in the mouth. Mouth ulcers are very common, occurring in association with many diseases and by many different mechanisms, but usually there is no serious underlying cause. The two most common causes of oral ulceration are local trauma (e.g., rubbing from a sharp edge on a filling) and aphthous stomatitis (“canker sores”), a condition characterized by recurrent formation of oral ulcers for largely unknown reasons. Some consider ulcers on the lips or on the skin around the mouth to be included under the general term oral ulceration (e.g., an ulcer left by rupture of a blister caused by herpes labialis, i.e., a cold sore). Mouth ulcers often cause pain and discomfort, and may alter the person's choice of food while healing occurs (e.g., avoiding acidic or spicy foods and beverages). They may occur singly or multiple ulcers may occur at the same time (a “crop” of ulcers). Once formed, the ulcer may be maintained by inflammation and/or secondary infection. Rarely, a mouth ulcer that does not heal for many weeks may be a sign of oral cancer. Other causes include burns, chemical injury, or infection.

A mucosal ulcer is an ulcer which specifically occurs on a mucous membrane. An ulcer is a tissue defect which has penetrated the epithelial-connective tissue border, with its base at a deep level in the submucosa, or even within muscle or periosteum. An ulcer is a deeper breech of the epithelium than an erosion or an excoriation, and involves damage to both epithelium and lamina propria. An erosion is a superficial breach of the epithelium, with little damage to the underlying lamina propria. A mucosal erosion is an erosion which specifically occurs on a mucous membrane. Only the superficial epithelial cells of the epidermis or of the mucosa are lost, and the lesion can reach the depth of the basement membrane. Erosions heal without scar formation. Excoriation is a term sometimes used to describe a breach of the epithelium which is deeper than an erosion but shallower than an ulcer. This type of lesion is tangential to the rete pegs and shows punctiform (small pinhead spots) bleeding, caused by exposed capillary loops.

Surgery.

Surgery uses operative manual and instrumental techniques on a patient to investigate and/or treat a pathological condition such as disease or injury, to help improve bodily function or appearance, or sometimes for some other reason. The present technology can address trauma resulting from surgeries, as defined further below.

As a general rule, a procedure is considered surgical when it involves cutting of a patient's tissues or closure of a previously sustained wound. Other procedures that do not necessarily fall under this rubric, such as angioplasty or endoscopy, may be considered surgery if they involve common surgical procedure or settings, such as use of a sterile environment, anesthesia, antiseptic conditions, typical surgical instruments, and suturing or stapling. All forms of surgery are considered invasive procedures; so-called noninvasive surgery usually refers to an excision that does not penetrate the structure being addressed (e.g., laser ablation of the cornea) or to a radiosurgical procedure (e.g., irradiation of a tumor). Surgery can last from minutes to hours.

Surgical procedures are commonly categorized by urgency, type of procedure, body system involved, degree of invasiveness, and special instrumentation. Elective surgery is done to correct a non-life-threatening condition, and is carried out at the patient's request, subject to the surgeon's and the surgical facility's availability. Emergency surgery is surgery which must be done quickly to save life, limb, or functional capacity. Exploratory surgery is performed to aid or confirm a diagnosis. Therapeutic surgery treats a previously diagnosed condition.

Amputation involves cutting off a body part, usually a limb or digit. Replantation involves reattaching a severed body part. Reconstructive surgery involves reconstruction of an injured, mutilated, or deformed part of the body. Cosmetic surgery is done to improve the appearance of an otherwise normal structure. Excision is the cutting out of an organ, tissue, or other body part from the patient. Transplant surgery is the replacement of an organ or body part by insertion of another from different human (or animal) into the patient. Removing an organ or body part from a live human or animal for use in transplant is also a type of surgery.

When surgery is performed on one organ system or structure, it may be classified by the organ, organ system or tissue involved. Examples include cardiac surgery (performed on the heart), gastrointestinal surgery (performed within the digestive tract and its accessory organs), and orthopedic surgery (performed on bones and/or muscles).

Minimally invasive surgery involves smaller outer incision(s) to insert miniaturized instruments within a body cavity or structure, as in laparoscopic surgery or angioplasty. By contrast, an open surgical procedure requires a large incision to access the area of interest. Laser surgery involves use of a laser for cutting tissue instead of a scalpel or similar surgical instruments. Microsurgery involves the use of an operating microscope for the surgeon to see small structures. Robotic surgery makes use of a surgical robot, such as Da Vinci or Zeus surgical systems, to control the instrumentation under the direction of the surgeon.

3. Autoimmune/Inflammatory Disease

The present technology contemplates the treatment of a variety of autoimmune and/or inflammatory disease states such as spondyloarthropathy, ankylosing spondylitis, psoriatic arthritis, reactive arthritis, enteropathic arthritis, ulcerative colitis, Crohn's disease, irritable bowel disease, inflammatory bowel disease, rheumatoid arthritis, juvenile rheumatoid arthritis, familial Mediterranean fever, amyotrophic lateral sclerosis, Sjogren's syndrome, early arthritis, viral arthritis, multiple sclerosis, or psoriasis. The diagnosis and treatment of these diseases are well documented in the literature.

In general, autoimmune diseases are associated with an overactive immune response of a body against substances and tissues normally present in the body, and not normally the focus of an immune response. There are more than 80 types of autoimmune diseases, some of which have similar symptoms, and they may arise from a similar underlying cause. The classic sign of an autoimmune disease is inflammation, which as disclosed herein is amenable to treatment with PTD-Smad7 (optionally biologically active fragments and derivatives thereof (including but not limited to, PTD-Smad7(203-426), PTD-Smad7(259-426), and PTD-Smad7(203-217) compositions. PTD-Smad7(203-426) or Tat-Smad7(203-426) has been shown herein to be effective in a model of psoriasis.

4. Chemotherapy, Radiotherapy and Cytokine Therapy Toxicity

Various forms of cancer therapy, including chemotherapy, radiation, and cytokines, are associated with toxicity, sometimes severe, in the cancer patient. The methods of this disclosure are also effective in treating or reducing the severity of this toxicity by administration of the pharmaceutical compositions of the present technology, thereby reducing or alleviating discomfort on the part of the patient, as well as permitting higher doses of the therapy. Thus, the methods of this disclosure include methods of treating, preventing, and ameliorating radiation-induced injuries, including, for example, radiation-induced dermatitis (“radiodermatitis” or “RT dermatitis”), and Radiation Pneumonitis leading to Pulmonary Fibrosis (e.g. due to cancer treating radiation). PTD-Smad7(203-426) or Tat-Smad7(203-426) is shown herein to be effective at treating radiodermatitis in a mouse model.

As described at length throughout this disclosure, it has been found that PTD-Smad7 and biologically active fragments and derivatives thereof (including but not limited to, PTD-Smad7(203-426), PTD-Smad7(259-426), and PTD-Smad7(203-217) acts to heal as well as to prevent oral mucositis in a mouse model. PTD-Smad7 and one or more biologically active fragments and derivatives thereof (including but not limited to, PTD-Smad7(203-426), PTD-Smad7(259-426), and PTD-Smad7(203-217) was shown to be more effective than palifermin, the existing approved drug for preventing oral mucositis, in direct comparisons.

Oral cancer, the 6^{th} most common cancer worldwide, is a subtype of head and neck cancer, and includes any cancerous tissue growth located in the oral cavity. It may arise as a primary lesion originating in any of the oral tissues, by metastasis from a distant site of origin, or by extension from a neighboring anatomic structure, such as the nasal cavity or the oral cancers may originate in any of the tissues of the mouth, and may be of varied histologic types: teratoma, adenocarcinoma derived from a major or minor salivary gland, lymphoma from tonsillar or other lymphoid tissue, or melanoma from the pigment-producing cells of the oral mucosa. There are several types of oral cancers, but around 90% are squamous cell carcinomas, originating in the tissues that line the mouth and lips. Oral or mouth cancer most commonly involves the tongue. It may also occur on the floor of the mouth, cheek lining, gingiva (gums), lips, or palate (roof of the mouth). Most oral cancers look very similar under the microscope and are called squamous cell carcinoma. These are malignant and tend to spread rapidly.

Over 80% of oral cancer patients are treated with radiation therapy and at least 75% of these individuals will develop oral mucositis (i.e., cancer treatment-induced oral mucositis). Oral mucositis is a chronic oral ulceration. This disease frequently occurs in radiation-treated patients of all cancer types, including but not limited to patients who are radiation-treated for organ transplants (to eliminate rejection of the transplants), and patients undergoing routine chemotherapy. Severe oral mucositis is extremely painful and impairs food/liquid intake, hence is often the most severe complication of cancer therapy. Oral mucositis is a major factor in determining the maximum dose possible of radiation and chemotherapy to the head and neck region; it can significantly complicate cancer treatment, extend hospitalization, decrease quality of life and increase costs.

Currently, there is no established therapy to effectively treat severe oral mucositis. To date, palifermin (KEPIVANCE®), a recombinant protein of human keratinocyte growth factor (KGF), is the only FDA approved drug for intravenous (i.v.) injections for severe oral mucositis in bone-marrow transplant patients, and its use in cancer patients remains to be determined. It is also used for prevention of oral mucositis. Hence, this drug is available for only 4% of the at-risk population. It also suffers from the need for medical service providers due to the i.v. administration route. Other potential therapies include topical rinses, such as viscous 2% lidocaine rinses, or baking soda and saline solutions, or a cocktail solution, for instance BAX (lidocaine, diphenhyramine, sorbitol and MYLANTA®). Other investigative or mucoprotective adjuvant therapies include, but are not limited to, beta carotene, tocopherol, laser irradiation, prophylactic brushing the oral mucosa with silver-nitrate, misoprostol, leucovorin, systemic KGF, pentoxifylline, allopurinol mouthwash, systemic sucralfate, chlorhexidine gluconate, and cryotherapy.

Chemotherapy- and radiation-induced gut mucositis is an inflammatory condition that arises as a result of the acute death of rapidly dividing intestinal epithelial cells. Most chemotherapeutic drugs used for treatment of solid tumors, alone, in a combination of drugs, or with radiation, will result in the death of a large number of intestinal epithelial cells. The clinical manifestations of the ensuing mucositis include digestive symptoms such as nausea and vomiting, serious diarrhea, acute weight loss and wasting. This is fast becoming one of the limiting factors for administering chemotherapy for many cancer patients. The ability of Tat-Smad7 to protect intestinal epithelial cells from either chemotherapeutic agents, radiation, or a combinations of those, will significantly decrease the undesirable side effects of cancer therapies, and enable more aggressive ways to treat the disease with existing tools.

Bone marrow failure syndromes are a set of conditions that develop when the hematopoietic stem cell compartment is compromised and fails to give rise to normal cell types. Bone marrow failure occurs as a result of inherited genetic abnormalities, exposure to a noxious substance, such as toxins, chemicals or viruses. Although the nature and identity of environmental factors that can lead to the development of acquired bone marrow failure is still not completely understood, a few factors have been linked to the development of acquired bone marrow failure among military personnel including exposure to mustard gas, ionizing radiation, and infectious agents such as visceral leishmaniasis or African trypanosomiasis. The best approach for management of bone marrow failure syndromes is still the transplantation of hematopoietic stem cells (HSCs), unless a sufficient number of the remaining resident bone marrow HSCs can be spared from these stresses and encouraged to repopulate the hematopoietic compartment. The modulation of Smad 7, as described here, should enable for the deliberate protection of the remaining resident HSCs in patients that exhibit clinical signs consistent with bone marrow failure.

5. Cancer

TGF-β and NF-κB activations are known to promote cancer invasion and metastasis. Currently, TGF-β inhibitors are in clinical trials for treating metastatic cancer and NF-κB inhibitors are used in cancer prevention. The demonstrated effect of Smad7 on blocking both TGF-β and NF-κB signaling present the possibility that it is an even stronger anti-cancer/anti-metastasis agent than other inhibitors that inhibit only one of these two pathways. Smad7 has been shown to prevent angiogenesis and fibrogenesis, and may therefore be particularly useful in situations where the tumor needs to develop a blood supply and/or stroma.

The cancer may be selected from the group consisting of brain, lung, liver, spleen, kidney, lymph node, small intestine, pancreas, blood cells, colon, stomach, breast, endometrium, prostate, testicle, cervix, uterus, ovary, skin, head & neck, esophagus, bone marrow and blood cancer. The cancers may be metastatic or primary, recurrent or multi-drug resistant. In some embodiments, the cancer is a solid tumor (organ tumor). Solid tumors refer to a mass of cells that grow in organ systems and can occur anywhere in the body. Two types of solid tumors include epithelial tumors (carcinomas) that occur in the epithelial tissue inside or outside an organ, and sarcomas (connective tissue tumors) that occur in connective tissue such as, but not limited to, muscles, tendons, fat, nerves and other connective tissues that support, surround, or connect structures and organs in the body. In some embodiments, the cancer is a liquid tumor or cancer of the blood, bone marrow, or lymph nodes. These tumors include, but are not limited to, leukemia, lymphoma, and myeloma. In the context of cancer therapy, the Smad7 constructs of this disclosure may be useful in treating and preventing secondary effects of cancer, often resulting from inflammatory conditions or wounds induced by the cancer or by the cancer treatment, such as lung fibrosis in lung cancer patients.

6. Scarring, Fibrosis, and Aberrant Healing

In addition to accelerated re-epithelialization (e.g., through increasing cell proliferation and/or increasing cell migration), PTD-Smad7(203-426) or Tat-Smad7(203-426) effects on wound stroma include one or more of reducing inflammation, angiogenesis, or collagen production, among others. Although not intending to be bound by theory these effects may be mediated through reduction of NF-κB signaling (evidenced by reduced p50), and blocking TGF-β signaling (evidenced by reduced pSmad2). As a result, reduced inflammation could significantly contribute to accelerated wound healing, optionally through reduced angiogenesis and collagen production and/or reduced leukocyte infiltration leading to reduction of cytokines and chemokines normally released by leukocytes, which are angiogenic and fibrogenic. Temporal treatment with PTD-Smad7 and biologically active fragments and derivatives thereof (including but not limited to, PTD-Smad7(203-426), PTD-Smad7(259-426), and PTD-Smad7(203-217) may allow early stage angiogenesis and collagen production required for wound repair, while preventing prolonged angiogenesis and collagen production. These changes could potentially accelerate wound stromal remodeling and prevent excessive scarring due to unresolved inflammation or collagen overproduction. Reduced inflammation following treatment with the PTD-Smad7 and biologically active fragments and derivatives thereof (including but not limited to, PTD-Smad7(203-426), PTD-Smad7(259-426), and PTD-Smad7(203-217) protein constructs of this disclosure may also effectively treat, prevent, and/or ameliorate lung fibrosis.

Thus, this disclosure also provides methods for treatment of a subject suffering from lung fibrosis comprising administering to the subject an effective amount of a PTD-Smad7 and biologically active fragments and derivatives thereof (including but not limited to, PTD-Smad7(203-426), PTD-Smad7(259-426), and PTD-Smad7(203-217) protein fusion construct disclosed herein, thereby treating the lung fibrosis. The subject may be suffering from interstitial lung fibrosis (ILF). The subject may be suffering from Radiation Pneumonitis leading to Pulmonary Fibrosis. The subject may be suffering from drug induced lung fibrosis. Similarly, the methods of this disclosure also provide for the use of a PTD-Smad7 and biologically active fragments and derivatives thereof (including but not limited to, PTD-Smad7(203-426), PTD-Smad7(259-426), and PTD-Smad7(203-217) protein fusion construct, or nucleic acid molecule encoding the same, for the manufacture of a medicament to treat lung fibrosis. In some embodiments, the lung fibrosis is ILF. In some embodiments, the lung fibrosis is drug- or radiation-induced lung fibrosis.

7. Stomatitis

Stomatitis is an inflammation of the mucous lining of any of the structures in the mouth, which may involve the cheeks, gums, tongue, lips, throat, and roof or floor of the mouth. Thus, stomatitis includes recurrent aphthous stomatitis (“Canker sore”). The inflammation can be caused by conditions in the mouth itself, such as poor oral hygiene, dietary protein deficiency, poorly fitted dentures, or from mouth burns from hot food or drinks, toxic plants, or by conditions that affect the entire body, such as medications, allergic reactions, radiation therapy, or infections. Severe iron deficiency anemia can lead to stomatitis. Iron is necessary for the upregulation of transcriptional elements for cell replication and repair. Lack of iron can cause the genetic downregulation of these elements, leading to ineffective repair and regeneration of epithelial cells, especially in the mouth and lips. This condition is also prevalent in people who have a deficiency in vitamin B₂ (Riboflavin), B₃ (Niacin), B₆ (Pyridoxine), B₉ (folic acid) or B₁₂ (cobalamine). When it also involves an inflammation of the gingiva (gums), it is called gingivostomatitis. It may also be seen in ariboflavinosis (riboflavin deficiency) or neutropenia.

Irritation and fissuring in the corners of the lips is termed angular stomatitis or angular cheilitis. In children, angular stomatitis is a frequent cause is repeated lip-licking and in adults it may be a sign of underlying iron deficiency anemia, or vitamin B deficiencies (e.g., B₂-riboflavin, B₉-folate or B₁₂-cobalamin), which in turn may be evidence of poor diets or malnutrition (e.g., celiac disease). Also, angular cheilitis can be caused by a patient's jaws at rest being “overclosed” due to edentulousness or tooth wear, causing the jaws to come to rest closer together than if the complete/unaffected dentition were present. This causes skin folds around the angle of the mouth which are kept moist by saliva which in turn favours infection; mostly by Candida albicans or similar species. Treatment usually involves the administration of topical nystatin or similar antifungal agents. Another treatment can be to correct the jaw relationship with dental treatment (e.g., dentures or occlusal adjustment).

Migratory stomatitis is a condition in which extensive areas in the oral cavity mucosa are affected by annular atrophic red lesions that are surrounded by a thin white rim. This is a relatively uncommon form of the geographic tongue condition, that, as opposed to migratory stomatitis, is confined to the dorsal and lateral aspects of the tongue mucosa only.

8. Proctitis

Proctitis is inflammation of the lining of the rectum, the lower end of the large intestine leading to the anus. With proctitis, inflammation of the rectal lining—called the rectal mucosa—is uncomfortable and sometimes painful. The condition may lead to bleeding or mucous discharge from the rectum, among other symptoms. Some causes of proctitis include, but are not limited to: sexually transmitted diseases (STDs), such as those that can be transmitted during anal sex (e.g., gonorrhea, chlamydia, syphilis, and herpes); non-STD infections from, for example, food borne bacteria (e.g., Salmonella and Shigella); anorectal trauma from, for example, anal sex or the insertion of objects or substances into the rectum (e.g., chemicals from enemas); ulcerative colitis and Crohn's disease or other inflammatory bowel diseases, may cause ulcers (e.g., sores) in the inner lining of the colon and rectum; radiation therapy, particularly of the pelvic area (e.g., rectal, ovarian, or prostate cancer) which may lead to rectal bleeding; antibiotics which lead to a loss of commensal bacteria allowing harmful bacteria (e.g., Clostridium difficile) to cause disease.

9. Allergic Dermatitis, Atopic Dermatitis, and Contact Dermatitis

Atopic Dermatitis (AD) is a genetically determined, reaginically (IgE) associated, chronic disease of the skin affecting approximately 8 million adults and children in the United States. In AD, the skin is dry, easily irritated, subject to immediate hypersensitivity type of allergic responses, typically scaly, often thickened, commonly red, frequently infected, sometimes exudative and above all itchy. Reaginic diseases, i.e. atopic diseases, are characterized by the capacity to form IgE antibodies, on a genetic basis, resulting in immediate hypersensitivity reactions upon exposure to many specific allergens, most prominent among which may be the house dust mite (Dermatophagoides pteronyssinus), but which also include pollens, molds and danders. Multiple genetic factors contribute to expression of this phenotype. Atopic susceptibility genes include those making several major histocompatibility complex (HLA) class II molecules, IL-4 receptor proteins and IgE high affinity receptor proteins.

Allergic dermatitis is the manifestation of an allergic response caused by contact with a substance. Although less common than contact dermatitis, allergic dermatitis is the most prevalent form of immunotoxicity found in humans. By its allergic nature, this form of dermatitis is a hypersensitive reaction that is atypical within the population. The symptoms of allergic dermatitis are very similar to the ones caused by contact dermatitis. The first sign of allergic contact dermatitis is the presence of the rash or skin lesion at the site of exposure. Depending on the type of allergen causing it, the rash can ooze, drain or crust and it can become raw, scaled or thickened. Also, it is possible that the skin lesion does not take the form of a rash but it may include papules, blisters, vesicles or even a simple red area. The main difference between the rash caused by allergic dermatitis and the one caused by contact dermatitis is that the latter tends to be confined to the area where the trigger touched the skin, whereas in allergic contact dermatitis the rash is more likely to be more widespread on the skin. Another characteristic of the allergic dermatitis rash is that it usually appears after a day or two after exposure to the allergen, unlike contact dermatitis that appears immediately after the contact with the trigger. Other symptoms may include itching, skin redness or inflammation, localized swelling and the area may become more tender or warmer. If left untreated, the skin may darken and become leathery and cracked. Pain can also be present.

The symptoms of allergic dermatitis may persist for as long as one month before resolving completely. Once an individual has developed a skin reaction to a certain substance it is most likely that they will have it for the rest of their life, and the symptoms will reappear when in contact with the allergen.

Contact dermatitis is responsible for over 5.6 million doctor visits each year in the United States and accounts for 15-20% of all occupational diseases. Eighty percent of contact dermatitis instances are due to irritants while in the other 20% the compound induces an immunologic cascade and are classified as allergic. Contact dermatitis and many hypersensitivity reactions of the skin are produced by haptens, in the form of low molecular weight molecules or metal ions, complexing with cellular proteins. Subsequently, these are processed into peptides and presented on the surface of antigen-presenting cells (APCs), typically Langerhans cells, the principle APC of the skin, residing in the epidermis. Once Langerhans undergo maturation, they migrate to the regional lymph node and present hapten-modified peptides in the context of major histocompatibility class I and II molecules to hapten-specific CD8+ and CD4+ T cells, respectively. Antigen-specific activation of T cells constitutes the sensitization phase of contact sensitivity responses. Upon subsequent exposure to hapten, the challenge phase, effector memory T cells migrate to the peripheral tissues harboring hapten-presenting APCs. Here antigen recognition induces the T cells to express various mediators of inflammation and cytotoxicity, ultimately causing dermatitis and tissue damage.

PTD-Smad7 and one or more biologically active fragments and derivatives thereof (including but not limited to, PTD-Smad7(203-426), PTD-Smad7(259-426), and PTD-Smad7(203-217) show activity in one or more models described herein.

10. Formulations and Routes of Administration

Where clinical applications are contemplated, it will be necessary to prepare pharmaceutical compositions—proteins, expression vectors, virus stocks, proteins and drugs—in a form appropriate for the intended application. Generally, this will entail preparing compositions that are essentially free of pyrogens, as well as other impurities that could be harmful to humans or animals.

PTD-Smad7 (and truncated variants) were purified extensively prior to use in animal models. PTD-Smad7 (and truncated versions) were prepared for topical and trans-mucosal application using a mixture of glycerol and PBS.

One will generally desire to employ appropriate salts and buffers to render delivery vectors stable and allow for uptake by target cells. Buffers also will be employed when recombinant cells are introduced into a patient. Aqueous compositions of the present technology comprise an effective amount of the vector to cells, dissolved or dispersed in a pharmaceutically acceptable carrier or aqueous medium. Such compositions also are referred to as inocula. The phrase “pharmaceutically or pharmacologically acceptable” refer to molecular entities and compositions that do not produce adverse, allergic, or other untoward reactions when administered to an animal or a human. As used herein, “pharmaceutically acceptable carrier” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the vectors or cells of the present technology, its use in therapeutic compositions is contemplated. Supplementary active ingredients also can be incorporated into the compositions.

The active compositions of the present technology may include classic pharmaceutical preparations. Administration of these compositions according to the present technology will be via any common route so long as the target tissue is available via that route. Such routes of administration may include oral parenteral (including intravenous, intramuscular, subcutaneous, intradermal, intra-articular, intra-synovial, intrathecal, intra-arterial, intracardiac, subcutaneous, intraorbital, intracapsular, intraspinal, intrastemal, and transdermal), nasal, buccal, urethral, rectal, vaginal, mucosal, dermal, or topical (including dermal, buccal, and sublingual). Alternatively, administration may be by orthotopic, intradermal, subcutaneous, intramuscular, intraperitoneal or intravenous injection. Such compositions would normally be administered as pharmaceutically acceptable compositions, described supra. Of particular interest is direct intratumoral administration, perfusion of a tumor, or administration local or regional to a tumor, for example, in the local or regional vasculature or lymphatic system, or in a resected tumor bed. Administration can also be via nasal spray, surgical implant, internal surgical paint, infusion pump, or via catheter, stent, balloon or other delivery device. The most useful and/or beneficial mode of administration can vary, especially depending upon the condition of the recipient and the disorder being treated.

Solutions of the active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

As used herein, “pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

The compositions of the present technology may be formulated in a neutral or salt form. Pharmaceutically-acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

The formulations are easily administered in a variety of dosage forms. Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

For oral administration, the polypeptides of the present technology may be incorporated with excipients and used in the form of non-ingestible mouthwashes and dentifrices. It is anticipated that virtually any pill or capsule type known to one of skill in the art including, e.g., coated, and time delay, slow release, etc., may be used with the present technology. A mouthwash may be prepared incorporating the active ingredient in the required amount in an appropriate solvent, such as a sodium borate solution (Dobell's Solution). Alternatively, the active ingredient may be incorporated into an antiseptic wash containing sodium borate, glycerin and potassium bicarbonate. The active ingredient may also be dispersed in dentifrices, including: gels, pastes, creams, powders and slurries. The active ingredient may be added in a therapeutically effective amount to a paste dentifrice that may include water, binders, abrasives, flavoring agents, foaming agents, and humectants.

Pharmaceutical compositions suitable for oral dosage may take various forms, such as tablets, capsules, caplets, and wafers (including rapidly dissolving or effervescing), each containing a predetermined amount of the active agent. The compositions may also be in the form of a powder or granules, a solution or suspension in an aqueous or non-aqueous liquid, and as a liquid emulsion (oil-in-water and water-in-oil). The active agents may also be delivered as a bolus, electuary, or paste. It is generally understood that methods of preparations of the above dosage forms are generally known in the art, and any such method would be suitable for the preparation of the respective dosage forms for use in delivery of the compositions.

In one embodiment, an active agent compound may be administered orally in combination with a pharmaceutically acceptable vehicle such as an inert diluent or an edible carrier. Oral compositions may be enclosed in hard- or soft-shell gelatin capsules, may be compressed into tablets or may be incorporated directly with the food of the patient's diet. The percentage of the composition and preparations may be varied; however, the amount of substance in such therapeutically useful compositions is preferably such that an effective dosage level will be obtained.

Hard capsules containing the active agent compounds may be made using a physiologically degradable composition, such as gelatin. Such hard capsules comprise the compound, and may further comprise additional ingredients including, for example, an inert solid diluent such as calcium carbonate, calcium phosphate, or kaolin. Soft gelatin capsules containing the compound may be made using a physiologically degradable composition, such as gelatin. Such soft capsules comprise the compound, which may be mixed with water or an oil medium such as peanut oil, liquid paraffin, or olive oil.

Sublingual tablets are designed to dissolve very rapidly. Examples of such compositions include ergotamine tartrate, isosorbide dinitrate, and isoproterenol HCl. The compositions of these tablets contain, in addition to the drug, various soluble excipients, such as lactose, powdered sucrose, dextrose, and mannitol. The solid dosage forms of the present technology may optionally be coated, and examples of suitable coating materials include, but are not limited to, cellulose polymers (such as cellulose acetate phthalate, hydroxypropyl cellulose, hydroxypropyl methylcellulose, hydroxypropyl methylcellulose phthalate, and hydroxypropyl methylcellulose acetate succinate), polyvinyl acetate phthalate, acrylic acid polymers and copolymers, and methacrylic resins (such as those commercially available under the trade name EUDRAGIr), zein, shellac, and polysaccharides.

Powdered and granular compositions of a pharmaceutical preparation may be prepared using known methods. Such compositions may be administered directly to a patient or used in the preparation of further dosage forms, such as to form tablets, fill capsules, or prepare an aqueous or oily suspension or solution by addition of an aqueous or oily vehicle thereto. Each of these compositions may further comprise one or more additives, such as dispersing or wetting agents, suspending agents, and preservatives. Additional excipients (e.g., fillers, sweeteners, flavoring, or coloring agents) may also be included in these compositions.

Liquid compositions of pharmaceutical compositions which are suitable for oral administration may be prepared, packaged, and sold either in liquid form or in the form of a dry product intended for reconstitution with water or another suitable vehicle prior to use.

A tablet containing one or more active agent compounds described herein may be manufactured by any standard process readily known to one of skill in the art, such as, for example, by compression or molding, optionally with one or more adjuvant or accessory ingredient. The tablets may optionally be coated or scored and may be formulated so as to provide slow or controlled release of the active agents.

Solid dosage forms may be formulated so as to provide a delayed release of the active agents, such as by application of a coating. Delayed release coatings are known in the art, and dosage forms containing such may be prepared by any known suitable method. Such methods generally include that, after preparation of the solid dosage form (e.g., a tablet or caplet), a delayed release coating composition is applied. Application can be by methods, such as airless spraying, fluidized bed coating, use of a coating pan, or the like. Materials for use as a delayed release coating can be polymeric in nature, such as cellulosic material (e.g., cellulose butyrate phthalate, hydroxypropyl methylcellulose phthalate, and carboxymethyl ethylcellulose), and polymers and copolymers of acrylic acid, methacrylic acid, and esters thereof.

Solid dosage forms according to the present technology may also be sustained release (i.e., releasing the active agents over a prolonged period of time), and may or may not also be delayed release. Sustained release compositions are known in the art and are generally prepared by dispersing a drug within a matrix of a gradually degradable or hydrolyzable material, such as an insoluble plastic, a hydrophilic polymer, or a fatty compound. Alternatively, a solid dosage form may be coated with such a material.

Compositions for parenteral administration include aqueous and non-aqueous sterile injection solutions, which may further contain additional agents, such as antioxidants, buffers, bacteriostats, and solutes, which render the compositions isotonic with the blood of the intended recipient. The compositions may include aqueous and non-aqueous sterile suspensions, which contain suspending agents and thickening agents. Such compositions for parenteral administration may be presented in unit-dose or multi-dose containers, such as, for example, sealed ampoules and vials, and may be stores in a freeze-dried (lyophilized) condition requiring only the addition of the sterile liquid carrier, for example, water (for injection), immediately prior to use. Extemporaneous injection solutions and suspensions may be prepared from sterile powders, granules, and tablets of the kind previously described.

Compositions for rectal delivery include rectal suppositories, creams, ointments, and liquids. Suppositories may be presented as the active agents in combination with a carrier generally known in the art, such as polyethylene glycol. Such dosage forms may be designed to disintegrate rapidly or over an extended period of time, and the time to complete disintegration can range from a short time, such as about 10 minutes, to an extended period of time, such as about 6 hours.

Topical compositions may be in any form suitable and readily known in the art for delivery of active agents to the body surface, including dermally, buccally, and sublingually. Typical examples of topical compositions include ointments, creams, gels, pastes, and solutions. Compositions for administration in the mouth include lozenges.

In accordance with these embodiments, oral (topical, mucosal, and/or dermal) delivery materials can also include creams, salves, ointments, patches, liposomes, nanoparticles, microparticles, timed-release formulations and other materials known in the art for delivery to the oral cavity, mucosa, and/or to the skin of a subject for treatment and/or prevention of a condition disclosed herein. Certain embodiments concern the use of a biodegradable oral (topical, mucosal, and/or dermal) patch delivery system or gelatinous material. These compositions can be a liquid formulation or a pharmaceutically acceptable delivery system treated with a formulation of these compositions, and may also include activator/inducers.

The Smad7 protein fusion constructs of this disclosure, and nucleic acids encoding the same, may be formulated for aerosol administration, particularly to the respiratory tract, including intranasal administration. In order to assure proper particle size in a liquid aerosol, particles can be prepared in respirable size and then incorporated into a colloidial dispersion either containing a propellant as a metered dose inhaler (MDI) or air, such as in the case of a dry powder inhaler (DPI). The active ingredient may be provided in a pressurized pack with a suitable propellant such as a chlorofluorocarbon (CFC) for example dichlorodifluoromethane, trichlorofluoromethane, or dichlorotetrafluoroethane, carbon dioxide or other suitable gas. The dose of drug can be controlled by a metered valve. Alternatively, the active ingredients can be provided in a form of a dry powder, for example a powder mix of the compound in a suitable powder base such as lactose, starch, starch derivatives such as hydroxypropylmethyl cellulose and polyvinylpyrrolidine (PVP). The powder carrier will form a gel in the nasal cavity. The powder composition can be presented in unit dose form for example in capsules or cartridges of, e.g., gelatin or blister packs from which the powder can be administered by means of an inhaler. Alternatively, formulations can be prepared in solution form in order to avoid the concern for proper particle size in the formulation. Solution formulations must nevertheless be dispensed in a manner that produces particles or droplets of respirable size. For MDI application, an aerosol formulation is filled into an aerosol canister equipped with a metered dose valve. In the hands of the patient or subject the formulation is dispensed via an actuator adapted to direct the dose from the valve to the patient or subject.

Generally, formulations for aerosol administration can be prepared by combining (i) the selected drug or drugs in an amount sufficient to provide a plurality of therapeutically effective doses; (ii) the fluid, e.g., propellant, in an amount sufficient to propel a plurality of doses, e.g., from an aerosol canister; (iii) optionally, the water addition in an amount effective to further stabilize each of the formulations; and (iv) any further optional components, e.g., ethanol as a cosolvent; and dispersing the components. The components can be dispersed using a conventional mixer or homogenizer, by shaking, or by ultrasonic energy as well as by the use of a bead mill or a microfluidizer. Bulk formulations can be transferred to smaller individual aerosol vials by using valve to valve transfer methods, pressure filling or by using conventional cold-fill methods. It is not required that a component used in a suspension aerosol formulation be soluble in the fluid carrier, e.g., propellant. Components that are not sufficiently soluble can be coated or congealed with polymeric, dissolution controlling agents in an appropriate amount and the coated particles can then be incorporated in a formulation as described above. Polymeric dissolution controlling agents suitable for use in this invention include, but not limited to polylactide glycolide co-polymer, acrylic esters, polyamidoamines, substituted or unsubstituted cellulose derivatives, and other naturally derived carbohydrate and polysaccharide products such as zein and chitosan.

The compositions for use in the methods of the present technology may also be administered transdermally, wherein the active agents are incorporated into a laminated structure (generally referred to as a “patch”) that is adapted to remain in intimate contact with the epidermis of the recipient for a prolonged period of time. Typically, such patches are available as single layer “drug-in-adhesive” patches or as multi-layer patches where the active agents are contained in a layer separate from the adhesive layer. Both types of patches also generally contain a backing layer and a liner that is removed prior to attachment to the recipient's skin. Transdermal drug delivery patches may also be comprised of a reservoir underlying the backing layer that is separated from the skin of the recipient by a semi-permeable membrane and adhesive layer. Transdermal drug delivery may occur through passive diffusion, electrotransport, or iontophoresis.

In certain embodiments, a patch contemplated herein may be a slowly dissolving or a time-released patch. In accordance with these embodiments, a slowly dissolving patch can be an alginate patch. In certain examples, a patch may contain a detectible indicator dye or agent such as a fluorescent agent. In other embodiments, a tag (e.g., detectible tag such as a biotin or fluorescently tagged agent) can be associated with a treatment molecule in order to detect the molecule after delivery to the subject. In certain embodiments, one or more oral delivery patches or other treatment contemplated herein may be administered to a subject three times daily, twice daily, once a day, every other day, weekly, and the like, depending on the need of the subject as assessed by a health professional. Patches contemplated herein may be oral-biodegradable patches or patches for exterior use that may or may not degrade. Patches contemplated herein may be 1 mm, 2 mm, 3 mm, 4 mm to 5 mm in size or more depending on need. In addition, skin patches are contemplated herein for use for example in a subject suffering from psoriasis. In treating psoriasis and chronic wounds, Smad7 can be delivered topically using vehicles such as glycerol, carboxymethycellulose. It can also use transdermal system (e.g., commercially available from 3M) for delivery. Subcutaneous injection into the lesion (in normal saline or PBS) can also be used.

In some embodiments, compositions may include short-term, rapid-onset, rapid-offset, controlled release, sustained release, delayed release, and pulsatile release compositions, providing the compositions achieve administration of a compound as described herein. See Remington's Pharmaceutical Sciences (18th ed.; Mack Publishing Company, Eaton, Pa., 1990), herein incorporated by reference in its entirety.

In certain embodiments, the compounds and compositions disclosed herein can be delivered via a medical device. Such delivery can generally be via any insertable or implantable medical device, including, but not limited to stents, catheters, balloon catheters, shunts, or coils. In one embodiment, the present technology provides medical devices, such as stents, the surface of which is coated with a compound or composition as described herein. The medical device of this technology can be used, for example, in any application for treating, preventing, or otherwise affecting the course of a disease or condition, such as those disclosed herein.

It is contemplated that any molecular biology, cellular biology or biochemical technique known in the art may be used to generate and/or test treatments provided herein. In addition, protein chemistry techniques are contemplated to assess utility of treatments in model systems developed herein (e.g., mouse model system).

11. Combination Therapies

It is common in many fields of medicine to treat a disease with multiple therapeutic modalities, often called “combination therapies.” Many of the diseases described herein (e.g., inflammatory disease and cancer) are no exception. In some embodiments, to treat inflammatory disorders using the methods and compositions of the present technology, one would contact a target cell, organ or subject with a Smad7 protein, expression construct or activator and at least one other therapy. These therapies would be provided in a combined amount effective to achieve a reduction in one or more disease parameter. This process may involve contacting the cells/subjects with the both agents/therapies at the same time, e.g., using a single composition or pharmacological formulation that includes both agents, or by contacting the cell/subject with two distinct compositions or formulations, at the same time, wherein one composition includes the Smad7 agent and the other includes the other agent.

Alternatively, the Smad7 agent may precede or follow the other treatment by intervals ranging from minutes to weeks. One would generally ensure that a significant period of time did not expire between the time of each delivery, such that the therapies would still be able to exert an advantageously combined effect on the cell/subject. In such instances, it is contemplated that one would contact the cell with both modalities within about 12-24 hours of each other, within about 6-12 hours of each other, or with a delay time of only about 12 hours. In some situations, it may be desirable to extend the time period for treatment significantly; however, where several days (2, 3, 4, 5, 6 or 7) to several weeks (1, 2, 3, 4, 5, 6, 7 or 8) lapse between the respective administrations.

It also is conceivable that more than one administration of either the Smad7 agent or the other therapy will be desired. Various combinations may be employed, where the Smad7 agent is “A,” and the other therapy is “B,” as exemplified below:

| | | | |
|---|---|---|---|
| A/B/A | B/A/B | B/B/A | A/A/B |
| B/A/A | A/B/B | B/B/B/A | B/B/A/B |
| A/A/B/B | A/B/A/B | A/B/B/A | B/B/A/A |
| B/A/B/A | B/A/A/B | B/B/B/A | A/A/A/B |
| B/A/A/A | A/B/A/A | A/A/B/A | A/B/B/B |
| B/A/B/B | B/B/A/B | | |

Other combinations are provided. Other agents suitable for use in a combined therapy against an inflammatory disorder include steroids, glucocorticoids, non-steriodal anti-inflammatory drugs (NSAIDS; including COX-1 and COX-2 inhibitors), aspirin, ibuprofen, and naproxen. Analgesics are commonly associated with anti-inflammatory drugs but which have no anti-inflammatory effects. An example is paracetamol, called acetaminophen in the U.S. and sold under the brand name of Tylenol. As opposed to NSAIDS, which reduce pain and inflammation by inhibiting COX enzymes, paracetamol has recently been shown to block the reuptake of endocannabinoids, which only reduces pain, likely explaining why it has minimal effect on inflammation. A particular agent for combination use is an anti-TGF-β antibody.

The skilled artisan is directed to Remington's Pharmaceutical Sciences, 15th Edition, chapter 33, in particular, pages 624-652, 1990. Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

It also should be pointed out that any of the foregoing therapies may prove useful by themselves in treating inflammation.

As discussed above, the present technology has particular relevance to the treatment of DNA damage and/or inflammation resulting from certain anti-cancer therapies, and for the treatment of cancer. Thus, in particular, the present technology may be applied as a combination with cancer therapies. This process may involve contacting the cells, organ, or patient with the agents/therapies at the same time, including by contacting the cells, organ or patient with a single composition or pharmacological formulation that includes both agents, or with two distinct compositions or formulations at the same time, wherein one composition includes the Smad7 agent and the other includes the other agent. Alternatively, analogous to the chart set forth above, the compositions can be delivered at different times, including repeated doses of one or both agents.

Agents or factors suitable for use in a combined therapy include any chemical compound or treatment method that induces DNA damage when applied to a cell. Such agents and factors include radiation and waves that induce DNA damage such as, irradiation, microwaves, electronic emissions, and the like. A variety of chemical compounds, also described as “chemotherapeutic” or “genotoxic agents,” are intended to be of use in the combined treatment methods disclosed herein. In treating cancer according to the present technology, one would contact the tumor cells with an agent in addition to the expression construct. This may be achieved by irradiating the localized tumor site; alternatively, the tumor cells may be contacted with the agent by administering to the subject a therapeutically effective amount of a pharmaceutical composition.

Various classes of chemotherapeutic agents are provided for use with in combination with peptides of the present technology. For example, selective estrogen receptor antagonists (“SERMs”), such as Tamoxifen, 4-hydroxy Tamoxifen (Afimoxfene), Falsodex, Raloxifene, Bazedoxifene, Clomifene, Femarelle, Lasofoxifene, Ormeloxifene, and Toremifene.

Chemotherapeutic agents contemplated to be of use, include, e.g., camptothecin, actinomycin-D, mitomycin C. The present technology also encompasses the use of a combination of one or more DNA damaging agents, whether radiation-based or actual compounds, such as the use of X-rays with cisplatin or the use of cisplatin with etoposide. The agent may be prepared and used as a combined therapeutic composition, or kit, by combining it with a MUC1 peptide, as described above.

Heat shock protein 90 is a regulatory protein found in many eukaryotic cells. HSP90 inhibitors have been shown to be useful in the treatment of cancer. Such inhibitors include Geldanamycin, 17-(Allylamino)-17-demethoxygeldanamycin, PU-H71 and Rifabutin.

Agents that directly cross-link DNA or form adducts are also envisaged. Agents such as cisplatin, and other DNA alkylating agents may be used. Cisplatin has been widely used to treat cancer, with efficacious doses used in clinical applications of 20 mg/m² for 5 days every three weeks for a total of three courses. Cisplatin is not absorbed orally and must therefore be delivered via injection intravenously, subcutaneously, intratumorally or intraperitoneally.

Agents that damage DNA also include compounds that interfere with DNA replication, mitosis and chromosomal segregation. Such chemotherapeutic compounds include adriamycin, also known as doxorubicin, etoposide, verapamil, podophyllotoxin, and the like. Widely used in a clinical setting for the treatment of neoplasms, these compounds are administered through bolus injections intravenously at doses ranging from 25-75 mg/m² at 21-day intervals for doxorubicin, to 35-50 mg/m² for etoposide intravenously or double the intravenous dose orally. Microtubule inhibitors, such as taxanes, also are contemplated. These molecules are diterpenes produced by the plants of the genus Taxus, and include paclitaxel and docetaxel.

Epidermal growth factor receptor inhibitors, such as Iressa, mTOR, the mammalian target of rapamycin, also known as FK506-binding protein 12-rapamycin associated protein 1 (FRAP1) is a serine/threonine protein kinase that regulates cell growth, cell proliferation, cell motility, cell survival, protein synthesis, and transcription. Rapamycin and analogs thereof (“rapalogs”) are therefore provided for use in combination cancer therapy in accordance with the present technology.

Another possible combination therapy with the peptides claimed herein is TNF-α (tumor necrosis factor-alpha), a cytokine involved in systemic inflammation and a member of a group of cytokines that stimulate the acute phase reaction. The primary role of TNF is in the regulation of immune cells. TNF is also able to induce apoptotic cell death, to induce inflammation, and to inhibit tumorigenesis and viral replication.

Agents that disrupt the synthesis and fidelity of nucleic acid precursors and subunits also lead to DNA damage. As such a number of nucleic acid precursors have been developed. Particularly useful are agents that have undergone extensive testing and are readily available. As such, agents such as 5-fluorouracil (5-FU), are preferentially used by neoplastic tissue, making this agent particularly useful for targeting to neoplastic cells. Although quite toxic, 5-FU, is applicable in a wide range of carriers, including topical, however intravenous administration with doses ranging from 3 to 15 mg/kg/day being commonly used.

Other factors that cause DNA damage and have been used extensively include what are commonly known as γ-rays, x-rays, and/or the directed delivery of radioisotopes to tumor cells. Other forms of DNA damaging factors are also contemplated such as microwaves and UV-irradiation. It is most likely that all of these factors effect a broad range of damage DNA, on the precursors of DNA, the replication and repair of DNA, and the assembly and maintenance of chromosomes. Dosage ranges for x-rays range from daily doses of 50 to 200 roentgens for prolonged periods of time (3 to 4 weeks), to single doses of 2000 to 6000 roentgens. Dosage ranges for radioisotopes vary widely, and depend on the half-life of the isotope, the strength and type of radiation emitted, and the uptake by the neoplastic cells.

The skilled artisan is directed to Remington's Pharmaceutical Sciences, 15th Edition, chapter 33, in particular pages 624-652. Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

In addition to combining Smad7 therapies with chemo- and radiotherapies, it also is contemplated that combination with immunotherapy, hormone therapy, toxin therapy and surgery. In particular, one may employ targeted therapies such as AVASTIN®, ERBITUX®, GLEEVEC®, HERCEPTIN®, and RITUXAN®.

In other embodiments, to assess the roles and mechanisms of Smad7 within the context of oral mucositis, “gene-switch” transgenic mouse models were developed to allow control of the level and duration of Smad7 transgene expression specifically in oral epithelia. In accordance with these embodiments, these models may be used to test other genes or downstream molecules for their effects on oral epithelia and oral mucosa. Thus, these models can be used for, but are not limited to, further analysis of oral wound healing biology and testing therapeutic approaches to oral wound healing. Molecular Smad7 targets identified in these studies can provide additional therapeutic targets for subjects suffering from oral mucositis. Models and resources developed herein can provide unique tools for analytical studies to identify biomarkers and therapeutic targets related to Smad7 overexpression and control, for example, downstream molecules turned on or bound by Smad7 can be identified as additional therapeutic targets for example, to treat oral mucositis, psoriasis and other conditions aggravated by TGF-β activities and NF-κB activities.

### D. Kits

In certain embodiments, a kit provided herein may include compositions discussed above for treating a subject having a condition provided herein, such as but not limited to oral mucositis, psoriasis, or wound healing. The kits can include one or more containers containing the therapeutic Smad7 compositions of the present technology. Any of the kits will generally include at least one vial, test tube, flask, bottle, syringe or other container, into which compositions may be preferably and/or suitably aliquoted. Kits herein may also include a kit for assessing biological targets that contribute to a condition provided herein.

### E. Methods of Predicting or Evaluating Responses

Also provided are methods for predicting and/or evaluating a response to treatment with Smad7 using by assessing the level of expression of one or more markers associated with exposure to Smad7. Such markers may include, but are not limited to, Rac1 for cell migration, NF-κB for inflammation, and TGF-β for growth arrest and inflammation. As is discussed in the Examples, methods for detection of and/or changes in the levels of one or more markers associated with Smad7 activity are provided and/or known in the art. In some embodiments, the level of expression of one or more of the Smad7 markers in a subject may be assessed, and based on the level detected, a decision may be made to treat (or to continue or discontinue treatment) with Smad7, or to employ an alternate treatment.

The term “detection of” as used herein refers to the ability to measure the presence or absence of a marker at some repeatable and controlled level. Typically, detection is performed over background values, which may include the noise (or detection limits) inherent in the testing system. As such, there is typically a “lower limit” of detection associated with an assay, and in order to be detected, a change may need to be above a certain cut-off level, for example. Determination of such limits is well-known in the art.

In some embodiments, detection is performed as compared to controls, which may include, but are not limited to, a comparison with data from normal subjects and/or comparable normal tissue (in the same or different subjects) absent the disease or disorder present in the subject (or the specific tissue of the subject tested). In some embodiments, the comparison may be between levels detected at a variety of time intervals (and/or locations) in a patient. In some embodiments, the detection needs to be statistically significant as compared to background or control levels; the ability to assess significance is well-known in the art, and exemplified in the Examples.

The term “changes in the levels” as used herein refers to a detectable change from a control or background level, and or a previously detected level. In some embodiments, the change is an increase as compared to another level, and in some embodiments the change is a decrease as compared to another level. In some embodiments, the detectable change (increase or decrease) is statistically significant. In some embodiments, such changes can be assessed quantitatively as at least about a 5%, 10%, 25%, 50%, 100%, 200%, 500% or greater change, and/or about a 5-10%, 10-25%, 10-50%, 25-50%, 50-75%, 50-100%, 100-150%, 100-200%, 200-300%, 300-500%, or 500-1000% change.

### F. Method of Screening for Additional Biologically Active Fragments

In another aspect, methods of screening for additional biologically active fragments (including, but not limited to truncations) of Smad7 are contemplated. In some embodiments, biological activity may be assessed using one of the methods described herein, including those described below. Some of the biological activities that can be assessed include, but are not limited to, increasing cell proliferation, reducing or inhibiting cell death, reducing excessive inflammation, preventing DNA damage, and/or increasing cell migration, as well as animal models treating or preventing one or more disease or disorders in which such treatment would be helpful as further discussed herein. Such activities can be assessed using one or more assays including, but not limited to, the ability to block phosphorylation of Smad2 and/or nuclear translocation of the NF-κB p50 subunit, increase cell proliferation, reduce apoptosis and/or radiation-induced DNA damage, reduce inflammation and/or angiogenesis, promote healing in oral mucositis, surgical wounds, diabetes wounds, and/or wounds associated with chronic inflammation in mice and other laboratory models. Some specific examples include, but are not limited to, immunofluorescence (IF), immunohistochemistry (IHC), and TUNEL assay for apoptosis.

In some embodiments, biologically active fragments are those that are selected to include one or more or all of the activities described herein. In some embodiments, biologically active fragments selected to include only or primarily 1, only or primarily 2, only or primarily 3, only or primarily 4, or only or primarily 5 of the activities described herein. In some embodiments, biologically active fragments selected to exclude only or primarily 1, only or primarily 2, only or primarily 3, only or primarily 4, or only or primarily 5 of the activities described herein. In some embodiments, the biologically active fragments are selected to include or to exclude a specific subset of the activities described herein. For instance, increased proliferation and migration may be sufficient for treating diabetic wounds, whereas anti-inflammation is needed in chronic inflammatory wounds. Reduced apoptosis and DNA damage activities are needed for treating oral mucositis but not for treating surgical wounds

The term “primarily includes” as used herein refers to fragments in which although some level of other biological activity may remain, that activity is reduced as compared with full-length fragments, whereas the activity that is considered “primary” remains at about the same or an increased level as that observed in the full-length native protein. Similarly, the term “primarily excludes” as used herein refers to fragments in which although some level of a particular biological activity may remain, the level of that particular activity is reduced (optionally significantly and/or statistically significantly reduced) as compared with full-length fragments, whereas one or more other biological activities remains at about the same or increased level as that observed in the full-length native protein.

In some embodiments involving selection of biologically active fragments, the methods include assessing changes in the level of expression of one or more biological activities, including increases and decreases of one or more activities in a selected fragment are assessed as changes in reference to the activities observed in the full-length protein. In some embodiments, one or more biological activities are being selected to remain the same as that observed in the full-length fragments while other activities may be increased or decreased or even eliminated (e.g., such fragments would lack one or more of the activities discussed). In some embodiments, the change is an increase as compared to another level, and in some embodiments the change is a decrease as compared to another level. In some embodiments, the detectable change (increase or decrease) is statistically significant. In some embodiments, such changes can be assessed quantitatively as at least about a 5%, 10%, 25%, 50%, 100%, 200%, 500% or greater change, and/or about a 5-10%, 10-25%, 10-50%, 25-50%, 50-75%, 50-100%, 100-150%, 100-200%, 200-300%, 300-500%, or 500-1000% change. In some embodiments, an activity that “remains the same” can still be observed to have some change from the activity of the full-length protein, but such change might be limited to, for example, about a 1%, 2%, 5%, 10%, or 20% change or less.

In a non-limiting example, fragments of interest may include those that primarily mediate the anti-inflammatory effect of Smad7. Smad7 peptides having this anti-inflammatory function may be sufficient and optionally an improvement for treating chronic inflammation associated conditions, such as but not limited to, oral mucositis, stomatitis and psoriasis, among others. In another non-limiting example, fragments of interest may include those that primarily mediate cell migration and/or blocking TGF-β-induced growth arrest and/or fibrotic response. Smad7 peptides having this cell migration and proliferation function may be sufficient, and optionally an improvement, for enhancing healing that is not associated with excessive inflammation. Types of wounds that might benefit from this form of treatment include, but are not limited to, surgical wounds, fibrotic scarring, and diabetes wounds, defective healing and/or scarring (including keloid and hypertrophic scarring) among others.

### G. Methods of Producing Smad7 Protein

In another aspect, methods for producing Smad7 protein, including any of the Smad7 variants, fragments, truncations, fusion proteins (e.g., PTD-Smad7) described herein are contemplated. The inventor has discovered methods of producing Smad7 protein at levels and purity sufficient for research, development, or commercialization that include nucleic acid codon optimization. As a result, methods for producing Smad7 including the use of one or more of the codon-optimized Smad7 nucleic acid molecules described herein (e.g., within the Examples) are expressly contemplated.

### EXAMPLES

The following examples are included to illustrate various embodiments. It should be appreciated by those of skill in the art that the techniques disclosed in the examples that follow represent techniques discovered to function well in the practice of the claimed methods, compositions and apparatus. However, those of skill in the art should, in light of the present disclosure, appreciate that many changes may be made in the specific embodiments which are disclosed and still obtain a like or similar result without departing from the spirit and scope of the present technology.

The inventor previously developed a biologic containing human Smad7 fused to a small peptide from the HIV-1 Tat protein that rapidly penetrates cells upon contact (Han G, Bian L, Li F, Cotrim A, Wang D, Lu J, et al. Preventive and therapeutic effects of Smad7 on radiation-induced oral mucositis. Nat Med. 2013; 19(4):421-8; U.S. patent application Ser. No. 14/773,167, Publication No. US 2016/0039894, which is incorporated herein in its entirety). The inventor has since made several Tat-Smad7 truncated variants (U.S. patent application Ser. No. 15/208,424, U.S. Pub. No. 2017/0000851), and shown that they generally retain some, but not all, of the activities of the full-length Tat-Smad7 construct. In addition, the activity is typically lower than that of the full-length Tat-Smad7 construct as might be expected, presumably because of changes in tertiary structure, degradation properties, or lack of interplay with other portions of the protein, such as sequences providing the ability to traffic between the nucleus and the cytoplasm. FIG. 1B summarizes potential molecular mechanisms of the multifunctional nature of Tat-PY-C-Smad7.

In contrast, the inventor has surprisingly discovered that Tat-PY-C-Smad7 containing amino acids 203-426aa of human Smad7, has substantially higher biological acitivities than the Tat-Smad7 fragment constructs described in US 2016/0039894. This new construct, Tat-PY-C-Smad7, also unexpectedly has activities similar to, or better than, full length Tat-Smad7 in multiple medical indications. This discovery was completely unexpected as none of the inventor's previous data provided any indication that the amino acids 203-426aa of human Smad7 fragment, linked in this fusion construct would have significantly higher, or even significantly different, therapeutic activities compared to the full length human Smad7-Tat fusion construct.

### Example 1: Tat-PY-C-Smad7 Fusion Protein Shows Activity in Oral Mucositis

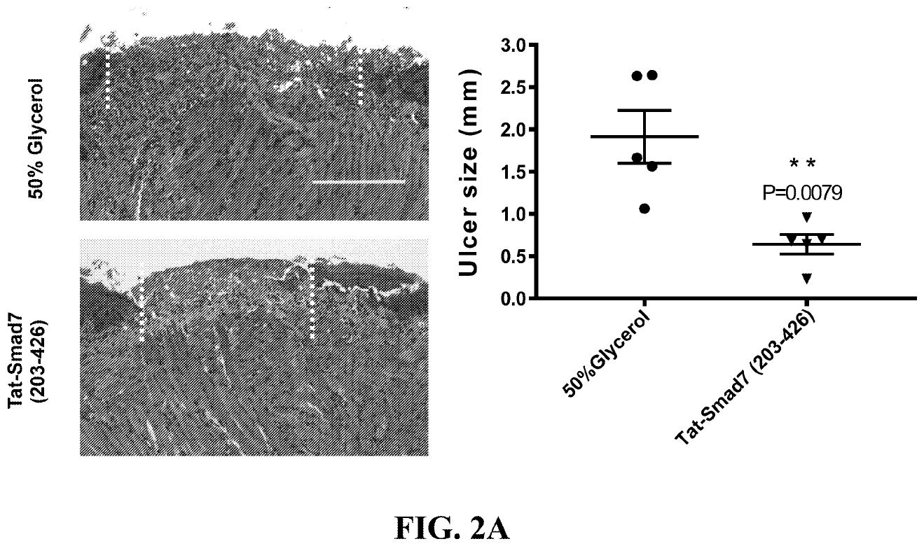

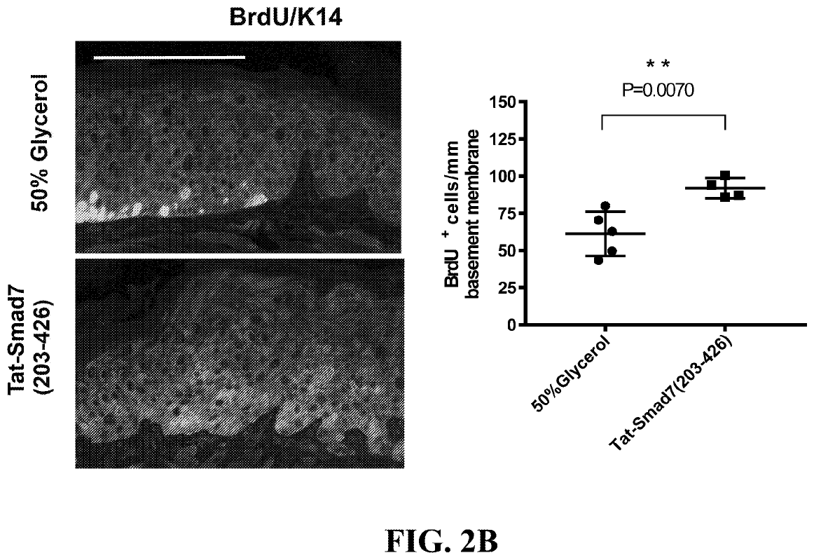

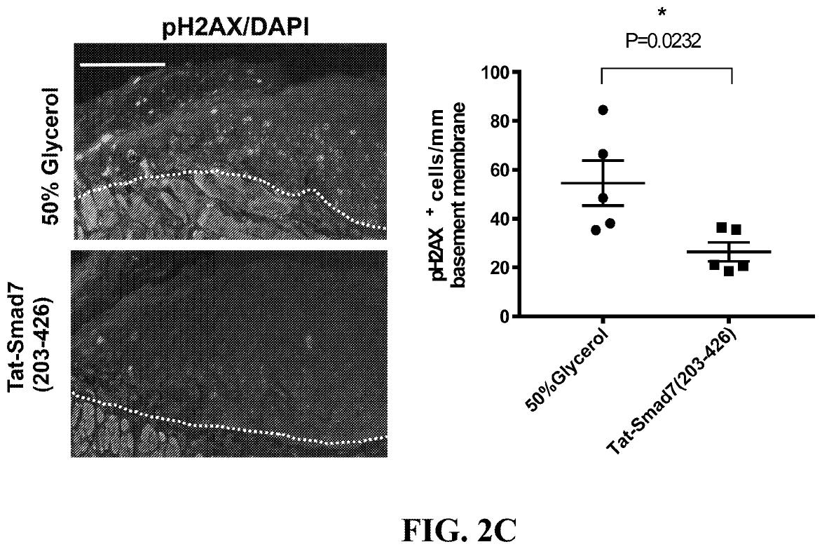

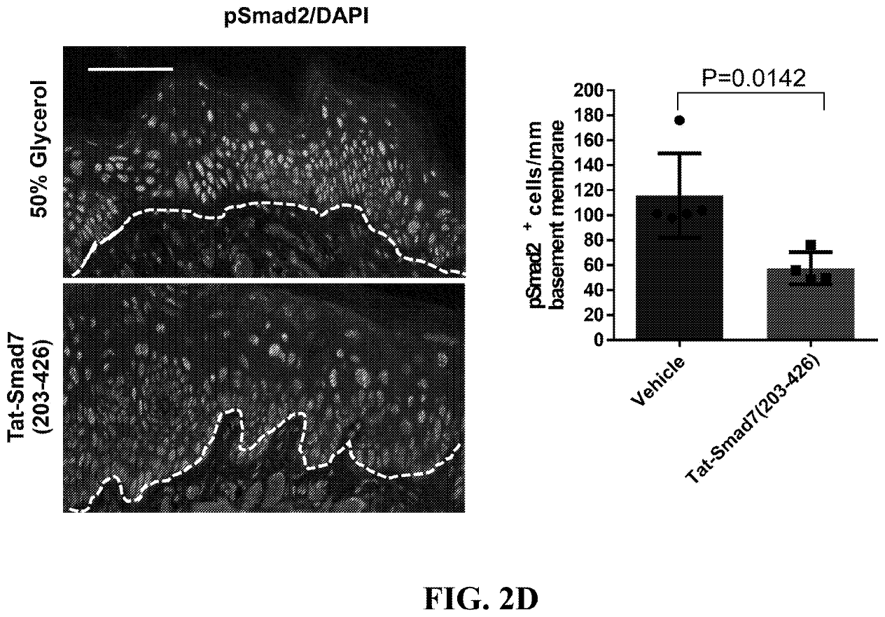

The therapeutic potential of the Tat-PY-C-Smad7 fusion protein was tested initially in radiation-induced oral mucositis. Tat-PY-C-Smad7 (1 μg/mouse) was applied directly to the mouse oral cavity daily starting day 6 after craniofacial radiation (18Gy) when irradiated oral mucosa began to develop oral mucositis (Han G, et al., Nat Med. 2013; 19(4):421-8, supra). On day 10, oral tissues were taken for pathological evaluation. Histology shows that the vehicle group had ulcerations with massive numbers of infiltrated inflammatory cells (FIG. 2A, left panel). Tat-PY-C-Smad7 treatment significantly reduced ulcer sizes and inflammation (FIG. 2A, right panel). Further, Tat-PY-C-Smad7 increased proliferation (BrdU+ cells) and decreased cells positive for phospho-H2AX (pH2AX)+ foci (DNA damage) in mucosa adjacent to ulcers (FIGS. 2B, 2C). Tat-PY-C-Smad7 also decreased phosphorylated Smad2 (pSmad2), which is a receptor-activated Smad downstream of TGF-β signaling that is translocated into the nucleus to modulate the transcription of target genes contributing to oral mucositis development (FIG. 2D).

### Example 2: Tat-PY-C-Smad7 Fusion Protein Shows Activity in Radiation-Induced Dermatitis

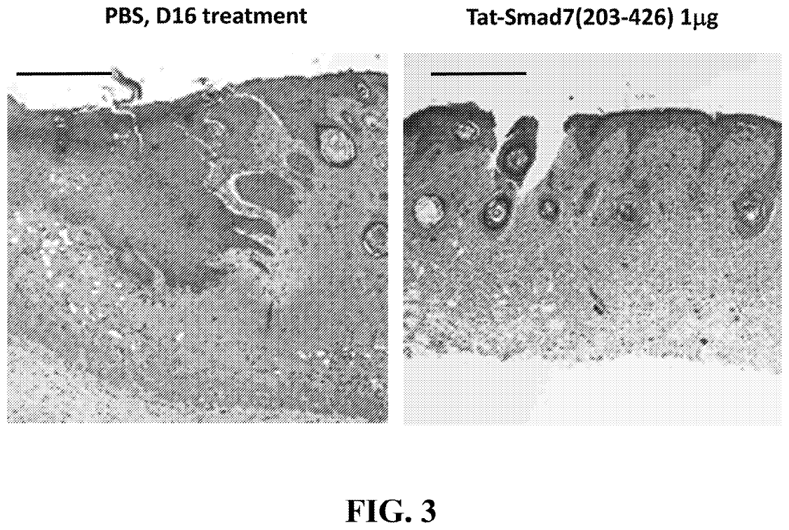

The inventor also tested Tat-PY-C-Smad7 treatment in radiation-induced dermatitis in mice. Mouse legs were irradiated with 35Gy. By day 11, when the skin started to develop dermatitis, Tat-PY-C-Smad7 (1 μg/mouse) or PBS (vehicle) was injected into lesional skin every other day until day 27. Although the mechanisms of radiation induced tissue damage are similar between oral mucositis and dermatitis, the latter also has epidermal hyperplasia/proliferation induced by massive inflammation as well as ulcers and significant fibrosis evidenced in skin from vehicle-treated control mice (FIG. 3A, left panel). These pathological characteristics were alleviated by Tat-PY-C-Smad7 treatment (FIG. 3B, right panel).

### Example 3: Tat-PY-C-Smad7 Fusion Protein Shows Activity in Psoriasis, Atopic Dermatitis and Inhibiting Fibrosis

The inventor previously generated K5.TGFβ1 transgenic mice in either C57BL/6 or ICR background with severe skin psoriasis. These mice die within six (6) months due to a severe itch-associated wasting syndrome (Li A G, Wang D, Feng X H, Wang X J. Latent TGFbetal overexpression in keratinocytes results in a severe psoriasis-like skin disorder. EMBO J. 2004; 23(8):1770-81). Tat-PY-C-Smad7 was injected subcutaneously (s.c.) (2 μg/mouse, 3 times/week) into K5.TGFβ1 lesional skin. Tat-PY-C-Smad7 treatment healed the lesions by gross inspection by Day 10 (FIG. 4A), while untreated lesions continue to worsen. Tat-PY-C-Smad7 significantly alleviated skin inflammation as early as 6 days after treatment, with histology showing that treatment reduced epidermal hyperplasia, inflammation and fibrotic response as early as 13 days after treatment (FIG. 4B). No obvious toxic effects were observed.

Tat-Smad7(203-426)'s therapeutic effect is much faster and better than full length Tat-Smad7 shown in a recent publication (Li et al., Journal of Investigative Dermatology, doi: 10.1016/j.jid.2018.10.031. [Epub ahead of print] PMID: 30423327). That study, led by the inventor, shows that it takes 18 days for full length Tat-Smad7 to show gross alleviation of skin inflammation in K5.TGFβ1 skin and 28 days to completely reverse skin inflammation in this model.

These K5.TGFβ1 transgenic mice can also serve as a model for contact dermatitis or atopic dermatitis when they show, for example, the presence of a mast cell infiltrate in the woundsas as in FIG. 4C (left panel). Tat-Smad7(203-426) was found to have reduced the number of mast cells by Day 13 (right panel), indicating its utility in treating contact dermatitis or atopic dermatitis.

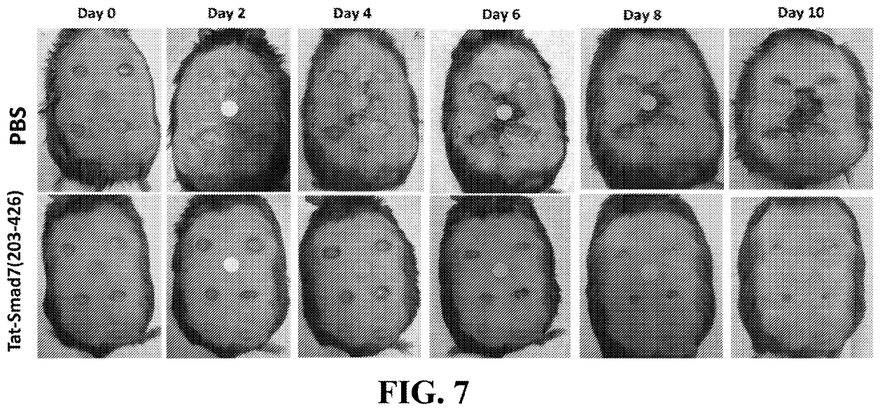

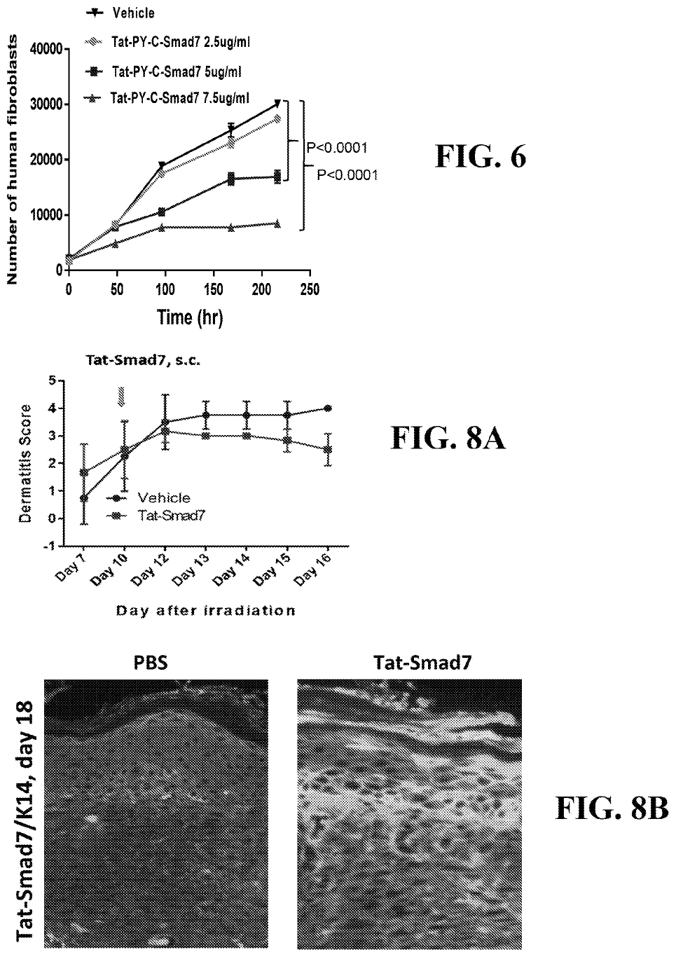

The inventor also studied the activity of Tat-PY-C-Smad7 fusion protein on fibroblast proliferation in vitro. Primary human skin fibroblasts were cultured with Tat-Smad7(203-426) at 2.5 μg/ml, 5.0 μg/ml, and 7.5 μg/ml or with vehicle (PBS) alone for about 9 days. Tat-PY-C-Smad7 at 5 μg/ml and 7.5 μg/ml significantly reduced fibroblast expansion compared with vehicle-treated controls, consistent with Tat-PY-C-Smad7's anti-fibroblast activation and anti-fibrosis effect in vivo (FIG. 6).

### Example 4: Tat-PY-C-Smad7 Fusion Protein Shows Activity in Diabetic Wounds

Tat-Smad7(203-426) activity was assessed in a mouse model of diabetic (db/db) wound healing. Tat-Smad7(203-426) was applied topically, 1 μg/wound, every other day beginning immediately after punch biopsies to create the wounds (day 0). There was no observed healing by day 10 in PBS treated wounds (controls), but in contrast Tat-Smad7(203-426) treated wounds had noticeable healing beginning on day 8 and most of them were healed by day 10 (FIG. 7A). This result was supported by histology of the H&E stained skin sections that showed unhealed wound morphology in PBS treated wound and healed wound treated by Tat-Smad7(203-426). Unexpectedly, Tat-Smad7(203-426) also performed significantly better than Regranax™, an FDA-approved drug for treating diabetic foot wounds, in a 12-day comparison as summarized in the following table that summarizes wound healing rates in diabetic mice treated with Vehicle control (PBS), Regranex™, applied topically every other day, and Tat-Smad7(203-426) applied topically every other day. The difference between the numbers of healed mice in the treated and control (P=0.03, Day 10; P=0.02, Day 12) and Regranax groups (p=0.014, Day 12) was significant.

| | Healed/Total wounds | Healed/Total wounds |
|---|---|---|
| Group | (day 10) | (day 12) |
| PBS | 0/4 | 1/6 |
| Regranex | NA | 0/4 |
| Tat-Smad7 | 6/8 P = 0.03 | 12/16 |
| (203-426) | | (p = 0.014 vs. Regranex) |
| | | (p = 0.02 vs. PBS) |

Tat-Smad7(203-426) also performed better than full-length Tat-Smad7, the effect of the latter was obvious after 10 days of treatment and completed healing by day 13 (see, WIPO patent application No. PCT/US2014/022052, published as WO/2014/138670, filed 7 Mar. 2014, which is incorporated by reference in its entirety).

### Example 5: Tat-Smad7 Shows Activity in Radiation-Induced Dermatitis

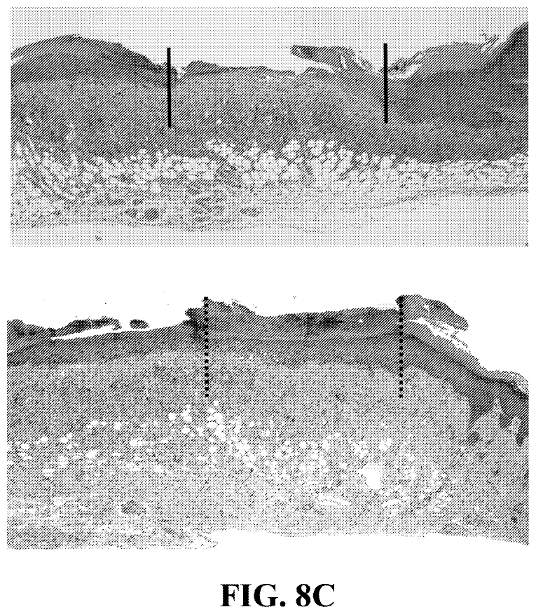

The treatment effect of full lengthTat-Smad7 on radiation induced dermatitis was assessed in a mouse model. As described above, dermatitis was induced by 35Gy radiation to the legs of mice. Tat-Smad7, 1 μg/mouse, was injected subcutaneously 3 times/week, starting on day 7 after radiation. The severity of the dermatitis (Dermatitis Score), which was assessed daily using scoring criteria established by the Cancer Therapy Evaluation Program, showed that by Day 18 the Tat-Smad7-treated mice had significantly less severe dermatitis. *: p<0.05 (FIG. 8A). Skin biopsies harvested on day 18 were assessed for the presence of Tat-Smad7 (FIG. 8B). Immunofluorescence staining using an antibody specific for Tat-Smad7 shows that Tat-Smad7 (light gray) penetrated into both dermal cells and epidermal cells. K14 antibody staining (dark gray) highlights the epidermis. The skin biopsies were also stained with H&E for histology which shows that treated skin had healed ulcers, and reductions in inflammation and in fibrotic response (FIG. 8C). Epidermal hyperplasia adjacent to ulcers, as a result of inflammation, was also reduced by treatment. Vertical lines highlight unhealed ulcer in PBS control. Dotted lines highlight healed ulcer treated by Tat-Smad7.

### Example 6: Tat-Smad7 Shows Activity in a Model of Atopic Dermatitis

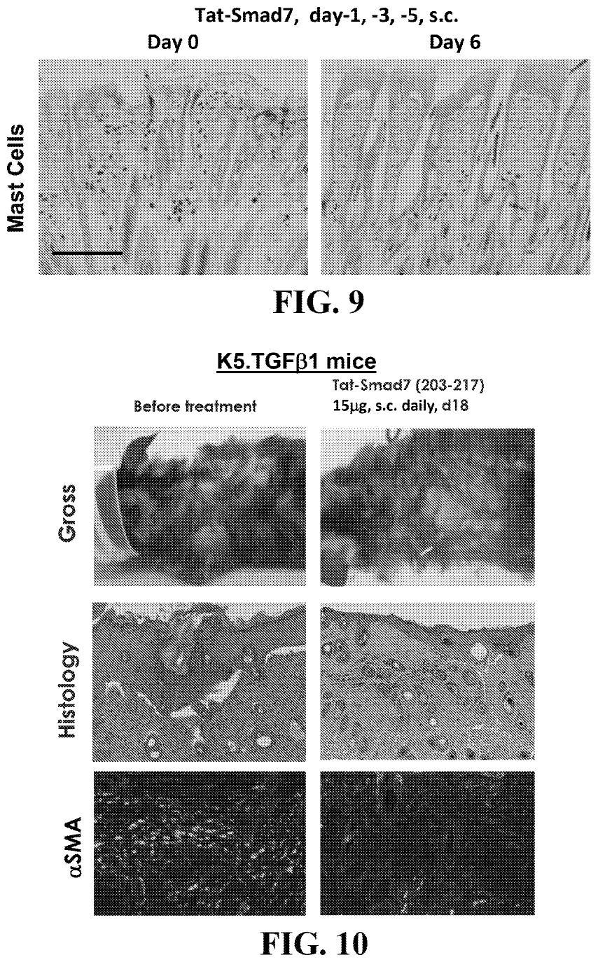

As discussed briefly above, in some instances a mouse model for psoriasis, K5.TGFβ1 mice, can also serve as a model for contact dermatitis or atopic dermatitis in the presence of exogenous pathogens. Mast cells are a hallmark of this disease, as they are the major pathogenic cells for contact/allergic dermatitis. Full length Tat-Smad7 reduced the number of mast cells prior to complete reversal of skin inflammation in this model. K5.TGFβ1 skin was treated by Tat-Smad7, subcutaneously (s.c.) 1 μg/mouse, 3×/wk. Skin biopsies were taken, sectioned, and stained mast cells with Giemsa or Toluidine blue prior to treatment (day 0) and on day 6 of Tat-Smad7 treatment, prior to its reversal of epidermal hyperplasia and inflammation (FIG. 9). The decrease in the number of mast cells in the Tat-Smad7 treated sections indicates that mast cells are a direct therapeutic target of Tat-Smad7.

### Example 7: Tat-Smad7(203-217) Shows Activity Treating Skin Inflammation and Fibrosis

FIG. 10 shows that Tat-Smad7(203-217) alleviated TGFβ1-induced skin inflammation and fibrosis. Tat-Smad7(203-217) peptide was injected s.c. daily, 15 μg in phosphate buffered saline (PBS), into the skin of K5.TGFβ1 mice. Untreated K5.TGFβ1 skin progressively deteriorates, and treatment with vehicle (PBS) alone does not alleviate the phenotype. Skin biopsies were taken before (Day 0) and after treatment (Day 18). Inflamed skin gradually improved macroscopically beginning 2 weeks after treatment and was grossly obvious by 3 weeks. Similar results were seen by histology, where peptide-treated skin showed reductions in leukocyte infiltration and epidermal hyperplasia and reduced alpha-smooth muscle actin (αSMA), a marker of activated fibroblasts.

### Example 8: Tat-Smad7(203-217) Shows Activity in Radiation-Induced Dermatitis

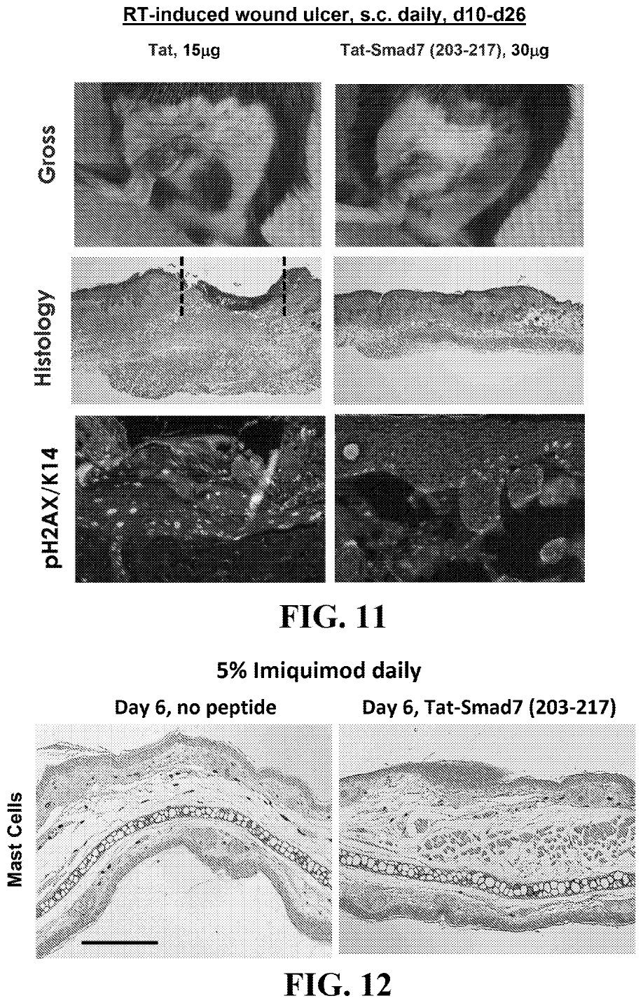

FIG. 11 shows that Tat-Smad7(203-217) promoted healing of RT-induced would ulcer in an experimental mouse model. The leg skin was shaved and received 30Gy radiation. By 10 days after RT, irradiated skin was inflamed and wound ulcers began to develop. Skin wounds were treated with daily subcutaneous injections of either 15 μg Tat (control peptide) or 30 μs Tat-Smad7 (203-217) peptide to the irradiated skin. Cells with nuclear pH2AX, a DNA damage marker, were fewer in treated skin, and lower in skin sections from mice treated with Tat-Smad7 (203-217) compared with sections from mice treated with control peptide.

### Example 9: Tat-Smad7(203-217) Shows Activity in Contact Dermatitis

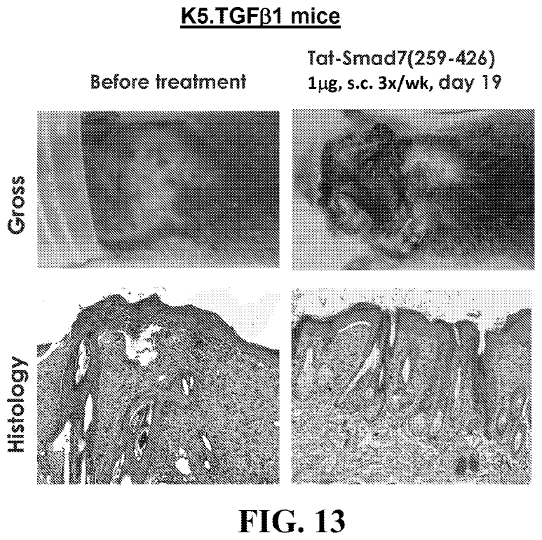

FIG. 12 shows Tat-Smad7(203-217) alleviated Imiquimod-induced inflammation in mouse ear skin, a mouse model for psoriasis and severe contact dermatitis when mast cell infiltrations are present. Each day, 5% Imiquimod cream was applied to mouse ear, and one hour later, the ear was treated topically with or without Tat-Smad7(203-217), 30 μg. An ear biopsy was taken on day 6. Numerous mast cells were present in the Imiquimod-treated ear (untreated control), but there were few mast cells in the Tat-Smad7(203-217)-treated ear. Epidermal hyperplasia was not reversed at this stage, but could be seen after further treatment based on data in presented in FIGS. 10 and 11. Scale bar: 100 μm.

### Example 10: Tat-Smad7(259-426) Shows Activity Treating Skin Inflammation and Fibrosis

FIG. 13 shows that Tat-Smad7(259-426) alleviated TGFβ1-induced skin inflammation and fibrosis. Tat-Smad7(259-426) was injected s.c. 3×/wk, 1 μg in phosphate buffered saline (PBS), into the skin of K5.TGFβ1 mice. Untreated K5.TGFβ1 skin progressively deteriorates. Skin biopsies were taken before (Day 0) and after treatment (Day 19). Inflamed skin gradually improved after treatment and healing was grossly obvious by 3 wks. Similar results were seen by histology, as treated skin showed significant reductions in leukocyte infiltration and fibrosis.

The foregoing discussion of the present technology has been presented for purposes of illustration and description. The foregoing is not intended to limit the present technology to the form or forms disclosed herein. Although the description of the present technology has included description of one or more embodiments and certain variations and modifications, other variations and modifications are within the scope of the present technology, e.g., as may be within the skill and knowledge of those in the art, after understanding the present disclosure. It is intended to obtain rights which include alternative embodiments to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

1. A method of treating or preventing an inflammatory disease state in a subject comprising:
administering to a subject in need of such therapy a fusion protein or a nucleic acid encoding the same, wherein the fusion protein comprises a PTD-Smad7 fusion protein comprising an amino acid sequence selected from the group consisting of SEQ ID NOs: 58, 62, 66, and 70.

2. The method of claim 1, wherein the inflammatory disease state is a wound.

3. The method of claim 2, wherein the wounds are acute.

4. The method of claim 2, wherein the wounds are chronic.

5. The method of claim 2, wherein the wounds include scarring, including keloid formation or hypertrophic scarring.

6. The method of claim 2, wherein the wounds are diabetic wounds.

7. The method of claim 2, wherein the wounds are ulcers.

8. (canceled)

9. (canceled)

10. (canceled)

11. The method of claim 2, wherein the wounds are the result of ischemia.

12. The method of claim 2, wherein the wounds are the result of chemotherapy, radiotherapy, immunotherapy, hormone therapy, toxin therapy, surgery or targeted therapy.

13. The method of claim 2, wherein the wounds are the result of radiation toxicity, including radiation-induced dermatitis, or radiation pneumonitis leading to pulmonary fibrosis.

14. The method of claim 1, wherein the inflammatory disease state is oral mucositis.

15. (canceled)

16. The method of claim 1, wherein the inflammatory disease state is autoimmune diseases or disorders.

17. The method of claim 16, wherein the autoimmune disease or disorder is psoriasis.

18. The method of claim 1, wherein the inflammatory disease state is skin inflammation selected from the group consisting of allergic dermatitis, atopic dermatitis, and contact dermatitis.

19. (canceled)

20. (canceled)

21. (canceled)

22. The method of claim 1, wherein the inflammatory disease state is stomatitis, including recurrent aphthous stomatitis.

23. The method of claim 1, wherein the inflammatory disease state is fibrosis, including idiopathic pulmonary fibrosis, interstitial lung disease, radiation pneumonitis leading to pulmonary fibrosis; and fibrotic lung disease.

24. The method of claim 1, wherein the nucleic acid is an expression vector encoding the fusion protein.

25. (canceled)

26. (canceled)

27. (canceled)

28. The method of claim 1, wherein the PTD is Tat.

29. (canceled)

30. The method of claim 1, wherein the fusion protein is administered topically.

31. (canceled)

32. (canceled)

33. (canceled)

34. A composition comprising a fusion protein, wherein the fusion protein comprises a PTD-Smad7 fusion protein comprising an amino acid sequence selected from the group consisting of SEQ ID NOs: 58, 62, 66, and 70.

35.-100. (canceled)
